SEQUENCE LISTING (628 sequences)

SEQ ID NO: 1 - RNA, 10 nt, Artificial Sequence
  Synthetic
cuuuuaucac

SEQ ID NO: 2 - RNA, 20 nt, Artificial Sequence
  Synthetic
cuaacaguugcuuuuaucac

SEQ ID NO: 3 - RNA, 10 nt, Artificial Sequence
  Synthetic
cacaggcucc

SEQ ID NO: 4 - RNA, 20 nt, Artificial Sequence
  Synthetic
uugcuuuuaucacaggcucc

SEQ ID NO: 5 - RNA, 10 nt, Artificial Sequence
  Synthetic
ggcuccagga

SEQ ID NO: 6 - RNA, 20 nt, Artificial Sequence
  Synthetic
uuuuaucacaggcuccagga

SEQ ID NO: 7 - RNA, 10 nt, Artificial Sequence
  Synthetic
gcuccaggaa

SEQ ID NO: 8 - RNA, 20 nt, Artificial Sequence
  Synthetic
uuuaucacaggcuccaggaa

SEQ ID NO: 9 - RNA, 10 nt, Artificial Sequence
  Synthetic
aggaaggguu

SEQ ID NO: 10 - RNA, 20 nt, Artificial Sequence
  Synthetic
cacaggcuccaggaaggguu

SEQ ID NO: 11 - RNA, 10 nt, Artificial Sequence
  Synthetic
uaacacacca

SEQ ID NO: 12 - RNA, 20 nt, Artificial Sequence
  Synthetic
cucuuagacauaacacacca

SEQ ID NO: 13 - RNA, 10 nt, Artificial Sequence
  Synthetic
auaacacacc

SEQ ID NO: 14 - RNA, 20 nt, Artificial Sequence
  Synthetic
acucuuagacauaacacacc

SEQ ID NO: 15 - RNA, 10 nt, Artificial Sequence
  Synthetic
aauacaacuu

SEQ ID NO: 16 - RNA, 20 nt, Artificial Sequence
  Synthetic
caccagggucaauacaacuu

SEQ ID NO: 17 - RNA, 10 nt, Artificial Sequence
  Synthetic
acaacuuuga

SEQ ID NO: 18 - RNA, 20 nt, Artificial Sequence
  Synthetic
cagggucaauacaacuuuga

SEQ ID NO: 19 - RNA, 10 nt, Artificial Sequence
  Synthetic
cuuugaagcu

SEQ ID NO: 20 - RNA, 20 nt, Artificial Sequence
  Synthetic
gucaauacaacuuugaagcu

SEQ ID NO: 21 - RNA, 10 nt, Artificial Sequence
  Synthetic
aagcuagucu

SEQ ID NO: 22 - RNA, 20 nt, Artificial Sequence
  Synthetic
uacaacuuugaagcuagucu

SEQ ID NO: 23 - RNA, 10 nt, Artificial Sequence
  Synthetic
gcuagucuag

SEQ ID NO: 24 - RNA, 20 nt, Artificial Sequence
  Synthetic
caacuuugaagcuagucuag

SEQ ID NO: 25 - RNA, 10 nt, Artificial Sequence
  Synthetic
gucuagugca

SEQ ID NO: 26 - RNA, 20 nt, Artificial Sequence
  Synthetic
uuugaagcuagucuagugca

SEQ ID NO: 27 - RNA, 10 nt, Artificial Sequence
  Synthetic
gcaagcuaac

SEQ ID NO: 28 - RNA, 20 nt, Artificial Sequence
  Synthetic
cuagucuagugcaagcuaac

SEQ ID NO: 29 - RNA, 10 nt, Artificial Sequence
  Synthetic
agccuugaca

SEQ ID NO: 30 - RNA, 20 nt, Artificial Sequence
  Synthetic
cuugaccaauagccuugaca

SEQ ID NO: 31 - RNA, 10 nt, Artificial Sequence
  Synthetic
acuccaccca

SEQ ID NO: 32 - RNA, 20 nt, Artificial Sequence
  Synthetic
cccuggcuaaacuccaccca

SEQ ID NO: 33 - RNA, 10 nt, Artificial Sequence
  Synthetic
cuccacccau

SEQ ID NO: 34 - RNA, 20 nt, Artificial Sequence
  Synthetic
ccuggcuaaacuccacccau

SEQ ID NO: 35 - RNA, 10 nt, Artificial Sequence
  Synthetic
acccaugggu

SEQ ID NO: 36 - RNA, 20 nt, Artificial Sequence
  Synthetic
gcuaaacuccacccaugggu

SEQ ID NO: 37 - RNA, 10 nt, Artificial Sequence
  Synthetic
gugaucuugu

SEQ ID NO: 38 - RNA, 20 nt, Artificial Sequence
  Synthetic
ggcacacccugugaucuugu

SEQ ID NO: 39 - RNA, 10 nt, Artificial Sequence
  Synthetic
ugugaucuug

SEQ ID NO: 40 - RNA, 20 nt, Artificial Sequence
  Synthetic
gggcacacccugugaucuug

SEQ ID NO: 41 - RNA, 10 nt, Artificial Sequence
  Synthetic
ugagaaggug

SEQ ID NO: 42 - RNA, 20 nt, Artificial Sequence
  Synthetic
gggugugcccugagaaggug

SEQ ID NO: 43 - RNA, 10 nt, Artificial Sequence
  Synthetic
augcacaccc

SEQ ID NO: 44 - RNA, 20 nt, Artificial Sequence
  Synthetic
agcacacaagaugcacaccc

SEQ ID NO: 45 - RNA, 10 nt, Artificial Sequence
  Synthetic
caggcugggc

SEQ ID NO: 46 - RNA, 20 nt, Artificial Sequence
  Synthetic
gaugcacacccaggcugggc

SEQ ID NO: 47 - RNA, 10 nt, Artificial Sequence
  Synthetic
ggcuggacag

SEQ ID NO: 48 - RNA, 20 nt, Artificial Sequence
  Synthetic
acccaggcugggcuggacag

SEQ ID NO: 49 - RNA, 10 nt, Artificial Sequence
  Synthetic
ugugcuuggu

SEQ ID NO: 50 - RNA, 20 nt, Artificial Sequence
  Synthetic
gugcaucuugugugcuuggu

SEQ ID NO: 51 - RNA, 10 nt, Artificial Sequence
  Synthetic
cccacccugc

SEQ ID NO: 52 - RNA, 20 nt, Artificial Sequence
  Synthetic
ucaccaggaccccacccugc

SEQ ID NO: 53 - RNA, 10 nt, Artificial Sequence
  Synthetic
gcugaaaccc

SEQ ID NO: 54 - RNA, 20 nt, Artificial Sequence
  Synthetic
accccacccugcugaaaccc

SEQ ID NO: 55 - RNA, 10 nt, Artificial Sequence
  Synthetic
caccuucuca

SEQ ID NO: 56 - RNA, 20 nt, Artificial Sequence
  Synthetic
gugagcuccccaccuucuca

SEQ ID NO: 57 - RNA, 10 nt, Artificial Sequence
  Synthetic
caaugcuugg

SEQ ID NO: 58 - RNA, 20 nt, Artificial Sequence
  Synthetic
caggaugaugcaaugcuugg

SEQ ID NO: 59 - RNA, 10 nt, Artificial Sequence
  Synthetic
augcaaugcu

SEQ ID NO: 60 - RNA, 20 nt, Artificial Sequence
  Synthetic
uaccaggaugaugcaaugcu

SEQ ID NO: 61 - RNA, 10 nt, Artificial Sequence
  Synthetic
uccugguacc

SEQ ID NO: 62 - RNA, 20 nt, Artificial Sequence
  Synthetic
cccauugccuuccugguacc

SEQ ID NO: 63 - RNA, 10 nt, Artificial Sequence
  Synthetic
caccuucuca

SEQ ID NO: 64 - RNA, 20 nt, Artificial Sequence
  Synthetic
gugagcuccccaccuucuca

SEQ ID NO: 65 - RNA, 10 nt, Artificial Sequence
  Synthetic
caaugcuugg

SEQ ID NO: 66 - RNA, 20 nt, Artificial Sequence
  Synthetic
caggaugaugcaaugcuugg

SEQ ID NO: 67 - RNA, 10 nt, Artificial Sequence
  Synthetic
augcaaugcu

SEQ ID NO: 68 - RNA, 20 nt, Artificial Sequence
  Synthetic
uaccaggaugaugcaaugcu

SEQ ID NO: 69 - RNA, 10 nt, Artificial Sequence
  Synthetic
uccugguacc

SEQ ID NO: 70 - RNA, 20 nt, Artificial Sequence
  Synthetic
cccauugccuuccugguacc

SEQ ID NO: 71 - RNA, 10 nt, Artificial Sequence
  Synthetic
ggcuggacag

SEQ ID NO: 72 - RNA, 20 nt, Artificial Sequence
  Synthetic
acccaggcugggcuggacag

SEQ ID NO: 73 - RNA, 10 nt, Artificial Sequence
  Synthetic
caggcugggc

SEQ ID NO: 74 - RNA, 20 nt, Artificial Sequence
  Synthetic
gaugcacacccaggcugggc

SEQ ID NO: 75 - RNA, 10 nt, Artificial Sequence
  Synthetic
cacccaggcu

SEQ ID NO: 76 - RNA, 20 nt, Artificial Sequence
  Synthetic
acaagaugcacacccaggcu

SEQ ID NO: 77 - RNA, 10 nt, Artificial Sequence
  Synthetic
acacccaggc

SEQ ID NO: 78 - RNA, 20 nt, Artificial Sequence
  Synthetic
cacaagaugcacacccaggc

SEQ ID NO: 79 - RNA, 10 nt, Artificial Sequence
  Synthetic
augcacaccc

SEQ ID NO: 80 - RNA, 20 nt, Artificial Sequence
  Synthetic
agcacacaagaugcacaccc

SEQ ID NO: 81 - RNA, 10 nt, Artificial Sequence
  Synthetic
gaccgcucac

SEQ ID NO: 82 - RNA, 20 nt, Artificial Sequence
  Synthetic
cagccuuggggaccgcucac

SEQ ID NO: 83 - RNA, 10 nt, Artificial Sequence
  Synthetic
cacagccuug

SEQ ID NO: 84 - RNA, 20 nt, Artificial Sequence
  Synthetic
gaaggcugggcacagccuug

SEQ ID NO: 85 - RNA, 10 nt, Artificial Sequence
  Synthetic
cucccuaccg

SEQ ID NO: 86 - RNA, 20 nt, Artificial Sequence
  Synthetic
gugccgacaacucccuaccg

SEQ ID NO: 87 - RNA, 10 nt, Artificial Sequence
  Synthetic
gugaucuugu

SEQ ID NO: 88 - RNA, 20 nt, Artificial Sequence
  Synthetic
ggcacacccugugaucuugu

SEQ ID NO: 89 - RNA, 10 nt, Artificial Sequence
  Synthetic
ugugaucuug

SEQ ID NO: 90 - RNA, 20 nt, Artificial Sequence
  Synthetic
gggcacacccugugaucuug

SEQ ID NO: 91 - RNA, 10 nt, Artificial Sequence
  Synthetic
caccuucuca

SEQ ID NO: 92 - RNA, 20 nt, Artificial Sequence
  Synthetic
gugagcuccccaccuucuca

SEQ ID NO: 93 - RNA, 10 nt, Artificial Sequence
  Synthetic
caaugcuugg

SEQ ID NO: 94 - RNA, 20 nt, Artificial Sequence
  Synthetic
caggaugaugcaaugcuugg

SEQ ID NO: 95 - RNA, 10 nt, Artificial Sequence
  Synthetic
augcaaugcu

SEQ ID NO: 96 - RNA, 20 nt, Artificial Sequence
  Synthetic
uaccaggaugaugcaaugcu

SEQ ID NO: 97 - RNA, 10 nt, Artificial Sequence
  Synthetic
uccugguacc

SEQ ID NO: 98 - RNA, 20 nt, Artificial Sequence
  Synthetic
cccauugccuuccugguacc

SEQ ID NO: 99 - RNA, 10 nt, Artificial Sequence
  Synthetic
gaccccuauc

SEQ ID NO: 100 - RNA, 20 nt, Artificial Sequence
  Synthetic
ucccuaccgcgaccccuauc

SEQ ID NO: 101 - RNA, 10 nt, Artificial Sequence
  Synthetic
ccccuaucag

SEQ ID NO: 102 - RNA, 20 nt, Artificial Sequence
  Synthetic
ccuaccgcgaccccuaucag

SEQ ID NO: 103 - RNA, 10 nt, Artificial Sequence
  Synthetic
cuaucagugc

SEQ ID NO: 104 - RNA, 20 nt, Artificial Sequence
  Synthetic
accgcgaccccuaucagugc

SEQ ID NO: 105 - RNA, 10 nt, Artificial Sequence
  Synthetic
cagccaggac

SEQ ID NO: 106 - RNA, 20 nt, Artificial Sequence
  Synthetic
ggccaucugccagccaggac

SEQ ID NO: 107 - RNA, 10 nt, Artificial Sequence
  Synthetic
ucugccagcc

SEQ ID NO: 108 - RNA, 20 nt, Artificial Sequence
  Synthetic
aagguggccaucugccagcc

SEQ ID NO: 109 - RNA, 10 nt, Artificial Sequence
  Synthetic
agcaagaagg

SEQ ID NO: 110 - RNA, 20 nt, Artificial Sequence
  Synthetic
gugaaaacgcagcaagaagg

SEQ ID NO: 111 - RNA, 10 nt, Artificial Sequence
  Synthetic
cgcagcaaga

SEQ ID NO: 112 - RNA, 20 nt, Artificial Sequence
  Synthetic
caugugaaaacgcagcaaga

SEQ ID NO: 113 - RNA, 10 nt, Artificial Sequence
  Synthetic
aaacgcagca

SEQ ID NO: 114 - RNA, 20 nt, Artificial Sequence
  Synthetic
ugccaugugaaaacgcagca

SEQ ID NO: 115 - RNA, 10 nt, Artificial Sequence
  Synthetic
gugaaaacgc

SEQ ID NO: 116 - RNA, 20 nt, Artificial Sequence
  Synthetic
ucucugccaugugaaaacgc

SEQ ID NO: 117 - RNA, 10 nt, Artificial Sequence
  Synthetic
gguggcacac

SEQ ID NO: 118 - RNA, 20 nt, Artificial Sequence
  Synthetic
ccagcuaucagguggcacac

SEQ ID NO: 119 - RNA, 10 nt, Artificial Sequence
  Synthetic
ggcacacagc

SEQ ID NO: 120 - RNA, 20 nt, Artificial Sequence
  Synthetic
gcuaucagguggcacacagc

SEQ ID NO: 121 - RNA, 10 nt, Artificial Sequence
  Synthetic
acacagcugc

SEQ ID NO: 122 - RNA, 20 nt, Artificial Sequence
  Synthetic
aucagguggcacacagcugc

SEQ ID NO: 123 - RNA, 10 nt, Artificial Sequence
  Synthetic
cacagcuggu

SEQ ID NO: 124 - RNA, 20 nt, Artificial Sequence
  Synthetic
gacagcugugcacagcuggu

SEQ ID NO: 125 - RNA, 10 nt, Artificial Sequence
  Synthetic
ugugcacagc

SEQ ID NO: 126 - RNA, 20 nt, Artificial Sequence
  Synthetic
uggggacagcugugcacagc

SEQ ID NO: 127 - RNA, 10 nt, Artificial Sequence
  Synthetic
ugcggccaug

SEQ ID NO: 128 - RNA, 20 nt, Artificial Sequence
  Synthetic
ggaggcacugugcggccaug

SEQ ID NO: 129 - RNA, 10 nt, Artificial Sequence
  Synthetic
ugugcggcca

SEQ ID NO: 130 - RNA, 20 nt, Artificial Sequence
  Synthetic
ggggaggcacugugcggcca

SEQ ID NO: 131 - RNA, 10 nt, Artificial Sequence
  Synthetic
ggaacagcug

SEQ ID NO: 132 - RNA, 20 nt, Artificial Sequence
  Synthetic
ugcaggacagggaacagcug

SEQ ID NO: 133 - RNA, 10 nt, Artificial Sequence
  Synthetic
agggaacagc

SEQ ID NO: 134 - RNA, 20 nt, Artificial Sequence
  Synthetic
gcugcaggacagggaacagc

SEQ ID NO: 135 - RNA, 10 nt, Artificial Sequence
  Synthetic
gcugcaggac

SEQ ID NO: 136 - RNA, 20 nt, Artificial Sequence
  Synthetic
aagcagcccagcugcaggac

SEQ ID NO: 137 - RNA, 10 nt, Artificial Sequence
  Synthetic
gcccagcugc

SEQ ID NO: 138 - RNA, 20 nt, Artificial Sequence
  Synthetic
caauuaagcagcccagcugc

SEQ ID NO: 139 - RNA, 10 nt, Artificial Sequence
  Synthetic
ccucccggug

SEQ ID NO: 140 - RNA, 20 nt, Artificial Sequence
  Synthetic
auuucucuuuccucccggug

SEQ ID NO: 141 - RNA, 10 nt, Artificial Sequence
  Synthetic
gugaggacac

SEQ ID NO: 142 - RNA, 20 nt, Artificial Sequence
  Synthetic
uuuccucccggugaggacac

SEQ ID NO: 143 - RNA, 10 nt, Artificial Sequence
  Synthetic
gaggacacag

SEQ ID NO: 144 - RNA, 20 nt, Artificial Sequence
  Synthetic
uccucccggugaggacacag

SEQ ID NO: 145 - RNA, 10 nt, Artificial Sequence
  Synthetic
ggacacagug

SEQ ID NO: 146 - RNA, 20 nt, Artificial Sequence
  Synthetic
cucccggugaggacacagug

SEQ ID NO: 147 - RNA, 10 nt, Artificial Sequence
  Synthetic
gaaagagaaa

SEQ ID NO: 148 - RNA, 20 nt, Artificial Sequence
  Synthetic
ucaccgggaggaaagagaaa

SEQ ID NO: 149 - RNA, 10 nt, Artificial Sequence
  Synthetic
acuaucucaa

SEQ ID NO: 150 - RNA, 20 nt, Artificial Sequence
  Synthetic
ccuuccccacacuaucucaa

SEQ ID NO: 151 - RNA, 10 nt, Artificial Sequence
  Synthetic
agaagcagag

SEQ ID NO: 152 - RNA, 20 nt, Artificial Sequence
  Synthetic
uuuccuuaucagaagcagag

SEQ ID NO: 153 - RNA, 10 nt, Artificial Sequence
  Synthetic
uuuccuuauc

SEQ ID NO: 154 - RNA, 20 nt, Artificial Sequence
  Synthetic
auaaaauuauuuuccuuauc

SEQ ID NO: 155 - RNA, 10 nt, Artificial Sequence
  Synthetic
ucauaagagc

SEQ ID NO: 156 - RNA, 20 nt, Artificial Sequence
  Synthetic
uuagaugagcucauaagagc

SEQ ID NO: 157 - RNA, 10 nt, Artificial Sequence
  Synthetic
aaaaguaauu

SEQ ID NO: 158 - RNA, 20 nt, Artificial Sequence
  Synthetic
gaggcuuuugaaaaguaauu

SEQ ID NO: 159 - RNA, 10 nt, Artificial Sequence
  Synthetic
ucuguggggg

SEQ ID NO: 160 - RNA, 20 nt, Artificial Sequence
  Synthetic
acugaccuuaucuguggggg

SEQ ID NO: 161 - RNA, 10 nt, Artificial Sequence
  Synthetic
uuaucugugg

SEQ ID NO: 162 - RNA, 20 nt, Artificial Sequence
  Synthetic
ccaacugaccuuaucugugg

SEQ ID NO: 163 - RNA, 10 nt, Artificial Sequence
  Synthetic
accuuaucug

SEQ ID NO: 164 - RNA, 20 nt, Artificial Sequence
  Synthetic
cucccaacugaccuuaucug

SEQ ID NO: 165 - RNA, 10 nt, Artificial Sequence
  Synthetic
uugcagaugg

SEQ ID NO: 166 - RNA, 20 nt, Artificial Sequence
  Synthetic
ucuuccuggauugcagaugg

SEQ ID NO: 167 - RNA, 10 nt, Artificial Sequence
  Synthetic
ggauugcaga

SEQ ID NO: 168 - RNA, 20 nt, Artificial Sequence
  Synthetic
uucucuuccuggauugcaga

SEQ ID NO: 169 - RNA, 10 nt, Artificial Sequence
  Synthetic
guucucuucc

SEQ ID NO: 170 - RNA, 20 nt, Artificial Sequence
  Synthetic
cguggucaggguucucuucc

SEQ ID NO: 171 - RNA, 10 nt, Artificial Sequence
  Synthetic
cucgugguca

SEQ ID NO: 172 - RNA, 20 nt, Artificial Sequence
  Synthetic
ugaaggcugacucgugguca

SEQ ID NO: 173 - RNA, 10 nt, Artificial Sequence
  Synthetic
acucgugguc

SEQ ID NO: 174 - RNA, 20 nt, Artificial Sequence
  Synthetic
cugaaggcugacucgugguc

SEQ ID NO: 175 - RNA, 10 nt, Artificial Sequence
  Synthetic
ggcugacucg

SEQ ID NO: 176 - RNA, 20 nt, Artificial Sequence
  Synthetic
cauuucugaaggcugacucg

SEQ ID NO: 177 - RNA, 10 nt, Artificial Sequence
  Synthetic
acauuucuga

SEQ ID NO: 178 - RNA, 20 nt, Artificial Sequence
  Synthetic
guuuuuucucacauuucuga

SEQ ID NO: 179 - RNA, 10 nt, Artificial Sequence
  Synthetic
auccucuugg

SEQ ID NO: 180 - RNA, 20 nt, Artificial Sequence
  Synthetic
aauuagcaguauccucuugg

SEQ ID NO: 181 - RNA, 10 nt, Artificial Sequence
  Synthetic
aguauccucu

SEQ ID NO: 182 - RNA, 20 nt, Artificial Sequence
  Synthetic
aaaaauuagcaguauccucu

SEQ ID NO: 183 - RNA, 10 nt, Artificial Sequence
  Synthetic
aaaaaaaauu

SEQ ID NO: 184 - RNA, 20 nt, Artificial Sequence
  Synthetic
caaaggcuauaaaaaaaauu

SEQ ID NO: 185 - RNA, 10 nt, Artificial Sequence
  Synthetic
aacaaggcaa

SEQ ID NO: 186 - RNA, 20 nt, Artificial Sequence
  Synthetic
acugaaucggaacaaggcaa

SEQ ID NO: 187 - RNA, 10 nt, Artificial Sequence
  Synthetic
aaucggaaca

SEQ ID NO: 188 - RNA, 20 nt, Artificial Sequence
  Synthetic
ggaaugacugaaucggaaca

SEQ ID NO: 189 - RNA, 10 nt, Artificial Sequence
  Synthetic
augacugaau

SEQ ID NO: 190 - RNA, 20 nt, Artificial Sequence
  Synthetic
aaaaacuggaaugacugaau

SEQ ID NO: 191 - protein, 223 aa, Artificial Sequence
  Synthetic
MSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 192 - protein, 164 aa, Artificial Sequence
  Synthetic
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEG

SEQ ID NO: 193 - protein, 166 aa, Artificial Sequence
  Synthetic
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQ

SEQ ID NO: 194 - protein, 119 aa, Artificial Sequence
  Synthetic
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 195 - protein, 116 aa, Artificial Sequence
  Synthetic
MKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQ

SEQ ID NO: 196 - protein, 7 aa, Artificial Sequence
  Synthetic
ENLYFQS

SEQ ID NO: 197 - protein, 246 aa, Artificial Sequence
  Synthetic
MASSNSMFRGLRDYNPISNNICHLTNVSDGASNSLYGVGFGPLILTNRHLFERNNGELVIKSRHGEFVIKNTTQLHLLPIPDRDLLLIRLPKDVPPFPQKLGFRQPEKGERICMVGSNFQTKSITSIVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPSGLFKVSKLISDLDSTAVYAQ

SEQ ID NO: 198 - protein, 7 aa, Artificial Sequence
  Synthetic
GGCSHQS

SEQ ID NO: 199 - protein, 246 aa, Artificial Sequence
  Synthetic
MASSKSLFRGLRDYNPIASSICQLNNSSGARQSEMFGLGFGGLIVTNQHLFKRNDGELTIRSHHGEFVVKDTKTLKLLPCKGRDIVIIRLPKDFPPFPRRLQFRTPTTEDRVCLIGSNFQTKSISSTMSETSATYPVDNSHFWKHWISTKDGHCGLPIVSTRDGSILGLHSLANSTNTQNFYAAFPDNFETTYLSNQDNDNWIKQWRYNPDEVCWGSLQLKRDIPQSPFTICKLLTDLDGEFVYTQ

SEQ ID NO: 200 - protein, 8 aa, Artificial Sequence
  Synthetic
QVVVHQSK

SEQ ID NO: 201 - protein, 247 aa, Artificial Sequence
  Synthetic
MASAKSLMRGLRDFNPIAQTVCRLKVSVEYGASEMYGFGFGAYIVANHHLFRSYNGSMEVQSMHGTFRVKNLHSLSVLPIKGRDIILIKMPKDFPVFPQKLHFRAPTQNERICLVGTNFQEKYASSIITETSTTYNIPGSTFWKHWIETDNGHCGLPVVSTADGCIVGIHSLANNAHTTNYYSAFDEDFESKYLRTNEHNEWVKSWVYNPDTVLWGPLKLKDSTPKGLFKTTKLVQDLIDHDVVVEQ

SEQ ID NO: 202 - protein, 8 aa, Artificial Sequence
  Synthetic
YDVRHQSR

SEQ ID NO: 203 - protein, 239 aa, Artificial Sequence
  Synthetic
MASDMYIERAGDITWEKDAEVTGNSPRLDVALDESGDFSLVEEDGPPMREGGGGSGGGGSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFE

SEQ ID NO: 204 - protein, 8 aa, Artificial Sequence
  Synthetic
KERKRRGA

SEQ ID NO: 205 - protein, 243 aa, Artificial Sequence
  Synthetic
MASSTDMWIERTADISWESDAEITGSSERVDVRLDDDGNFQLMNDPGAPWKGGGGSGGGGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEML

SEQ ID NO: 206 - protein, 8 aa, Artificial Sequence
  Synthetic
KQKKRGGK

SEQ ID NO: 207 - RNA, 19 nt, Artificial Sequence
  Synthetic
acaugaggaucacccaugu

SEQ ID NO: 208 - RNA, 144 nt, Artificial Sequence
  Synthetic
guuugagagcuaggccauuacaugaggaucacccauguuucugcagggccuagcaaguucaaauaaggcuaguccguuaucaacuuguugccaacaugaggaucacccaugucugcagggccuuaaguggcaccgagucggugc

SEQ ID NO: 209 - RNA, 25 nt, Artificial Sequence
  Synthetic
ggagcagacgauauggcgucgcucc

SEQ ID NO: 210 - RNA, 122 nt, Artificial Sequence
  Synthetic
guuugagagcuaccggagcagacgauauggcgucgcuccgguagcaaguucaaauaaggcuaguccguuaucaacuuggagcagacgauauggcgucgcuccaaguggcaccgagucggugc

SEQ ID NO: 211 - RNA, 15 nt, Artificial Sequence
  Synthetic
gcccugaagaagggc

SEQ ID NO: 212 - RNA, 106 nt, Artificial Sequence
  Synthetic
guuugagagcuagggcccugaagaagggcccuagcaaguucaaauaaggcuaguccguuaucaacuugggcccugaagaagggcccaaguggcaccgagucggugc

SEQ ID NO: 213 - protein, 117 aa, Artificial Sequence
  Synthetic
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 214 - protein, 123 aa, Artificial Sequence
  Synthetic
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 215 - protein, 23 aa, Artificial Sequence
  Synthetic
MGNARTRRRERRAEKQAQWKAAN

SEQ ID NO: 216 - protein, 83 aa, Artificial Sequence
  Synthetic
TNLSDIIEKETGKQLVIQESILMLPEEVEEVIGNKPESDILVHTAYDESTDENVMLLTSDAPEYKPWALVIQDSNGENKIKML

SEQ ID NO: 217 - protein, 22 aa, Artificial Sequence
  Synthetic
GSGATNFSLLKQAGDVEENPGP

SEQ ID NO: 218 - protein, 21 aa, Artificial Sequence
  Synthetic
GSGEGRGSLLTCGDVEENPGP

SEQ ID NO: 219 - protein, 23 aa, Artificial Sequence
  Synthetic
GSGQCTNYALLKLAGDVESNPGP

SEQ ID NO: 220 - protein, 74 aa, Artificial Sequence
  Synthetic
SEKGKQHAEILFLDKIRSMELSQVTITCYLTWSPCPNCAWQLAAFKRDRPDLILHIYTSRLYFHWKRPFQKGLC

SEQ ID NO: 221 - protein, 74 aa, Artificial Sequence
  Synthetic
SEKGKQHAEILFLDKIRSMELSQVTITCYLTWSPCPNCAWQLAAFKRDRPDLIPHIYTSRLYFHWKRPFQKGLC

SEQ ID NO: 222 - protein, 74 aa, Artificial Sequence
  Synthetic
SEKGKQHAEILFLDKIRSMELSQVTITCYLTWSPCPNCAWRLAAFKRDRPDLILHIYTSRLYFHWKRPFQKGLC

SEQ ID NO: 223 - protein, 74 aa, Artificial Sequence
  Synthetic
SEKGKQHAEILFLNKIRSMELSQVTITCYLTWSPCPNCAWQLAAFKKDRPDLILHIYTSRLYFHWKRPFQKGLC

SEQ ID NO: 224 - protein, 74 aa, Artificial Sequence
  Synthetic
SKKGKQHAEILFLDKIRSMELSQVTITCYLTWSPCPNCAWQLAAFKRDHPDLILHIYTSRLYFHWKRPFQKGLC

SEQ ID NO: 225 - protein, 74 aa, Artificial Sequence
  Synthetic
SKKGKQHAEILFLEKIRSMELSQMRITCYLTWSPCPNCAWQLAAFQKDRPDLILHIYTSRLYFHWRRIFQKGLC

SEQ ID NO: 226 - protein, 74 aa, Artificial Sequence; identical to SEQ ID NO: 225
  Synthetic

SEQ ID NO: 227 - protein, 74 aa, Artificial Sequence
  Synthetic
SKKGKQHAEILFLDKIRSMELSQVRITCYLTWSPCPNCAWQLETFKKDRPDLILHIYTSRLYFHWKRAFQEGLC

SEQ ID NO: 228 - protein, 75 aa, Artificial Sequence
  Synthetic
SKKGKPHAEILFLDKMWSMEELSQVRITCYLTWSPCPNCARQLAAFKKDHPGLILRIYTSRLYFYWRRKFQKGLC

SEQ ID NO: 229 - protein, 73 aa, Artificial Sequence
  Synthetic
KKGEQHVEILFLEKMRSMELSQVRITCYLTWSPCPNCARQLAAFKKDHPDLILRIYTSRLYFYWRKKFQKGLC

SEQ ID NO: 230 - protein, 74 aa, Artificial Sequence
  Synthetic
SKKGRQHAEILFLEKVRSMQLSQVRITCYLTWSPCPNCAWQLAAFKMDHPDLILRIYASRLYFHWRRAFQKGLC

SEQ ID NO: 231 - protein, 73 aa, Artificial Sequence
  Synthetic
NKKGKHAEILFIDEMRSLELGQVQITCYLTWSPCPNCAQELAAFKSDHPDLVLRIYTSRLYFHWRRKYQEGLC

SEQ ID NO: 232 - protein, 73 aa, Artificial Sequence
  Synthetic
NKKGKHAEILFIDEMRSLELGQARITCYLTWSPCPNCAQKLAAFKKDHPDLVLRVYTSRLYFHWRRKYQEGLC

SEQ ID NO: 233 - protein, 73 aa, Artificial Sequence
  Synthetic
NKKDKHAEILFIDKMRSLELCQVRITCYLTWSPCPNCAQELAAFKKDHPDLVLRIYTSRLYFHWRRKYQEGLC

SEQ ID NO: 234 - protein, 73 aa, Artificial Sequence
  Synthetic
NKKGKHAEILFIDEMRSLKLSQERITCYLTWSPCPNCAQELAAFKRDHPGLVLRIYASRLYFHWRRKYQEGLC

SEQ ID NO: 235 - protein, 72 aa, Artificial Sequence
  Synthetic
NKRAKHAEILLIDMMRSMELGQVQITCYITWSPCPTCAQELAAFKQDHPDLVLRIYASRLYFHWKRKFQKGL

SEQ ID NO: 236 - protein, 73 aa, Artificial Sequence
  Synthetic
NKKGRHAEICLIDEMRSLGLGKAQITCYLTWSPCRKCAQELATFKKDHPDLVLRVYASRLYFHWSRKYQQGLC

SEQ ID NO: 237 - protein, 75 aa, Artificial Sequence
  Synthetic
NKKGHHAEIRFIERIRSMGLDPSQDYQITCYLTWSPCLDCAFKLAKLKKDFPRLTLRIFTSRLYFHWIRKFQKGL

SEQ ID NO: 238 - protein, 72 aa, Artificial Sequence
  Synthetic
NKKGKHAEARFVDKMRSMQLDHALITCYLTWSPCLDCSQKLAALKRDHPGLTLRIFTSRLYFHWVKKFQEGL

SEQ ID NO: 239 - protein, 76 aa, Artificial Sequence
  Synthetic
SPQKGHHAESRFIKRISSMDLDRSRSYQITCFLTWSPCPSCAQELASFKRAHPHLRFQIFVSRLYFHWKRSYQAGL

SEQ ID NO: 240 - protein, 74 aa, Artificial Sequence
  Synthetic
KKGYHAESRFIKRICSMDLGQDQSYQVTCFLTWSPCPHCAQELVSFKRAHPHLRLQIFTARLFFHWKRSYQEGL

SEQ ID NO: 241 - protein, 74 aa, Artificial Sequence
  Synthetic
KKGQHAEIRFIERIHSMALDQARSYQITCFLTWSPCPFCAQELASFKSTHPRVHLQIFVSRLYFHWKRSYQEGL

SEQ ID NO: 242 - protein, 75 aa, Artificial Sequence
  Synthetic
NKKGHHAEIRFIKKIRSLDLDQSQNYEVTCYLTWSPCPDCAQELVALTRSHPHVRLRLFTSRLYFHWFWSFQEGL

SEQ ID NO: 243 - protein, 72 aa, Artificial Sequence
  Synthetic
NRHAEICFIDEIESMGLDKTQCYEVTCYLTWSPCPSCAQKLAAFTKAQVHLNLRIFASRLYYHWRSSYQKGL

SEQ ID NO: 244 - protein, 72 aa, Artificial Sequence
  Synthetic
NRHAEICFIDEIESMGLDKTQCYEVTCYLTWSPCPSCAQKLVAFAKAQDHLNLRIFASRLYYHWRRRYKEGL

SEQ ID NO: 245 - protein, 70 aa, Artificial Sequence
  Synthetic
HVEICFIDKIASMELDKTQCYDVTCYLTWSPCPSCAQKLAAFAKAQDHLNLRIFASRLYYHWRRSYQKGL

SEQ ID NO: 246 - protein, 75 aa, Artificial Sequence
  Synthetic
NKKKCHAEICFINEIKSMGLDETQCYQVTCYLTWSPCSSCAWELVDFIKAHDHLNLGIFASRLYYHWCKPQQKGL

SEQ ID NO: 247 - protein, 76 aa, Artificial Sequence
  Synthetic
ENKKKCHAEICFINEIKSMGLDETQCYQVTCYLTWSPCSSCAWELVDFIKAHDHLNLGIFASRLYYHWCKPQQKGL

SEQ ID NO: 248 - protein, 75 aa, Artificial Sequence
  Synthetic
NKKKCHAEICFINEIKSMGLDETQCYQVTCYLTWSPCSSCAWKLVDFIQAHDHLNLRIFASRLYYHWCKPQQEGL

SEQ ID NO: 249 - protein, 75 aa, Artificial Sequence
  Synthetic
NKKKRHAEIRFINKIKSMGLDETQCYQVTCYLTWSPCPSCAWELVDFIKAHDHLNLGIFASRLYYHWCRHQQEGL

SEQ ID NO: 250 - protein, 75 aa, Artificial Sequence
  Synthetic
NKKKDHAEIRFINKIKSMGLDETQCYQVTCYLTWSPCPSCAGELVDFIKAHRHLNLRIFASRLYYHWRPNYQEGL

SEQ ID NO: 251 - protein, 75 aa, Artificial Sequence
  Synthetic
NKKKEHAEIRFINKIKSMGLDETQCYQVTCYLTWSPCPSCAGKLVDFIKAHHHLNLRIFASRLYYHWRPNYQEGL

SEQ ID NO: 252 - protein, 75 aa, Artificial Sequence
  Synthetic
NKKKHHAEIHFINKIKSMGLDETQCYQVTCYLTWSPCPSCARELVDFIKAHRHLNLRIFASRLYYHWRPHYQEGL

SEQ ID NO: 253 - protein, 75 aa, Artificial Sequence
  Synthetic
NKKQRHAEIRFIDKINSLDLNPSQSYKIICYITWSPCPNCANELVNFITRNNHLKLEIFASRLYFHWIKPFKMGL

SEQ ID NO: 254 - protein, 75 aa, Artificial Sequence
  Synthetic
NKKQRHAEIRFIDKINSLDLNPSQSYKIICYITWSPCPNCASELVDFITRNDHLDLQIFASRLYFHWIKPFKRGL

SEQ ID NO: 255 - protein, 75 aa, Artificial Sequence
  Synthetic
NKKQRHAEIRFIDKINSLNLDRRQSYKIICYITWSPCPRCASELVDFITGNDHLNLQIFASRLYFHWKKPFQRGL

SEQ ID NO: 256 - protein, 75 aa, Artificial Sequence
  Synthetic
NKKKRHAEIRFIDKINSLNLDQNQCYRIICYVTWSPCHNCAKELVDFISNRHHLSLQLFASRLYFHWVRCYQRGL

SEQ ID NO: 257 - protein, 75 aa, Artificial Sequence
  Synthetic
NKKKRHAEIRFIDKIKSLGLDRVQSYEITCYITWSPCPTCALELVAFTRDYPRLSLQIFASRLYFHWRRRSIQGL

SEQ ID NO: 258 - protein, 75 aa, Artificial Sequence
  Synthetic
NKKKRHAEIRFIDKINSLGLDQDQSYEITCYVTWSPCATCACKLIKFTRKFPNLSLRIFVSRLYYHWFRQNQQGL

SEQ ID NO: 259 - protein, 74 aa, Artificial Sequence
  Synthetic
KKKRHAEIRFIDSIRALQLDQSQRFEITCYLTWSPCPTCAKELAMFVQDHPHISLRLFASRLYFHWRWKYQEGL

SEQ ID NO: 260 - protein, 74 aa, Artificial Sequence
  Synthetic
KKKRHAEIRFIDNIKALRLDTSQRFEITCYVTWSPCPTCAKELVAFVRDHRHISLRLFASRLYFHWLRENKKGL

SEQ ID NO: 261 - protein, 75 aa, Artificial Sequence
  Synthetic
NKKKRHAEIRFIDKIRSLQRDSSQTFEITCYVTWSPCFTCAEELVAFVRDHPHVRLRLFASRLYFHWLRKYQEGL

SEQ ID NO: 262 - protein, 75 aa, Artificial Sequence
  Synthetic
NKKKRHAEICFIDKIKSLTRDTSQRFEIICYITWSPCPFCAEELVAFVKDNPHLSLRIFASRLYVHWRWKYQQGL

SEQ ID NO: 263 - protein, 75 aa, Artificial Sequence; identical to SEQ ID NO: 262
  Synthetic

SEQ ID NO: 264 - protein, 75 aa, Artificial Sequence
  Synthetic
NKKHRHAEVRFIAKIRSMSLDLDQKHQLTCYLTWSPCPSCAQELVTFMAESRHLNLQVFVSRLYFHWQRDFQQGL

SEQ ID NO: 265 - protein, 108 aa, Artificial Sequence
  Synthetic
RRNTVWLCYEVKTKGPSRPRLDTKIFRGQVYFEPQYHAEMCFLSWFCGNQLPAYKCFQITWFVSWTPCPDCVAKLAEFLAEHPNVTLTISAARLYYYWERDYRRALCR

SEQ ID NO: 266 - protein, 108 aa, Artificial Sequence
  Synthetic
RRNTVWLCYEVKTKGPSRPRLDTKIFRGQVYFQFENHAEMCFLSWFCGNQLPAYKCFQITWFVSWTPCPDCVAKLAEFLAEHPNVTLTISAARLYYYWERDYRRALCR

SEQ ID NO: 267 - protein, 108 aa, Artificial Sequence
  Synthetic
RRNTVWLCYEVKTRGPSMPTWGAKIFRGQVYFEPQYHAEMCFLSWFCGNQLPAYKCFQITWFVSWTPCPDCVGKVAEFLAEHPNVTLTISAARLYYYWETDYRRALCR

SEQ ID NO: 268 - protein, 108 aa, Artificial Sequence
  Synthetic
RRNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCR

SEQ ID NO: 269 - protein, 108 aa, Artificial Sequence
  Synthetic
RRNTVWLCYEVKTRGPSVPTWGTKIFRGQVYSKPEHHAEMCFLSWFCGNQLPTYKRFQITWFVSWTPCPDCVAKVAEFLAEHPNVTLTISAARLYYYWETDYRRALCR

SEQ ID NO: 270 - protein, 108 aa, Artificial Sequence
  Synthetic
RRNTVWLCYEVKTRGPSMPTWGAKIFRGQVYFEPQYHAEMCFLSWFCGNQLPAYKRFQITWFVSWTPCPDCVAKVAEFLAEHPNVTLTISAARLYYYWETDYRRALCR

SEQ ID NO: 271 - protein, 108 aa, Artificial Sequence; identical to SEQ ID NO: 270
  Synthetic

SEQ ID NO: 272 - protein, 108 aa, Artificial Sequence; identical to SEQ ID NO: 270
  Synthetic

SEQ ID NO: 273 - protein, 108 aa, Artificial Sequence
  Synthetic
RRNTVWLCYEVKTRGPSMPTWDTKIFRGQVYSKPEHHAEMCFLSRFCGNQLPAYKRFQITWFVSWTPCTDCVAKVAEFLAEHPNVTLTISAARLYYYWETDYRRALCR

SEQ ID NO: 274 - protein, 108 aa, Artificial Sequence
  Synthetic
RRNTVWLCYEVKTRGPSMPTWGAKIFRGQVYFEPQYHAEMCFLSWFCGNQLPAYKRFRITWFVSWTPCPDCVAKVAEFLAEHPNVTLTISAARLYYYWETDYRRALCR

SEQ ID NO: 275 - protein, 99 aa, Artificial Sequence
  Synthetic
RRNTVWLCYEVKTKGPSRPPLDAKIFRGQVYFEPQYHAEMCFLSWFCGNQLPAYKCFQITWFVSWTPCPDCVAKLAEFLAEHPNVTLTISAARLYYYWE

SEQ ID NO: 276 - protein, 108 aa, Artificial Sequence
  Synthetic
RRNTVWLCYEVKTRGPSMPTWDAKIFRGQVYFQPQYHAEMCFLSRFCGNQLPAYKRFQITWFVSWTPCPDCVVKVTEFLAEHPNVTLTISAARLYYYWETDYRRALCR

SEQ ID NO: 277 - protein, 108 aa, Artificial Sequence
  Synthetic
RRNTVWLCYKVKTKGPSRPPLNAKIFRGQVYFEPQYHAEMCFLSWFCGNQLSAYERFQITWFVSWTPCPDCVAMLAEFLAEHPNVTLTVSAARLYYYWERDYRGALRR

SEQ ID NO: 278 - protein, 108 aa, Artificial Sequence
  Synthetic
RRNTVWLCYEVKTRGPSMPTWGTKIFRGQVCFEPQYHAEMCFLSRFCGNQLPAYKRFQITWFVSWTPCPDCVAKVAEFLAEHPNVTLTISAARLYYYWETDYRRALCR

SEQ ID NO: 279 - protein, 108 aa, Artificial Sequence; identical to SEQ ID NO: 278
  Synthetic

SEQ ID NO: 280 - protein, 108 aa, Artificial Sequence
  Synthetic
RSYTWLCYEVKIKRGRSNLLWDTGVFRGQVYFEPQYHAEMCFLSWFCGNQLPAYKCFQITWFVSWTPCPDCVAKLAEFLSEHPNVTLTISAARLYYYWERDYRRALCR

SEQ ID NO: 281 - protein, 108 aa, Artificial Sequence
  Synthetic
RSYNWLCYEVKIKRGRSNLLWNTGVFRGQMYSQPEHHAEMCFLSWFCGNQLPAYKCFQITWFVSWTPCPDCVAKLAEFLAEYPNVTLTISAARLYYYWERDYRRALCR

SEQ ID NO: 282 - protein, 108 aa, Artificial Sequence
  Synthetic
RSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCR

SEQ ID NO: 283 - protein, 108 aa, Artificial Sequence
  Synthetic
RRNTVWLCYEVKTRGPSMPTWGTKIFRGQVYFQPQYHAEMCFLSRFCGNQLPAYKRFQITWFVSWNPCPDCVAKVIEFLAEHPNVTLTISAARLYYYWGRDWRRALRR

SEQ ID NO: 284 - protein, 102 aa, Artificial Sequence
  Synthetic
RRNTVWLCYKVKTRGPSMPTWGTKIFRGQVYFQPQYHAEMCFLSWFCGNQLPAYKRFQITWFVSWTPCPDCVVKVAEFLAEHPNVTLTISAARLYYYWETDY

SEQ ID NO: 285 - protein, 108 aa, Artificial Sequence
  Synthetic
RSYTWLCYEVKIKRGRSNLLWDTGVFRGQMYSQPEHHAEMCFLSWFCGNQLPAYKCFQITWFVSWTPCLDCVAKLAEFLAEYPNVTLTISTARLYYYWERDYRRALCR

SEQ ID NO: 286 - protein, 108 aa, Artificial Sequence
  Synthetic
RSYTWLCYEVKIRRGHSNLLWDTGVFRGQMYSQPEHHAEMYFLSWFCGNQLSAYKCFQITWFVSWTPCPDCVAKLAEFLAEHPNVTLTISAARLYYYWERDYRRALCR

SEQ ID NO: 287 - protein, 108 aa, Artificial Sequence
  Synthetic
RSYTWLCYEVKIRKDPSKLPWDTGVFRGQMYFQPEYHAEMCFLSWFCGNQLPAYKRFQITWFVSWTPCPDCVAKVAEFLAEHPNVTLTISAARLYYYWEKDWQRALCR

SEQ ID NO: 288 - protein, 108 aa, Artificial Sequence
  Synthetic
RRNTVWLCYEVKTKDPSRPRLDTKIFRGKVYFQLENHAEMCFLSWFCGNQLPANRCFQITWFVSWNPCLPCVAKVTKFLAEHPNVTLTISAARLYYYRARDWRRALRR

SEQ ID NO: 289 - protein, 108 aa, Artificial Sequence
  Synthetic
RSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCR

SEQ ID NO: 290 - protein, 108 aa, Artificial Sequence; identical to SEQ ID NO: 289
  Synthetic

SEQ ID NO: 291 - protein, 109 aa, Artificial Sequence
  Synthetic
RRSYTWLCYEVKIRKDPSKLPWDTGVFRGQMYFQPEYHAEMCFLSWFCGNQLPAYKRFQITWFVSWTPCPDCVAKVAVFLAEHPNVTLTISAARLYYYWEKDWQRALCR

SEQ ID NO: 292 - protein, 108 aa, Artificial Sequence
  Synthetic
RSYTWLCYEVKIRKDPSKLPWDTGVFRGQVYSEPEHHAEMYFLSWFCGNQLPAYKRFWITWFVSWTPCPDCVAKLAEFLTEHPNVTLTISAARLYYYRGRDWRRALCR

SEQ ID NO: 293 - protein, 108 aa, Artificial Sequence
  Synthetic
RSYTWLCYEVKKRKDPSKLPWDTGVFRGQVYSEPEHHAEMYFLSWFCGNQLPAYKRFWITWFVSWTPCPDCVAKVAEFLAEHPKVTLTISAARLYYYWDRDWRRALCR

SEQ ID NO: 294 - protein, 108 aa, Artificial Sequence
  Synthetic
RSYTWLCYEVKIRKDPSKLPWDTGVFRGQVYSEPEHHAEMYFLSWFCGNQLPAYKRFQITWFVSWTPCPDCVAKVAEFLTEHPNVTLTISAARLYYYRGRDWRRALCR

SEQ ID NO: 295 - protein, 108 aa, Artificial Sequence; identical to SEQ ID NO: 294
  Synthetic

SEQ ID NO: 296 - protein, 100 aa, Artificial Sequence
  Synthetic
RRNYTWLCYEVKIRKDPSKLAWDTGVFRGQVYSQPEHHAEMCFLSWFCGNQLSAYERFQITWFVSWTPCPDCVAKLAEFLAEHPNVTLTVSAARLYYYWE

SEQ ID NO: 297 - protein, 108 aa, Artificial Sequence
  Synthetic
RSYTWLCYEVKIRKDPSKLPWDTGVFRGQVYSKPEHHAEMCFLSRFCGNQLPAYKRFQITWFVSWNPCPDCVAKVIEFLAEHPNVTLTISTARLYYYWGRDWQRALCR

SEQ ID NO: 298 - protein, 108 aa, Artificial Sequence
  Synthetic
RSYTWLCYEVKIRKDPSKLPWDTGVFRGQVYSKPEHHAEMCFLSRFCGNQLPAYKRFQITWFVSWNPCPDCVVKVIEFLAEHPNVTLTISTARLYYYWGRDWQRALCR

SEQ ID NO: 299 - protein, 108 aa, Artificial Sequence
  Synthetic
RSYTWLCYEVKIRKDPSKLPWDTGVFRGQVYSKPEHHAEMCFLSRFCGNQLPAYKRFQITWFVSWNPCPDCVAKVTEFLAEHPNVTLTISTARLYYYWGRDWQRALCR

SEQ ID NO: 300 - protein, 108 aa, Artificial Sequence
  Synthetic
RSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCR

SEQ ID NO: 301 - protein, 121 aa, Artificial Sequence
  Synthetic
RRNYTWLCYEVKIRKDPSKLAWDTGVFRGQVLPKLQSNHRREVYFEPQYHAEMCFLSWFCGNQLSAYERFQITWFVSWTPCPDCVAMLAEFLAEHPNVTLTVSAARLYYYWERDYRGALRR

SEQ ID NO: 302 - protein, 120 aa, Artificial Sequence
  Synthetic
RRYTWLCYEVKIKKDPSKLPWDTGVFQGQVRPKFQSNRRYEVYFQPQYHAEMCFLSWFCGNQLPAYKHFQITWFVSWNPCPDCVAKVTEFLAEHPNVTLTISAARLYYYWGKDWRRALCR

SEQ ID NO: 303 - protein, 79 aa, Artificial Sequence
  Synthetic
MYSQPEHHAEMCFLSWFCGNQLSAYKCFQITWFVSWTPCPDCVAKLAKFLAEHPNVTLTISAARLYYYWERDYRRALCR

SEQ ID NO: 304 - protein, 120 aa, Artificial Sequence
  Synthetic
RSYTWLCYEVKIRKDPSKLPWDTGVFRGQVRPKLQSNRRYEVYFQPQYHAEMCFLSWFCGNQLPAYKRFQITWFVSWNPCPDCVAKVTEFLAEHPNVTLTISAARLYYYWGKDWRRALRR

SEQ ID NO: 305 - protein, 108 aa, Artificial Sequence
  Synthetic
RRYTWLCYEVKIMKDHSKLPWYTGVFRGQVYFEPQNHAEMCFLSWFCGNQLPAYECCQITWFVSWTPCPDCVAKVTEFLAEHPNVTLTISAARLYYYRGRDWRRALRR

SEQ ID NO: 306 - protein, 108 aa, Artificial Sequence
  Synthetic
RSYTWLCYEVKIRKDPSKLPWYTGVFRGQVYFQPQYHAEMCFLSWFCGNQLPANKRFQITWFVSWNPCPDCVAKVTEFLAEHPNVTLTISVARLYYYRGKDWRRALRR

SEQ ID NO: 307 - protein, 108 aa, Artificial Sequence
  Synthetic
RSYTWLCYEVKIRKDPSKLPWDTGVFRDQVYFQPQYHAEMCFLSWFCGNQLPANKRFQITWFVSWNPCPDCVTKVTEFLAEHPNVTLTISVARLYYYRGKDWRRALRR

SEQ ID NO: 308 - protein, 120 aa, Artificial Sequence
  Synthetic
RRYTWLCYEVKIKKDPSKLPWDTGVFPGQVRPKFQSNRRYEVYFQPQYHAEMYFLSWFCGNQLPAYKHFQITWFVSWNPCPDCVAKVTEFLAEHRNVTLTISAARLYYYWGKDWRRALCR

SEQ ID NO: 309 - protein, 129 aa, Artificial Sequence
  Synthetic
RSYTWLCYEVKIRKDPSKLPWDTGVFRGQVRPKLQSNRRYELSNWECRKHVYFQPQYHAEMCFLSWFCGNQLPANKRFQITWFVSWNPCPDCVAKVTEFLAEHPNVTLTISAARLYYYWGKDWRRALRR

SEQ ID NO: 310 - protein, 110 aa, Artificial Sequence
  Synthetic
GRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRL

SEQ ID NO: 311 - protein, 110 aa, Artificial Sequence
  Synthetic
GRSYTWLCYEVKIRRGHSNLLWDTGVFRGQMYSQPEHHAEMYFLSWFCGNQLPAYKCFQITWFVSWTPCPDCVAKLAEFLAEHPNVTLTISAARLYYYWERDYRRALCRL

SEQ ID NO: 312 - protein, 110 aa, Artificial Sequence
  Synthetic
GRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRL

SEQ ID NO: 313 - protein, 98 aa, Artificial Sequence
  Synthetic
RNTVWLCYEVKTKGPSRPPLDAKIFRGQVYFEPQYHAEMCFLSWFCGNQLPAYKCFQITWFVSWTPCPDCVAKLAEFLAEHPNVTLTISAARLYYYWE

SEQ ID NO: 314 - protein, 108 aa, Artificial Sequence
  Synthetic
RNTVWLCYEVKTKGPSRPRLDTKIFRGQVYFEPQYHAEMCFLSWFCGNQLPAYKCFQITWFVSWTPCPDCVAKLAEFLAEHPNVTLTISAARLYYYWERDYRRALCRL

SEQ ID NO: 315 - protein, 108 aa, Artificial Sequence
  Synthetic
RNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRL

SEQ ID NO: 316 - protein, 108 aa, Artificial Sequence
  Synthetic
RNTVWLCYEVKTRGPSMPTWGTKIFRGQVCFEPQYHAEMCFLSRFCGNQLPAYKRFQITWFVSWTPCPDCVAKVAEFLAEHPNVTLTISAARLYYYWETDYRRALCRL

SEQ ID NO: 317 - protein, 108 aa, Artificial Sequence; identical to SEQ ID NO: 316
  Synthetic

SEQ ID NO: 318 - protein, 108 aa, Artificial Sequence
  Synthetic
RNTVWLCYEVKTRGPSMPTWGAKIFRGQVYFEPQYHAEMCFLSWFCGNQLPAYKRFQITWFVSWTPCPDCVAKVAEFLAEHPNVTLTISAARLYYYWETDYRRALCRL

SEQ ID NO: 319 - protein, 101 aa, Artificial Sequence
  Synthetic
RNTVWLCYKVKTRGPSMPTWGTKIFRGQVYFQPQYHAEMCFLSWFCGNQLPAYKRFQITWFVSWTPCPDCVVKVAEFLAEHPNVTLTISAARLYYYWETDY

SEQ ID NO: 320 - protein, 108 aa, Artificial Sequence
  Synthetic
RNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRL

SEQ ID NO: 321 - protein, 108 aa, Artificial Sequence
  Synthetic
RNTVWLCYEVKTRGPSMPTWGTKIFRGQVYFQPQYHAEMCFLSRFCGNQLPAYKRFQITWFVSWNPCPDCVAKVIEFLAEHPNVTLTISAARLYYYWGRDWRRALRRL

SEQ ID NO: 322 - protein, 110 aa, Artificial Sequence
  Synthetic
GRSYTWLCYEVKIRKDPSKLPWYTGVFRGQVYSKPEHHAEMCFLSRFCGNQLPAYKRFQITWFVSWNPCPDCVAKVIEFLAEHPNVTLTISAARLYYYWSRDWQRALCRL

SEQ ID NO: 323 - protein, 110 aa, Artificial Sequence
  Synthetic
GRSYTWLCYEVKIRKDPSKLPWDTGVFRGQVYSKPEHHAEMCFLSRFCGNQLPAYKRFQITWFVSWNPCPDCVAKVIEFLAEHPNVTLTISTARLYYYWGRDWQRALCRL

SEQ ID NO: 324 - protein, 110 aa, Artificial Sequence; identical to SEQ ID NO: 323
  Synthetic

SEQ ID NO: 325 - protein, 110 aa, Artificial Sequence
  Synthetic
GRSYTWLCYEVKIRKDPSKLPWDTGVFRGQVYSKPEHHAEMCFLSRFCGNQLPAYKRFQITWFVSWNPCPDCVVKVIEFLAEHPNVTLTISTARLYYYWGRDWQRALCRL

SEQ ID NO: 326 - protein, 110 aa, Artificial Sequence
  Synthetic
GRSYTWLCYEVKIRKDPSKLPWYTGVFRGQVYSKPEHHAEMCFLSRFCGNQLPAYKRFQITWFVSWNPCPDCVAKVIEFLAEHPNVTLTIFTARLYYYWGRDWQRALCRL

SEQ ID NO: 327 - protein, 110 aa, Artificial Sequence
  Synthetic
GRSYTWLCYEVKIRKDPSKLPWDTGVFRGQVYSKPEHHAEMCFLSRFCGNQLPAYKRFQITWFVSWNPCPDCVAKVTEFLAEHPNVTLTISTARLYYYWGRDWQRALCRL

SEQ ID NO: 328 - protein, 110 aa, Artificial Sequence
  Synthetic
GRSYTWLCYEVKIRKDPSKLPWDTGVFRGQVYSEPEHHAEMYFLSWFCGNQLPAYKRFQITWFVSWTPCPDCVAKVAEFLTEHPNVTLTISAARLYYYRGRDWRRALCRL

SEQ ID NO: 329 - protein, 110 aa, Artificial Sequence; identical to SEQ ID NO: 328
  Synthetic

SEQ ID NO: 330 - protein, 110 aa, Artificial Sequence
  Synthetic
GRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRL

SEQ ID NO: 331 - protein, 109 aa, Artificial Sequence
  Synthetic
RSYTWLCYEVKIRKDPSKLPWDTGVFRGQMYFQPEYHAEMCFLSWFCGNQLPAYKRFQITWFVSWTPCPDCVAKVAVFLAEHPNVTLTISAARLYYYWEKDWQRALCRL

SEQ ID NO: 332 - protein, 110 aa, Artificial Sequence
  Synthetic
  misc_feature (103)..(103): Xaa can be any naturally occurring amino acid
GRSYTWLCYEVKIKKYPSKLLWDTGVFQGQVYFQPQYHAEMCFLSRFCGNQLPAYKRFQITWFVSWNPCPDCVAKVTEFLAEHPNVTLTISAARLYYYWEKDXRRALRRL

SEQ ID NO: 333 - protein, 110 aa, Artificial Sequence
  Synthetic
GRSYTWLCYEVKIKEDPSKLLWDTGVFQGQVYFQPQYHAEMCFLSRFCGNQLPAYKRFQITWFVSWNPCPDCVAKVTEFLAEHPNVTLTISAARLYYYWGRDWRRALRRL

SEQ ID NO: 334 - protein, 122 aa, Artificial Sequence
  Synthetic
GRRYTWLCYEVKIKKDPSKLPWDTGVFPGQVRPKFQSNRRYEVYFQPQYHAEMYFLSWFCGNQLPAYKHFQITWFVSWNPCPDCVAKVTEFLAEHRNVTLTISAARLYYYWGKDWRRALCRL

SEQ ID NO: 335 - protein, 106 aa, Artificial Sequence
  Synthetic
GRRYTWLCYEVKIKKDPSKLPWDTGVFPGQPQYHAEMYFLSWFCGNQLPAYKHFQITWFVSWNPCPDCVAKVTEFLAEHRNVTLTISAARLYYYWGKDWRRALCRL

SEQ ID NO: 336 - protein, 122 aa, Artificial Sequence
  Synthetic
GRRYTWLCYEVKIKKDPSKLPWDTGVFPGQVRPKFQSNRRQKVYFQPQYHAEMYFLSWFCGNQLPAYKHFQITWFVSWNPCPDCVAKVTEFLAEHRNVTLTISAARLYYYWGKDWRRALCRL

SEQ ID NO: 337 - protein, 122 aa, Artificial Sequence
  Synthetic
GRRYTWLCYEVKIKKDPSKLPWDTGVFQGQVRPKFQSNRRYEVYFQPQYHAEMCFLSWFCGNQLPAYKHFQITWFVSWNPCPDCVAKVTEFLAEHPNVTLTISAARLYYYWGKDWRRALCRL

SEQ ID NO: 338 - protein, 131 aa, Artificial Sequence
  Synthetic
GRSYTWLCYEVKIRKDPSKLPWDTGVFRGQVRPKLQSNRRYELSNWECRKRVYFQPQYHAEMYFLSWFCGNQLPANKRFQITWFASWNPCPDCVAKVTEFLAEHPNVTLTISVARLYYYRGKDWRRALRRL

SEQ ID NO: 339 - protein, 110 aa, Artificial Sequence
  Synthetic
GRSYTWLCYEVKIRKDPSKLPWDTGVFRGQVYFQPQYHAEMYFLSWFCGNQLPANKRFQITWFASWNPCPDCVAKVTEFLAEHPNVTLTISVARLYYYRGKDWRRALRRL

SEQ ID NO: 340 - protein, 110 aa, Artificial Sequence
  Synthetic
GRSYTWLCYEVKIRKDPSKLPWDTGVFRDQVYFQPQYHAEMCFLSWFCGNQLPANKRFQITWFVSWNPCPDCVTKVTEFLAEHPNVTLTISVARLYYYRGKDWRRALRRL

SEQ ID NO: 341 - protein, 110 aa, Artificial Sequence
  Synthetic
GRSYTWLCYEVKIRKDPSKLPWYTGVFRGQVYFQPQYHAEMCFLSWFCGNQLPANKRFQITWFVSWNPCPDCVAKVTEFLAEHPNVTLTISVARLYYYRGKDWRRALRRL

SEQ ID NO: 342 - protein, 110 aa, Artificial Sequence
  Synthetic
GRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRL

SEQ ID NO: 343 - protein, 122 aa, Artificial Sequence
  Synthetic
GRSYTWLCYEVKIRRGSSNLLWNTGVFRGPVPPKLQSNHRQEVYFQFENHAEMCFLSWFCGNRLPANRRFQITWFVSWNPCLPCVVKVTKFLAEHPNVTLTISAARLYYYRDREWRRVLRRL

SEQ ID NO: 344 - protein, 122 aa, Artificial Sequence
  Synthetic
GRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRL

SEQ ID NO: 345 - protein, 122 aa, Artificial Sequence; identical to SEQ ID NO: 344
  Synthetic

SEQ ID NO: 346 - protein, 122 aa, Artificial Sequence
  Synthetic
GRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRL

SEQ ID NO: 347 - protein, 122 aa, Artificial Sequence
  Synthetic
GRSYTWLCYEVKIRKDPSKLPWDKGVFRGQVLPKFQSNHRQEVYFQLENHAEMCFLSWFCGNQLPANRRFQITWFVSWNPCLPCVAKVTEFLAEHPNVTLTISAARLYYYRGRDWRRALRRL

SEQ ID NO: 348 - protein, 110 aa, Artificial Sequence
  Synthetic
GKKYTWLCYEVKIKKDTSKLPWNTGVFRGQVNFNPEHHAEMYFLSWFRGKLLPACKRSQITWFVSWNPCLYCVAKVAEFLAEHPNVTLTVSTARLYCYWKKDWRRALRKL

SEQ ID NO: 349 - protein, 110 aa, Artificial Sequence; identical to SEQ ID NO: 348
  Synthetic

SEQ ID NO: 350 - protein, 110 aa, Artificial Sequence
  Synthetic
GRRYTWLCYEVKIMKDHSKLPWYTGVFRGQVYFEPQNHAEMCFLSWFCGNQLPAYECCQITWFVSWTPCPDCVAKVTEFLAEHPNVTLTISAARLYYYRGRDWRRALRRL

SEQ ID NO: 351 - protein, 110 aa, Artificial Sequence
  Synthetic
GRRYTWLCYEVKISKDPSKLPWDTGIFRGQVYFEPQYHAEMCFLSWYCGNQLPAYKCFQITWFVSWTPCPDCVGKVAEFLAEHPNVTLTISAARLYYYWETDYRRALCRL

SEQ ID NO: 352 - protein, 121 aa, Artificial Sequence
  Synthetic
RNYTWLCYEVKIRKDPSKLAWDTGVFRGQVLPKLQSNHRREVYFEPQYHAEMCFLSWFCGNQLSAYERFQITWFVSWTPCPDCVAMLAEFLAEHPNVTLTVSAARLYYYWERDYRGALRRL

SEQ ID NO: 353 - RNA, 10 nt, Artificial Sequence
  Synthetic
gugaguacug

SEQ ID NO: 354 - RNA, 20 nt, Artificial Sequence
  Synthetic
agcgacacuugugaguacug

SEQ ID NO: 355 - RNA, 10 nt, Artificial Sequence
  Synthetic
ugaguacugu

SEQ ID NO: 356 - RNA, 20 nt, Artificial Sequence
  Synthetic
gcgacacuugugaguacugu

SEQ ID NO: 357 - RNA, 10 nt, Artificial Sequence
  Synthetic
uacucacaag

SEQ ID NO: 358 - RNA, 20 nt, Artificial Sequence
  Synthetic
uuucccacaguacucacaag

SEQ ID NO: 359 - RNA, 10 nt, Artificial Sequence
  Synthetic
caguucuuga

SEQ ID NO: 360 - RNA, 20 nt, Artificial Sequence
  Synthetic
gagauugcuacaguucuuga

SEQ ID NO: 361 - RNA, 10 nt, Artificial Sequence
  Synthetic
cacuguccac

SEQ ID NO: 362 - RNA, 20 nt, Artificial Sequence
  Synthetic
guagcaaucucacuguccac

SEQ ID NO: 363 - RNA, 10 nt, Artificial Sequence
  Synthetic
uguccacagg

SEQ ID NO: 364 - RNA, 20 nt, Artificial Sequence
  Synthetic
gcaaucucacuguccacagg

SEQ ID NO: 365 - RNA, 10 nt, Artificial Sequence
  Synthetic
ccacaggaga

SEQ ID NO: 366 - RNA, 20 nt, Artificial Sequence
  Synthetic
aucucacuguccacaggaga

SEQ ID NO: 367 - RNA, 10 nt, Artificial Sequence
  Synthetic
agauugcuac

SEQ ID NO: 368 - RNA, 20 nt, Artificial Sequence
  Synthetic
guggacagugagauugcuac

SEQ ID NO: 369 - RNA, 10 nt, Artificial Sequence
  Synthetic
agaagccaca

SEQ ID NO: 370 - RNA, 20 nt, Artificial Sequence
  Synthetic
uguccacaggagaagccaca

SEQ ID NO: 371 - RNA, 10 nt, Artificial Sequence
  Synthetic
acagugagau

SEQ ID NO: 372 - RNA, 20 nt, Artificial Sequence
  Synthetic
ucuccuguggacagugagau

SEQ ID NO: 373 - RNA, 10 nt, Artificial Sequence
  Synthetic
guggacagug

SEQ ID NO: 374 - RNA, 20 nt, Artificial Sequence
  Synthetic
ggcuucuccuguggacagug

SEQ ID NO: 375 - RNA, 10 nt, Artificial Sequence
  Synthetic
cuguggacag

SEQ ID NO: 376 - RNA, 20 nt, Artificial Sequence
  Synthetic
guggcuucuccuguggacag

SEQ ID NO: 377 - RNA, 10 nt, Artificial Sequence
  Synthetic
cacgggcgaa

SEQ ID NO: 378 - RNA, 20 nt, Artificial Sequence
  Synthetic
ggagaagccacacgggcgaa

SEQ ID NO: 379 - RNA, 10 nt, Artificial Sequence
  Synthetic
gcuucuccug

SEQ ID NO: 380 - RNA, 20 nt, Artificial Sequence
  Synthetic
cgcccguguggcuucuccug

SEQ ID NO: 381 - RNA, 10 nt, Artificial Sequence
  Synthetic
uggcuucucc

SEQ ID NO: 382 - RNA, 20 nt, Artificial Sequence
  Synthetic
uucgcccguguggcuucucc

SEQ ID NO: 383 - RNA, 10 nt, Artificial Sequence
  Synthetic
uucgcccgug

SEQ ID NO: 384 - RNA, 20 nt, Artificial Sequence
  Synthetic
uauaaggccuuucgcccgug

SEQ ID NO: 385 - RNA, 10 nt, Artificial Sequence
  Synthetic
aaaugcgagc

SEQ ID NO: 386 - RNA, 20 nt, Artificial Sequence
  Synthetic
aaggccuuauaaaugcgagc

SEQ ID NO: 387 - RNA, 10 nt, Artificial Sequence
  Synthetic
cgcauuuaua

SEQ ID NO: 388 - RNA, 20 nt, Artificial Sequence
  Synthetic
uugcacagcucgcauuuaua

SEQ ID NO: 389 - RNA, 10 nt, Artificial Sequence
  Synthetic
ugcccagagu

SEQ ID NO: 390 - RNA, 20 nt, Artificial Sequence
  Synthetic
acuaugccugugcccagagu

SEQ ID NO: 391 - RNA, 10 nt, Artificial Sequence
  Synthetic
uugcacagcu

SEQ ID NO: 392 - RNA, 20 nt, Artificial Sequence
  Synthetic
acaggcauaguugcacagcu

SEQ ID NO: 393 - RNA, 10 nt, Artificial Sequence
  Synthetic
cagaguagca

SEQ ID NO: 394 - RNA, 20 nt, Artificial Sequence
  Synthetic
ugccugugcccagaguagca

SEQ ID NO: 395 - RNA, 10 nt, Artificial Sequence
  Synthetic
auaguugcac

SEQ ID NO: 396 - RNA, 20 nt, Artificial Sequence
  Synthetic
gggcacaggcauaguugcac

SEQ ID NO: 397 - RNA, 10 nt, Artificial Sequence
  Synthetic
caagcucacc

SEQ ID NO: 398 - RNA, 20 nt, Artificial Sequence
  Synthetic
cccagaguagcaagcucacc

SEQ ID NO: 399 - RNA, 10 nt, Artificial Sequence
  Synthetic
gcacaggcau

SEQ ID NO: 400 - RNA, 20 nt, Artificial Sequence
  Synthetic
gcuacucugggcacaggcau

SEQ ID NO: 401 - RNA, 10 nt, Artificial Sequence
  Synthetic
cucugggcac

SEQ ID NO: 402 - RNA, 20 nt, Artificial Sequence
  Synthetic
gagcuugcuacucugggcac

SEQ ID NO: 403 - RNA, 10 nt, Artificial Sequence
  Synthetic
accaggcaca

SEQ ID NO: 404 - RNA, 20 nt, Artificial Sequence
  Synthetic
uagcaagcucaccaggcaca

SEQ ID NO: 405 - RNA, 10 nt, Artificial Sequence
  Synthetic
cacaugaaaa

SEQ ID NO: 406 - RNA, 20 nt, Artificial Sequence
  Synthetic
gcucaccaggcacaugaaaa

SEQ ID NO: 407 - RNA, 10 nt, Artificial Sequence
  Synthetic
cuugcuacuc

SEQ ID NO: 408 - RNA, 20 nt, Artificial Sequence
  Synthetic
gccuggugagcuugcuacuc

SEQ ID NO: 409 - RNA, 10 nt, Artificial Sequence
  Synthetic
caggugggga

SEQ ID NO: 410 - RNA, 20 nt, Artificial Sequence
  Synthetic
aacgcauggccaggugggga

SEQ ID NO: 411 - RNA, 10 nt, Artificial Sequence
  Synthetic
ggccaggugg

SEQ ID NO: 412 - RNA, 20 nt, Artificial Sequence
  Synthetic
gaaaacgcauggccaggugg

SEQ ID NO: 413 - RNA, 10 nt, Artificial Sequence
  Synthetic
agguggggaa

SEQ ID NO: 414 - RNA, 20 nt, Artificial Sequence
  Synthetic
acgcauggccagguggggaa

SEQ ID NO: 415 - RNA, 10 nt, Artificial Sequence
  Synthetic
uggggaagga

SEQ ID NO: 416 - RNA, 20 nt, Artificial Sequence
  Synthetic
cauggccagguggggaagga

SEQ ID NO: 417 - RNA, 10 nt, Artificial Sequence
  Synthetic
ccuggccaug

SEQ ID NO: 418 - RNA, 20 nt, Artificial Sequence
  Synthetic
uccuuccccaccuggccaug

SEQ ID NO: 419 - RNA, 10 nt, Artificial Sequence
  Synthetic
caccuggcca

SEQ ID NO: 420 - RNA, 20 nt, Artificial Sequence
  Synthetic
cguccuuccccaccuggcca

SEQ ID NO: 421 - RNA, 10 nt, Artificial Sequence
  Synthetic
uagcguguac

SEQ ID NO: 422 - RNA, 20 nt, Artificial Sequence
  Synthetic
agaugccuuuuagcguguac

SEQ ID NO: 423 - RNA, 10 nt, Artificial Sequence
  Synthetic
cacgcuaaaa

SEQ ID NO: 424 - RNA, 20 nt, Artificial Sequence
  Synthetic
ggguacuguacacgcuaaaa

SEQ ID NO: 425 - RNA, 10 nt, Artificial Sequence
  Synthetic
acacgcuaaa

SEQ ID NO: 426 - RNA, 20 nt, Artificial Sequence
  Synthetic
aggguacuguacacgcuaaa

SEQ ID NO: 427 - RNA, 10 nt, Artificial Sequence
  Synthetic
guacuguaca

SEQ ID NO: 428 - RNA, 20 nt, Artificial Sequence
  Synthetic
uuucuccaggguacuguaca

SEQ ID NO: 429 - RNA, 10 nt, Artificial Sequence
  Synthetic
uguugaauaa

SEQ ID NO: 430 - RNA, 20 nt, Artificial Sequence
  Synthetic
agugaucgaguguugaauaa

SEQ ID NO: 431 - RNA, 10 nt, Artificial Sequence
  Synthetic
gggcccgggc

SEQ ID NO: 432 - RNA, 20 nt, Artificial Sequence
  Synthetic
uuagugguccgggcccgggc

SEQ ID NO: 433 - RNA, 10 nt, Artificial Sequence
  Synthetic
gguccgggcc

SEQ ID NO: 434 - RNA, 20 nt, Artificial Sequence
  Synthetic
ccauauuagugguccgggcc

SEQ ID NO: 435 - RNA, 10 nt, Artificial Sequence
  Synthetic
auuagugguc

SEQ ID NO: 436 - RNA, 20 nt, Artificial Sequence
  Synthetic
cacgccccauauuagugguc

SEQ ID NO: 437 - RNA, 10 nt, Artificial Sequence
  Synthetic
gggcccgggc

SEQ ID NO: 438 - RNA, 20 nt, Artificial Sequence
  Synthetic
uuagugguccgggcccgggc

SEQ ID NO: 439 - RNA, 10 nt, Artificial Sequence
  Synthetic
gguccgggcc

SEQ ID NO: 440 - RNA, 20 nt, Artificial Sequence
  Synthetic
ccauauuagugguccgggcc

SEQ ID NO: 441 - RNA, 10 nt, Artificial Sequence
  Synthetic
auuagugguc

SEQ ID NO: 442 - RNA, 20 nt, Artificial Sequence
  Synthetic
cacgccccauauuagugguc

SEQ ID NO: 443 - RNA, 10 nt, Artificial Sequence
  Synthetic
cuguggacag

SEQ ID NO: 444 - RNA, 20 nt, Artificial Sequence
  Synthetic
guggcuucuccuguggacag

SEQ ID NO: 445 - RNA, 10 nt, Artificial Sequence
  Synthetic
uccuguggac

SEQ ID NO: 446 - RNA, 20 nt, Artificial Sequence
  Synthetic
guguggcuucuccuguggac

SEQ ID NO: 447 - RNA, 10 nt, Artificial Sequence
  Synthetic
cuucuccugu

SEQ ID NO: 448 - RNA, 20 nt, Artificial Sequence
  Synthetic
gcccguguggcuucuccugu

SEQ ID NO: 449 - RNA, 10 nt, Artificial Sequence
  Synthetic
uucgcccgug

SEQ ID NO: 450 - RNA, 20 nt, Artificial Sequence
  Synthetic
uauaaggccuuucgcccgug

SEQ ID NO: 451 - RNA, 10 nt, Artificial Sequence
  Synthetic
cuuucgcccg

SEQ ID NO: 452 - RNA, 20 nt, Artificial Sequence
  Synthetic
uuuauaaggccuuucgcccg

SEQ ID NO: 453 - RNA, 10 nt, Artificial Sequence
  Synthetic
gccuuucgcc

SEQ ID NO: 454 - RNA, 20 nt, Artificial Sequence
  Synthetic
cauuuauaaggccuuucgcc

SEQ ID NO: 455 - RNA, 10 nt, Artificial Sequence
  Synthetic
ugaguacugu

SEQ ID NO: 456 - RNA, 20 nt, Artificial Sequence
  Synthetic
gcgacacuugugaguacugu

SEQ ID NO: 457 - RNA, 10 nt, Artificial Sequence
  Synthetic
gugaguacug

SEQ ID NO: 458 - RNA, 20 nt, Artificial Sequence
  Synthetic
agcgacacuugugaguacug

SEQ ID NO: 459 - RNA, 10 nt, Artificial Sequence
  Synthetic
gcucaaaaga

SEQ ID NO: 460 - RNA, 20 nt, Artificial Sequence
  Synthetic
ggcaggcccagcucaaaaga

SEQ ID NO: 461 - RNA, 10 nt, Artificial Sequence
  Synthetic
ugaguacugu

SEQ ID NO: 462 - RNA, 20 nt, Artificial Sequence
  Synthetic
gcgacacuugugaguacugu

SEQ ID NO: 463 - RNA, 10 nt, Artificial Sequence
  Synthetic
uugugaguac

SEQ ID NO: 464 - RNA, 20 nt, Artificial Sequence
  Synthetic
gcagcgacacuugugaguac

SEQ ID NO: 465 - RNA, 10 nt, Artificial Sequence
  Synthetic
gacacuugug

SEQ ID NO: 466 - RNA, 20 nt, Artificial Sequence
  Synthetic
cagacgcagcgacacuugug

SEQ ID NO: 467 - RNA, 10 nt, Artificial Sequence
  Synthetic
acugugggaa

SEQ ID NO: 468 - RNA, 20 nt, Artificial Sequence
  Synthetic
acuugugaguacugugggaa

SEQ ID NO: 469 - RNA, 10 nt, Artificial Sequence
  Synthetic
gacacuugug

SEQ ID NO: 470 - RNA, 20 nt, Artificial Sequence
  Synthetic
cagacgcagcgacacuugug

SEQ ID NO: 471 - RNA, 10 nt, Artificial Sequence
  Synthetic
cagcgacacu

SEQ ID NO: 472 - RNA, 20 nt, Artificial Sequence
  Synthetic
agggcagacgcagcgacacu

SEQ ID NO: 473 - RNA, 10 nt, Artificial Sequence
  Synthetic
gcauuuauaa

SEQ ID NO: 474 - RNA, 20 nt, Artificial Sequence
  Synthetic
ugcacagcucgcauuuauaa

SEQ ID NO: 475 - RNA, 10 nt, Artificial Sequence
  Synthetic
cgcauuuaua

SEQ ID NO: 476 - RNA, 20 nt, Artificial Sequence
  Synthetic
uugcacagcucgcauuuaua

SEQ ID NO: 477 - RNA, 10 nt, Artificial Sequence
  Synthetic
ugaguacugu

SEQ ID NO: 478 - RNA, 20 nt, Artificial Sequence
  Synthetic
gcgacacuugugaguacugu

SEQ ID NO: 479 - RNA, 10 nt, Artificial Sequence
  Synthetic
gugaguacug

SEQ ID NO: 480 - RNA, 20 nt, Artificial Sequence
  Synthetic
agcgacacuugugaguacug

SEQ ID NO: 481 - RNA, 10 nt, Artificial Sequence
  Synthetic
uugcacagcu

SEQ ID NO: 482 - RNA, 20 nt, Artificial Sequence
  Synthetic
acaggcauaguugcacagcu

SEQ ID NO: 483 - RNA, 10 nt, Artificial Sequence
  Synthetic
auaguugcac

SEQ ID NO: 484 - RNA, 20 nt, Artificial Sequence
  Synthetic
gggcacaggcauaguugcac

SEQ ID NO: 485 - RNA, 10 nt, Artificial Sequence
  Synthetic
uugcacagcu

SEQ ID NO: 486 - RNA, 20 nt, Artificial Sequence
  Synthetic
acaggcauaguugcacagcu

SEQ ID NO: 487 - RNA, 10 nt, Artificial Sequence
  Synthetic
auaguugcac

SEQ ID NO: 488 - RNA, 20 nt, Artificial Sequence
  Synthetic
gggcacaggcauaguugcac

SEQ ID NO: 489 - RNA, 10 nt, Artificial Sequence
  Synthetic
uugcacagcu

SEQ ID NO: 490 - RNA, 20 nt, Artificial Sequence
  Synthetic
acaggcauaguugcacagcu

SEQ ID NO: 491 - RNA, 10 nt, Artificial Sequence
  Synthetic
auaguugcac

SEQ ID NO: 492 - RNA, 20 nt, Artificial Sequence
  Synthetic
gggcacaggcauaguugcac

SEQ ID NO: 493 - RNA, 10 nt, Artificial Sequence
  Synthetic
ugaguacugu

SEQ ID NO: 494 - RNA, 20 nt, Artificial Sequence
  Synthetic
gcgacacuugugaguacugu

SEQ ID NO: 495 - RNA, 10 nt, Artificial Sequence
  Synthetic
gugaguacug

SEQ ID NO: 496 - RNA, 20 nt, Artificial Sequence
  Synthetic
agcgacacuugugaguacug

SEQ ID NO: 497 - RNA, 10 nt, Artificial Sequence
  Synthetic
cucugggcac

SEQ ID NO: 498 - RNA, 20 nt, Artificial Sequence
  Synthetic
gagcuugcuacucugggcac

SEQ ID NO: 499 - RNA, 10 nt, Artificial Sequence
  Synthetic
uugcuacucu

SEQ ID NO: 500 - RNA, 20 nt, Artificial Sequence
  Synthetic
ccuggugagcuugcuacucu

SEQ ID NO: 501 - RNA, 10 nt, Artificial Sequence
  Synthetic
cuugcuacuc

SEQ ID NO: 502 - RNA, 20 nt, Artificial Sequence
  Synthetic
gccuggugagcuugcuacuc

SEQ ID NO: 503 - RNA, 10 nt, Artificial Sequence
  Synthetic
caggcauagu

SEQ ID NO: 504 - RNA, 20 nt, Artificial Sequence
  Synthetic
acucugggcacaggcauagu

SEQ ID NO: 505 - RNA, 10 nt, Artificial Sequence
  Synthetic
gcacaggcau

SEQ ID NO: 506 - RNA, 20 nt, Artificial Sequence
  Synthetic
gcuacucugggcacaggcau

SEQ ID NO: 507 - RNA, 10 nt, Artificial Sequence
  Synthetic
cucugggcac

SEQ ID NO: 508 - RNA, 20 nt, Artificial Sequence
  Synthetic
gagcuugcuacucugggcac

SEQ ID NO: 509 - RNA, 10 nt, Artificial Sequence
  Synthetic
uugcuacucu

SEQ ID NO: 510 - RNA, 20 nt, Artificial Sequence
  Synthetic
ccuggugagcuugcuacucu

SEQ ID NO: 511 - RNA, 10 nt, Artificial Sequence
  Synthetic
cuugcuacuc

SEQ ID NO: 512 - RNA, 20 nt, Artificial Sequence
  Synthetic
gccuggugagcuugcuacuc

SEQ ID NO: 513 - RNA, 10 nt, Artificial Sequence
  Synthetic
gcaaucucac

SEQ ID NO: 514 - RNA, 20 nt, Artificial Sequence
  Synthetic
aagaacuguagcaaucucac

SEQ ID NO: 515 - RNA, 10 nt, Artificial Sequence
  Synthetic
caagaacugu

SEQ ID NO: 516 - RNA, 20 nt, Artificial Sequence
  Synthetic
ggaaagucuucaagaacugu

SEQ ID NO: 517 - RNA, 10 nt, Artificial Sequence
  Synthetic
cuucaagaac

SEQ ID NO: 518 - RNA, 20 nt, Artificial Sequence
  Synthetic
gugggaaagucuucaagaac

SEQ ID NO: 519 - RNA, 10 nt, Artificial Sequence
  Synthetic
uugcuacucu

SEQ ID NO: 520 - RNA, 20 nt, Artificial Sequence
  Synthetic
ccuggugagcuugcuacucu

SEQ ID NO: 521 - RNA, 10 nt, Artificial Sequence
  Synthetic
cuugcuacuc

SEQ ID NO: 522 - RNA, 20 nt, Artificial Sequence
  Synthetic
gccuggugagcuugcuacuc

SEQ ID NO: 523 - RNA, 10 nt, Artificial Sequence
  Synthetic
uucaugugcc

SEQ ID NO: 524 - RNA, 20 nt, Artificial Sequence
  Synthetic
gccaugcguuuucaugugcc

SEQ ID NO: 525 - RNA, 10 nt, Artificial Sequence
  Synthetic
acgggcgaaa

SEQ ID NO: 526 - RNA, 20 nt, Artificial Sequence
  Synthetic
gagaagccacacgggcgaaa

SEQ ID NO: 527 - RNA, 10 nt, Artificial Sequence
  Synthetic
cacgggcgaa

SEQ ID NO: 528 - RNA, 20 nt, Artificial Sequence
  Synthetic
ggagaagccacacgggcgaa

SEQ ID NO: 529 - RNA, 10 nt, Artificial Sequence
  Synthetic
gccacacggg

SEQ ID NO: 530 - RNA, 20 nt, Artificial Sequence
  Synthetic
cacaggagaagccacacggg

SEQ ID NO: 531 - RNA, 10 nt, Artificial Sequence
  Synthetic
uucaugugcc

SEQ ID NO: 532 - RNA, 20 nt, Artificial Sequence
  Synthetic
gccaugcguuuucaugugcc

SEQ ID NO: 533 - RNA, 10 nt, Artificial Sequence
  Synthetic
cguuuucaug

SEQ ID NO: 534 - RNA, 20 nt, Artificial Sequence
  Synthetic
ccuggccaugcguuuucaug

SEQ ID NO: 535 - RNA, 10 nt, Artificial Sequence
  Synthetic
ugcguuuuca

SEQ ID NO: 536 - RNA, 20 nt, Artificial Sequence
  Synthetic
caccuggccaugcguuuuca

SEQ ID NO: 537 - RNA, 10 nt, Artificial Sequence
  Synthetic
acgggcgaaa

SEQ ID NO: 538 - RNA, 20 nt, Artificial Sequence
  Synthetic
gagaagccacacgggcgaaa

SEQ ID NO: 539 - RNA, 10 nt, Artificial Sequence
  Synthetic
cacgggcgaa

SEQ ID NO: 540 - RNA, 20 nt, Artificial Sequence
  Synthetic
ggagaagccacacgggcgaa

SEQ ID NO: 541 - RNA, 10 nt, Artificial Sequence
  Synthetic
uucaugugcc

SEQ ID NO: 542 - RNA, 20 nt, Artificial Sequence
  Synthetic
gccaugcguuuucaugugcc

SEQ ID NO: 543 - RNA, 10 nt, Artificial Sequence
  Synthetic
cguuuucaug

SEQ ID NO: 544 - RNA, 20 nt, Artificial Sequence
  Synthetic
ccuggccaugcguuuucaug

SEQ ID NO: 545 - RNA, 10 nt, Artificial Sequence
  Synthetic
ugcguuuuca

SEQ ID NO: 546 - RNA, 20 nt, Artificial Sequence
  Synthetic
caccuggccaugcguuuuca

SEQ ID NO: 547 - RNA, 10 nt, Artificial Sequence
  Synthetic
cacgggcgaa

SEQ ID NO: 548 - RNA, 20 nt, Artificial Sequence
  Synthetic
ggagaagccacacgggcgaa

SEQ ID NO: 549 - RNA, 10 nt, Artificial Sequence
  Synthetic
gaagccacac

SEQ ID NO: 550 - RNA, 20 nt, Artificial Sequence
  Synthetic
guccacaggagaagccacac

SEQ ID NO: 551 - RNA, 10 nt, Artificial Sequence
  Synthetic
agaagccaca

SEQ ID NO: 552 - RNA, 20 nt, Artificial Sequence
  Synthetic
uguccacaggagaagccaca

SEQ ID NO: 553 - RNA, 10 nt, Artificial Sequence
  Synthetic
ccuggccaug

SEQ ID NO: 554 - RNA, 20 nt, Artificial Sequence
  Synthetic
uccuuccccaccuggccaug

SEQ ID NO: 555 - RNA, 10 nt, Artificial Sequence
  Synthetic
caccuggcca

SEQ ID NO: 556 - RNA, 20 nt, Artificial Sequence
  Synthetic
cguccuuccccaccuggcca

SEQ ID NO: 557 - RNA, 10 nt, Artificial Sequence
  Synthetic
uuccccaccu

SEQ ID NO: 558 - RNA, 20 nt, Artificial Sequence
  Synthetic
guaaacguccuuccccaccu

SEQ ID NO: 559 - RNA, 10 nt, Artificial Sequence
  Synthetic
cuuccccacc

SEQ ID NO: 560 - RNA, 20 nt, Artificial Sequence
  Synthetic
uguaaacguccuuccccacc

SEQ ID NO: 561 - RNA, 10 nt, Artificial Sequence
  Synthetic
aaaugcgagc

SEQ ID NO: 562 - RNA, 20 nt, Artificial Sequence
  Synthetic
aaggccuuauaaaugcgagc

SEQ ID NO: 563 - RNA, 10 nt, Artificial Sequence
  Synthetic
uauaaaugcg

SEQ ID NO: 564 - RNA, 20 nt, Artificial Sequence
  Synthetic
cgaaaggccuuauaaaugcg

SEQ ID NO: 565 - RNA, 10 nt, Artificial Sequence
  Synthetic
cuuauaaaug

SEQ ID NO: 566 - RNA, 20 nt, Artificial Sequence
  Synthetic
ggcgaaaggccuuauaaaug

SEQ ID NO: 567 - RNA, 10 nt, Artificial Sequence
  Synthetic
ugugcaacua

SEQ ID NO: 568 - RNA, 20 nt, Artificial Sequence
  Synthetic
aaaugcgagcugugcaacua

SEQ ID NO: 569 - RNA, 10 nt, Artificial Sequence
  Synthetic
augcgagcug

SEQ ID NO: 570 - RNA, 20 nt, Artificial Sequence
  Synthetic
ggccuuauaaaugcgagcug

SEQ ID NO: 571 - RNA, 10 nt, Artificial Sequence
  Synthetic
aaaugcgagc

SEQ ID NO: 572 - RNA, 20 nt, Artificial Sequence
  Synthetic
aaggccuuauaaaugcgagc

SEQ ID NO: 573 - RNA, 10 nt, Artificial Sequence
  Synthetic
cuuccccacc

SEQ ID NO: 574 - RNA, 20 nt, Artificial Sequence
  Synthetic
uguaaacguccuuccccacc

SEQ ID NO: 575 - RNA, 10 nt, Artificial Sequence
  Synthetic
ugcccagagu

SEQ ID NO: 576 - RNA, 20 nt, Artificial Sequence
  Synthetic
acuaugccugugcccagagu

SEQ ID NO: 577 - RNA, 10 nt, Artificial Sequence
  Synthetic
cugugcccag

SEQ ID NO: 578 - RNA, 20 nt, Artificial Sequence
  Synthetic
gcaacuaugccugugcccag

SEQ ID NO: 579 - RNA, 10 nt, Artificial Sequence
  Synthetic
gccugugccc

SEQ ID NO: 580 - RNA, 20 nt, Artificial Sequence
  Synthetic
gugcaacuaugccugugccc

SEQ ID NO: 581 - RNA, 10 nt, Artificial Sequence
  Synthetic
ugcccagagu

SEQ ID NO: 582 - RNA, 20 nt, Artificial Sequence
  Synthetic
acuaugccugugcccagagu

SEQ ID NO: 583 - RNA, 10 nt, Artificial Sequence
  Synthetic
cugugcccag

SEQ ID NO: 584 - RNA, 20 nt, Artificial Sequence
  Synthetic
gcaacuaugccugugcccag

SEQ ID NO: 585 - RNA, 10 nt, Artificial Sequence
  Synthetic
gccugugccc

SEQ ID NO: 586 - RNA, 20 nt, Artificial Sequence
  Synthetic
gugcaacuaugccugugccc

SEQ ID NO: 587 - RNA, 10 nt, Artificial Sequence
  Synthetic
ugcccagagu

SEQ ID NO: 588 - RNA, 20 nt, Artificial Sequence
  Synthetic
acuaugccugugcccagagu

SEQ ID NO: 589 - RNA, 10 nt, Artificial Sequence
  Synthetic
cugugcccag

SEQ ID NO: 590 - RNA, 20 nt, Artificial Sequence
  Synthetic
gcaacuaugccugugcccag

SEQ ID NO: 591 - RNA, 10 nt, Artificial Sequence
  Synthetic
gccugugccc

SEQ ID NO: 592 - RNA, 20 nt, Artificial Sequence
  Synthetic
gugcaacuaugccugugccc

SEQ ID NO: 593 - RNA, 10 nt, Artificial Sequence
  Synthetic
cagaguagca

SEQ ID NO: 594 - RNA, 20 nt, Artificial Sequence
  Synthetic
ugccugugcccagaguagca

SEQ ID NO: 595 - RNA, 10 nt, Artificial Sequence
  Synthetic
ugcccagagu

SEQ ID NO: 596 - RNA, 20 nt, Artificial Sequence
  Synthetic
acuaugccugugcccagagu

SEQ ID NO: 597 - RNA, 10 nt, Artificial Sequence
  Synthetic
cugugcccag

SEQ ID NO: 598 - RNA, 20 nt, Artificial Sequence
  Synthetic
gcaacuaugccugugcccag

SEQ ID NO: 599 - RNA, 10 nt, Artificial Sequence
  Synthetic
cacgcuaaaa

SEQ ID NO: 600 - RNA, 20 nt, Artificial Sequence
  Synthetic
ggguacuguacacgcuaaaa

SEQ ID NO: 601 - RNA, 10 nt, Artificial Sequence
  Synthetic
acacgcuaaa

SEQ ID NO: 602 - RNA, 20 nt, Artificial Sequence
  Synthetic
aggguacuguacacgcuaaa

SEQ ID NO: 603 - RNA, 10 nt, Artificial Sequence
  Synthetic
cacgcuaaaa

SEQ ID NO: 604 - RNA, 20 nt, Artificial Sequence
  Synthetic
ggguacuguacacgcuaaaa

SEQ ID NO: 605 - RNA, 10 nt, Artificial Sequence
  Synthetic
acacgcuaaa

SEQ ID NO: 606 - RNA, 20 nt, Artificial Sequence
  Synthetic
aggguacuguacacgcuaaa

SEQ ID NO: 607 - RNA, 10 nt, Artificial Sequence
  Synthetic
uggggaagga

SEQ ID NO: 608 - RNA, 20 nt, Artificial Sequence
  Synthetic
cauggccagguggggaagga

SEQ ID NO: 609 - RNA, 10 nt, Artificial Sequence
  Synthetic
agguggggaa

SEQ ID NO: 610 - RNA, 20 nt, Artificial Sequence
  Synthetic
acgcauggccagguggggaa

SEQ ID NO: 611 - RNA, 10 nt, Artificial Sequence
  Synthetic
ggccaggugg

SEQ ID NO: 612 - RNA, 20 nt, Artificial Sequence
  Synthetic
gaaaacgcauggccaggugg

SEQ ID NO: 613 - RNA, 10 nt, Artificial Sequence
  Synthetic
ucgaucacug

SEQ ID NO: 614 - RNA, 20 nt, Artificial Sequence
  Synthetic
uauucaacacucgaucacug

SEQ ID NO: 615 - RNA, 10 nt, Artificial Sequence
  Synthetic
acucgaucac

SEQ ID NO: 616 - RNA, 20 nt, Artificial Sequence
  Synthetic
auuauucaacacucgaucac

SEQ ID NO: 617 - RNA, 10 nt, Artificial Sequence
  Synthetic
ucgaucacug

SEQ ID NO: 618 - RNA, 20 nt, Artificial Sequence
  Synthetic
uauucaacacucgaucacug

SEQ ID NO: 619 - RNA, 10 nt, Artificial Sequence
  Synthetic
acucgaucac

SEQ ID NO: 620 - RNA, 20 nt, Artificial Sequence
  Synthetic
auuauucaacacucgaucac

SEQ ID NO: 621 - RNA, 10 nt, Artificial Sequence
  Synthetic
auucaacacu

SEQ ID NO: 622 - RNA, 20 nt, Artificial Sequence
  Synthetic
uuauaucauuauucaacacu

SEQ ID NO: 623 - RNA, 10 nt, Artificial Sequence
  Synthetic
aguacccugg

SEQ ID NO: 624 - RNA, 20 nt, Artificial Sequence
  Synthetic
uagcguguacaguacccugg

SEQ ID NO: 625 - RNA, 10 nt, Artificial Sequence
  Synthetic
acaguacccu

SEQ ID NO: 626 - RNA, 20 nt, Artificial Sequence
  Synthetic
uuuagcguguacaguacccu

SEQ ID NO: 627 - RNA, 10 nt, Artificial Sequence
  Synthetic
uacaguaccc

SEQ ID NO: 628 - RNA, 20 nt, Artificial Sequence
  Synthetic
uuuuagcguguacaguaccc

(19) United States
(12) Patent Application Publication
(10) Pub. No.: US 2024/0189457 A1
(43) Pub. Date: Jun. 13, 2024

(54) GENE THERAPY FOR TREATING BETA-HEMOGLOBINOPATHIES

(71) Applicant: ShanghaiTech University, Shanghai (CN)

(72) Inventors: Jia CHEN, Shanghai (CN); Bei YANG, Shanghai (CN); Li YANG, Shanghai (CN); Wenyan HAN, Shanghai (CN); Shangwu SUN, Shanghai (CN); Ying ZHANG, Shanghai (CN)

(21) Appl. No.: 18/553,729

(22) PCT Filed: Apr. 2, 2022
(86) PCT No.: PCT/CN2022/084982

§ 371 Date: Oct. 2, 2023

(30) Foreign Application Priority Data
Apr. 2, 2021 (WO) PCT/CN2021/085285
Aug. 27, 2021 (WO) PCT/CN2021/115140

(51) Int. Cl. A61K 48/00; A61K 38/17; C12N 5/078; C12N 5/0789; C12N 9/22; C12N 9/78; C12N 15/11

(52) U.S. Cl. CPC A61K 48/005; A61K 38/1722; C12N 5/0641; C12N 5/0647; C12N 9/22; C12N 9/78; C12N 15/111; C07K 2319/50; C12N 2310/20; C12Y 305/04005

(57) ABSTRACT

Provided are gene therapy technologies, including specifically designed and tested guide RNA sequences for improved base editors, useful for increasing the expression of the gamma-globin gene. The guide RNA sequences may target the BCL11A erythroid enhancer or the gamma-globin promoter, or both at the same time. The base editors can include nucleobase deaminase inhibitor that inhibits the editing activity of the base editors until they are bound to the target sites. These gene therapy technologies are useful for treating diseases including beta-thalassemia and sickle cell anemia, among others.

### BACKGROUND

Beta-hemoglobinopathies, including sickle cell disease (SCD) and β-thalassemia, can be caused by genetic mutations in the β-globin gene (HBB). In addition to hematopoietic stem cell transplantation, gene therapy is one of the most promising treatments for these diseases. Gene therapy is a therapeutic strategy of human hereditary diseases, through gene addition or genome editing to treat hereditary diseases.

Previously, it was found that a few patients with naturally existing mutations in the HBB gene cluster or related genes maintain high γ-globin expression from their childhood to adulthood and do not show serious symptoms of anemia. This suggests that a therapeutic strategy for β-hemoglobinopathies is through reactivation of the expression of γ-globin. Currently, there are two common strategies to reactivate the expression of γ-globin. The first is to knock down a gene (e.g., BCL11A) that suppresses the expression of γ-globin gene (HBG1/2) and the second is to disrupt the binding sequences of transcription factors at the promoters of the HBG1/2 genes. Different methods have been tested to implement the aforementioned therapeutic strategies, such as CRISPR/Cas (Clustered regularly interspaced short palindromic repeats/CRISPR-associated protein)-induced nucleotide insertions/deletions (indels) or base editor (BE)-induced point mutations.

CRISPR/Cas system has been the most prevalent genomic editing tool because of its convenience and high editing efficiency in living organisms. Directed by a guide RNA, the Cas nuclease can generate DNA double strand breaks (DSBs) at the targeted genomic sites in various cells (both cell lines and cells from living organisms). These DSBs are then repaired by the endogenous DNA repair system, which could be utilized to perform desired genome editing.

In general, two major DNA repair pathways can be activated by DSBs, non-homologous end joining (NHEJ) and homology-directed repair (HDR). NHEJ can introduce random indels in the genomic DNA region around the DSBs, thereby leading to open reading frame (ORF) shift and ultimately gene inactivation. In contrast, when HDR is triggered, the genomic DNA sequence at target site could be replaced by the sequence of the exogenous donor DNA through a homologous recombination mechanism, which can be used to induce base substitutions. However, the practical efficiency of HDR-mediated base substitution is low (normally <5%) because the occurrence of homologous recombination is both cell type-specific and cell cycle-dependent and NHEJ is triggered more frequently than HDR.

Base editor (BE), which combines the CRISPR/Cas system with the APOBEC (apolipoprotein B mRNA editing enzyme, catalytic polypeptide-like) cytosine deaminase family members, was recently developed to induce base substitutions with high efficiency. Through the fusion with Cas9 nickase (nCas9) or catalytically dead Cpf1 (dCpf1, also known as dCas12a), the cytosine (C) deaminase activity of APOBEC/AID family members can be purposely directed to the target genomic sites to induce C to Thymine (T) substitutions.

The safety and efficiency of gene editing are of great importance in gene therapy. Previous studies have reported that the DSBs induced by Cas9 nuclease can activate a p53-mediated DNA damage response pathway and then lead to cell death. Moreover, APOBEC/AID family members can trigger C-to-T base substitutions in single-stranded DNA (ssDNA) regions, which are formed randomly during various cellular processes including DNA replication, repair and transcription. Thus, the specificity of previous base editing systems is compromised, limiting the applications of BEs for therapeutic purposes.

### SUMMARY

The instant disclosure, in some embodiments, describes improved gene therapy technologies useful for increasing the production of the γ-globin gene, which is useful for treating various hematological diseases, in particular inherited ones, such as beta-thalassemia and sickle cell anemia. Using a newly designed base editor, referred to as a transformer Base Editor (tBE), the present technology employs specifically designed guide RNA sequences to target the BCL11A erythroid enhancer or the C-terminal three tandem C2H2 zinc fingers (Znf4˜6) for inactivation, or to the γ-globin promoter for activation. Such a base editor has improved efficiency and specificity, as demonstrated in the experimental examples.

In accordance with one embodiment of the present disclosure, provided is a base editing system, or one or more polynucleotides encoding the base editing system. In some embodiments, the base editing system comprises a CRISPR-associated (Cas) protein, a nucleobase deaminase, a single-guide RNA (sgRNA), and a helper single-guide RNA (hsgRNA). The sgRNA and the hsgRNA target sites at the BCL11A erythroid enhancer. In some embodiments, the sgRNA and the hsgRNA target sites at the BCL11A-binding motif in the γ-globin promoter. In some embodiments, the sgRNA and the hsgRNA target sites at the ZBTB7A-binding motif in the γ-globin promoter. In some embodiments, the sgRNA and the hsgRNA target sites at a GATA1-half-E-box motif of BCL11A. In some embodiments, the sgRNA and the hsgRNA target sites at KLF1-binding motifs of BCL11A. In some embodiments, the sgRNA and the hsgRNA target sites at a GATA1-binding motif of NFIX. In some embodiments, the sgRNA and the hsgRNA target sites at the coding sequences of Znf4˜6 in BCL11A. Example sgRNA and hsgRNA sequences are provided in Tables 1-11.

In one embodiment, provided is a method for promoting production of γ-globin in a human cell, comprising introducing into the cell a CRISPR-associated (Cas) protein, a nucleobase deaminase, a single-guide RNA (sgRNA), and a helper single-guide RNA (hsgRNA), wherein (a) the sgRNA comprises a nucleic acid sequence selected from the group consisting of SEQ ID NO:1-10, and the hsgRNA comprises a nucleic acid sequence selected from the group consisting of SEQ ID NO:11-28; (b) the sgRNA comprises the nucleic acid sequence of SEQ ID NO:29-30, and the hsgRNA comprises a nucleic acid sequence selected from the group consisting of SEQ ID NO:31-36, (c) the sgRNA comprises a nucleic acid sequence selected from the group consisting of SEQ ID NO:37-54, and the hsgRNA comprises a nucleic acid sequence selected from the group consisting of SEQ ID NO:63-116, (d) the sgRNA comprises a nucleic acid sequence selected from the group consisting of SEQ ID NO: 117-122, and the hsgRNA comprises a nucleic acid sequence selected from the group consisting of SEQ ID NO:123-138, or (e) the sgRNA comprises a nucleic acid sequence selected from the group consisting of SEQ ID NO:139-150, and the hsgRNA comprises a nucleic acid sequence selected from the group consisting of SEQ ID NO:151-190, (f) the sgRNA comprises a nucleic acid sequence selected from the group consisting of SEQ ID NO:353-430, and the hsgRNA comprises a nucleic acid sequence selected from the group consisting of SEQ ID NO:431-628, In some embodiments, the Cas protein, the nucleobase deaminase, the sgRNA, and the hsgRNA are preferably introduced into the cell by one or more encoding polynucleotides.

In some embodiments, (a) the sgRNA comprises a nucleic acid sequence selected from the group consisting of SEQ ID NO:1-10, and the hsgRNA comprises a nucleic acid sequence selected from the group consisting of SEQ ID NO:11-28; (b) the sgRNA comprises the nucleic acid sequence of SEQ ID NO:29-30, and the hsgRNA comprises a nucleic acid sequence selected from the group consisting of SEQ ID NO:31-36; (c) the sgRNA comprises a nucleic acid sequence selected from the group consisting of SEQ ID NO:37-54, and the hsgRNA comprises a nucleic acid sequence selected from the group consisting of SEQ ID NO:63-116; (d) the sgRNA comprises a nucleic acid sequence selected from the group consisting of SEQ ID NO: 118-122, and the hsgRNA comprises a nucleic acid sequence selected from the group consisting of SEQ ID NO:123-138; or (e) the sgRNA comprises a nucleic acid sequence selected from the group consisting of SEQ ID NO:139-150, and the hsgRNA comprises a nucleic acid sequence selected from the group consisting of SEQ ID NO:151-190. In some embodiments, the sgRNA comprises the nucleic acid sequence of SEQ ID NO:4, and the hsgRNA comprises the nucleic acid of SEQ ID NO:11. In some embodiments, the sgRNA comprises the nucleic acid sequence of SEQ ID NO:4, and the hsgRNA comprises the nucleic acid of SEQ ID NO:12.

In some embodiments, the sgRNA comprises the nucleic acid sequence of SEQ ID NO:30, and the hsgRNA comprises a nucleic acid sequence selected from the group consisting of SEQ ID NO:31-36, preferably SEQ ID NO:33 or 34. In some embodiments, both sets of sgRNA/hsgRNA are included.

In some embodiments, the nucleobase deaminase is a cytidine deaminase. Non-limiting examples include APOBEC3B (A3B), APOBEC3C (A3C), APOBEC3D (A3D), APOBEC3F (A3F), APOBEC3G (A3G), APOBEC3H (A3H), APOBEC1 (A1), APOBEC3 (A3), APOBEC2 (A2), APOBEC4 (A4) and AICDA (AID).

In some embodiments, the base editing system further comprises a nucleobase deaminase inhibitor, fused to the nucleobase deaminase, via a protease cleavage site. In some embodiments, the nucleobase deaminase inhibitor is an inhibitory domain of a nucleobase deaminase. In some embodiments, the nucleobase deaminase inhibitor is an inhibitory domain of a cytidine deaminase. Non-limiting examples include SEQ ID NO: 192-193.

In some embodiments, the base editing system further comprises a protease that is capable of cleaving at the protease cleavage site. In some embodiments, the protease is selected from the group consisting of TuMV protease, PPV protease, PVY protease, ZIKV protease and WNV protease. In some embodiments, the protease cleaves the cleavage site only when the base editor is at the target site determined by the guide RNAs.

In some embodiments, the Cas protein is selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

In some embodiments, the Cas protein is catalytically impaired, such as nCas9 and dCpf1.

Also provided, in one embodiment, is a method of using the base editors, or one or more polynucleotides that encode the base editors, to promote production of γ-globin in a human cell, which may be an erythroid cell, a hematopoietic stem cell, or a stem cell, among others. In some embodiments, the method is carried out ex vivo or in vivo in a patient. In some embodiments, the patient suffers from 0-thalassemia, sickle cell anemia, Haemoglobin C, or Haemoglobin E.

Yet another embodiment provides base editors that incorporate a cytidine deaminase inhibitor. Examples include hA3F-CDA1 and its analogs. In some embodiments, a fusion protein is provided, comprising: a first fragment comprising a cytidine deaminase or a catalytic domain thereof, a second fragment comprising a cytidine deaminase inhibitor comprising an amino acid sequence selected from the group consisting of SEQ ID NO:192, and 265-309 and sequences having at least 85% sequence identity to any of SEQ ID NO:192, and 265-309, and a protease cleavage site between the first fragment and the second fragment. Also provided are methods of using such fusion proteins for base editing and treatments.

### BRIEF DESCRIPTION OF THE DRAWINGS

FIG. 1: Editing efficiencies induced by tBE with the pairs of sgRNA-BCL11A-2 and its hsgRNAs targeting BCL11A erythroid enhancer region. FIG. 1A: Schematic diagram illustrating the co-transfection of the plasmids for expressing tBE system. One plasmid expresses SpD10A nickase and another expresses sgRNA-BCL11A-2, different hsgRNA-BCL11As and cytidine deaminase complex of tBE. FIG. 1B: Sanger sequencing results show the base editing efficiencies induced by tBE with indicated pairs of sgRNA/hsgRNA and YE1-BE4max with indicated sgRNA targeting BCL11A erythroid enhancer region.

FIG. 2: Editing efficiencies induced by tBE with the pairs of sgRNA-BCL11A-3 and its hsgRNAs targeting BCL11A erythroid enhancer region. FIG. 2A: Schematic diagram illustrating the co-transfection of the plasmids for expressing tBE system. One plasmid expresses SpD10A nickase and another expresses sgRNA-BCL11A-3, different hsgRNA-BCL11As and cytidine deaminase complex of tBE. FIG. 2B: Sanger sequencing results show the base editing efficiencies induced by tBE with indicated pairs of sgRNA/hsgRNA and YE1-BE4max with indicated sgRNA targeting BCL11A erythroid enhancer region.

FIG. 3: Editing efficiencies induced by tBE with the pairs of sgRNA-BCL11A-4 and its hsgRNAs targeting BCL11A erythroid enhancer region. FIG. 3A: Schematic diagram illustrating the co-transfection of the plasmids for expressing tBE system. One plasmid expresses SpD10A nickase and another expresses sgRNA-BCL11A-4, different hsgRNA-BCL11As and cytidine deaminase complex of tBE. FIG. 3B: Sanger sequencing results show the base editing efficiencies induced by tBE with indicated pairs of sgRNA/hsgRNA and YE1-BE4max with indicated sgRNA targeting BCL11A erythroid enhancer region.

FIG. 4: Editing efficiencies induced by tBE with the pairs of sgRNA-BCL11A-5 and its hsgRNAs targeting BCL11A erythroid enhancer region. FIG. 4A: Schematic diagram illustrating the co-transfection of the plasmids for expressing tBE system. One plasmid expresses SpD10A nickase and another expresses sgRNA-BCL11A-5, different hsgRNA-BCL11As and cytidine deaminase complex of tBE. FIG. 4B: Sanger sequencing results show the base editing efficiencies induced by tBE with indicated pairs of sgRNA/hsgRNA and YE1-BE4max with indicated sgRNA targeting BCL11A erythroid enhancer region.

FIG. 5: Editing efficiencies induced by tBE with the pairs of sgRNA-HBG and its hsgRNAs targeting the core binding sites of transcription factors in the promoters regions of γ-globin gene (HBG1/2). FIG. 5A: Schematic diagram illustrating the co-transfection of the plasmids for expressing tBE system. One plasmid expresses SpD10A nickase and another expresses sgRNA-HBG, hsgRNA-HBG and cytidine deaminase complex of tBE. FIG. 5B: Sanger sequencing results show the base editing efficiencies induced by tBE with indicated pairs of sgRNA/hsgRNA and YE1-BE4max with indicated sgRNA targeting the core binding sites of transcription factors in the promoter regions of γ-globin gene (HBG1/2).

FIG. 6: Editing efficiencies induced by tBE with the pairs of sgRNA-HBG/hsgRNA-HBG-1 targeting the core binding sites of transcription factors in the promoter regions of γ-globin gene (HBG1/2), and the pairs of sgRNA-BCL11A-3/hsgRNA-BCL11A-1 targeting BCL11A erythroid enhancer region simultaneously. FIG. 6A: Schematic diagram illustrating the co-transfection of the plasmids for expressing tBE system to edit HBG1/2 promoters and BCL11A erythroid enhancer regions simultaneously. FIG. 6B: Sanger sequencing results show the base editing efficiencies induced by tBE with two indicated pairs of sgRNA/hsgRNA targeting the core binding sites of transcription factors in the promoter regions of 7-globin gene (HBG1/2) and the BCL11A erythroid enhancer region simultaneously.

FIG. 7: Editing efficiencies induced by tBE with the pairs of sgRNA-HBG/hsgRNA-HBG-2 targeting the core binding sites of transcription factors in the promoter regions of γ-globin gene (HBG1/2), and the pairs of sgRNA-BCL11A-3/hsgRNA-BCL11A-1 targeting BCL11A erythroid enhancer region simultaneously. FIG. 7A: Schematic diagram illustrating the co-transfection of the plasmids for expressing tBE system to edit HBG1/2 promoters and BCL11A erythroid enhancer regions simultaneously. FIG. 7B: Sanger sequencing results show the base editing efficiencies induced by tBE with two indicated pairs of sgRNA/hsgRNA targeting the core binding sites of transcription factors in the promoters regions of 7-globin gene (HBG1/2) and the BCL11A erythroid enhancer region simultaneously.

FIG. 8: Editing efficiencies induced by tBE with the pairs of sgRNA-HBG/hsgRNA-HBG-3 targeting the core binding sites of transcription factors in the promoter regions of γ-globin gene (HBG1/2), and the pairs of sgRNA-BCL11A-3/hsgRNA-BCL11A-1 targeting BCL11A erythroid enhancer region simultaneously. FIG. 8A: Schematic diagram illustrating the co-transfection of the plasmids for expressing tBE system to edit HBG1/2 promoter and BCL11A erythroid enhancer regions simultaneously. FIG. 8B: Sanger sequencing results show the base editing efficiencies induced by tBE with two indicated pairs of sgRNA/hsgRNA targeting the core binding sites of transcription factors in the promoters regions of 7-globin gene (HBG1/2) and the BCL11A erythroid enhancer region simultaneously.

FIG. 9: Editing efficiencies induced by tBE with the pairs of sgRNA-HBG/hsgRNA-HBG-1 targeting the core binding sites of transcription factors in the promoter regions of γ-globin gene (HBG1/2), and the pairs of sgRNA-BCL11A-3/hsgRNA-BCL11A-2 targeting BCL11A erythroid enhancer region simultaneously. FIG. 9A: Schematic diagram illustrating the co-transfection of the plasmids for expressing tBE system to edit HBG1/2 promoter and BCL11A erythroid enhancer regions simultaneously. FIG. 9B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 10: Editing efficiencies induced by tBE with the pairs of sgRNA-HBG/hsgRNA-HBG-2 targeting the core binding sites of transcription factors in the promoter regions of γ-globin gene (HBG1/2), and the pairs of sgRNA-BCL11A-3/hsgRNA-BCL11A-2 targeting BCL11A erythroid enhancer region simultaneously. FIG. 10A: Schematic diagram illustrating the co-transfection of the plasmids for expressing tBE system to edit HBG1/2 promoters and BCL11A erythroid enhancer regions simultaneously. FIG. 10B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 11: Editing efficiencies induced by tBE with the pairs of sgRNA-HBG/hsgRNA-HBG-3 targeting the core binding sites of transcription factors in the promoter regions of γ-globin gene (HBG1/2), and the pairs of sgRNA-BCL11A-3/hsgRNA-BCL11A-2 targeting BCL11A erythroid enhancer region simultaneously. FIG. 11A: Schematic diagram illustrating the co-transfection of the plasmids for expressing tBE system to edit HBG1/2 promoters and BCL11A erythroid enhancer regions simultaneously. FIG. 11B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 12: Editing efficiencies induced by tBE with the pairs of sgRNA-HBG/hsgRNA-HBG-1 targeting the core binding sites of transcription factors in the promoter regions of γ-globin gene (HBG1/2), and the pairs of sgRNA-BCL11A-4/hsgRNA-BCL11A-1 targeting BCL11A erythroid enhancer region simultaneously. FIG. 12A: Schematic diagram illustrating the co-transfection of the plasmids for expressing tBE system to edit HBG1/2 promoters and BCL11A erythroid enhancer regions simultaneously. FIG. 12B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 13: Editing efficiencies induced by tBE with the pairs of sgRNA-HBG/hsgRNA-HBG-2 targeting the core binding sites of transcription factors in the promoter regions of γ-globin gene (HBG1/2), and the pairs of sgRNA-BCL11A-4/hsgRNA-BCL11A-1 targeting BCL11A erythroid enhancer region simultaneously. FIG. 13A: Schematic diagram illustrating the co-transfection of the plasmids for expressing tBE system to edit HBG1/2 promoters and BCL11A erythroid enhancer regions simultaneously. FIG. 13B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 14: Editing efficiencies induced by tBE with the pairs of sgRNA-HBG/hsgRNA-HBG-3 targeting the core binding sites of transcription factors in the promoter regions of γ-globin gene (HBG1/2), and the pairs of sgRNA-BCL11A-4/hsgRNA-BCL11A-1 targeting BCL11A erythroid enhancer region simultaneously. FIG. 14A: Schematic diagram illustrating the co-transfection of the plasmids for expressing tBE system to edit HBG1/2 promoters and BCL11A erythroid enhancer regions simultaneously. FIG. 14B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 15: Editing efficiencies induced by tBE with the pairs of sgRNA-HBG/hsgRNA-HBG-1 targeting the core binding sites of transcription factors in the promoter regions of γ-globin gene (HBG1/2), and the pairs of sgRNA-BCL11A-4/hsgRNA-BCL11A-2 targeting BCL11A erythroid enhancer region simultaneously. FIG. 15A: Schematic diagram illustrating the co-transfection of the plasmids for expressing tBE system to edit HBG1/2 promoters and BCL11A erythroid enhancer regions simultaneously. FIG. 15B: Sanger sequencing results show the base editing efficiencies induced by tBE with two indicated pairs of sgRNA/hsgRNA targeting the core binding sites of transcription factors in the promoters regions of γ-globin gene (HBG1/2) and the BCL11A erythroid enhancer region simultaneously.

FIG. 16: Editing efficiencies induced by tBE with the pairs of sgRNA-HBG/hsgRNA-HBG-2 targeting the core binding sites of transcription factors in the promoter regions of 7-globin gene (HBG1/2), and the pairs of sgRNA-BCL11A-4/hsgRNA-BCL11A-2 targeting BCL11A erythroid enhancer region simultaneously. FIG. 16A: Schematic diagram illustrating the co-transfection of the plasmids for expressing tBE system to edit HBG1/2 promoters and BCL11A erythroid enhancer regions simultaneously. FIG. 16B: Sanger sequencing results show the base editing efficiencies induced by tBE with two indicated pairs of sgRNA/hsgRNA targeting the core binding sites of transcription factors in the promoters regions of 7-globin gene (HBG1/2) and the BCL11A erythroid enhancer region simultaneously.

FIG. 17: Editing efficiencies induced by tBE with the pairs of sgRNA-HBG/hsgRNA-HBG-3 targeting the core binding sites of transcription factors in the promoter regions of 7-globin gene (HBG1/2), and the pairs of sgRNA-BCL11A-4/hsgRNA-BCL11A-2 targeting BCL11A erythroid enhancer region simultaneously. FIG. 17A: Schematic diagram illustrating the co-transfection of the plasmids for expressing tBE system to edit HBG1/2 promoters and BCL11A erythroid enhancer regions simultaneously. FIG. 17B: Sanger sequencing results show the base editing efficiencies induced by tBE with two indicated pairs of sgRNA/hsgRNA targeting the core binding sites of transcription factors in the promoters regions of 7-globin gene (HBG1/2) and the BCL11A erythroid enhancer region simultaneously.

FIG. 18: Editing efficiencies induced by tBE with the pairs of sgRNA-KLF1-1-1 and its hsgRNAs targeting one of the three KLF1 binding motifs of BCL11A locates in +55 kb DHS of BCL11A erythroid enhancer region. FIG. 18A: Schematic diagram illustrating the co-transfection of the plasmids for expressing tBE system. One plasmid expresses SpD10A nickase and another expresses sgRNA-KLF1-1-1, hsgRNA-KLF1 and cytidine deaminase complex of tBE. FIG. 18B: Sanger sequencing results show the base editing efficiencies induced by tBE with indicated pairs of sgRNA/hsgRNA targeting one of the three KLF1 binding motifs of BCL11A locates in +55 kb DHS of BCL11A erythroid enhancer region.

FIG. 19: Editing efficiencies induced by tBE with the pairs of sgRNA-KLF1-1-2 and its hsgRNAs targeting one of the three KLF1 binding motifs of BCL11A locates in +55 kb DHS of BCL11A erythroid enhancer region. FIG. 19A: Schematic diagram illustrating the co-transfection of the plasmids for expressing tBE system. One plasmid expresses SpD10A nickase and another expresses sgRNA-KLF1-1-2, hsgRNA-KLF1 and cytidine deaminase complex of tBE. FIG. 19B: Sanger sequencing results show the base editing efficiencies induced by tBE with indicated pairs of sgRNA/hsgRNA targeting one of the three KLF1 binding motifs of BCL11A locates in +55 kb DHS of BCL11A erythroid enhancer region.

FIG. 20: Editing efficiencies induced by tBE with the pairs of sgRNA-KLF1-1-3 and its hsgRNAs targeting one of the three KLF1 binding motifs of BCL11A locates in +55 kb DHS of BCL11A erythroid enhancer region. FIG. 20A: Schematic diagram illustrating the co-transfection of the plasmids for expressing tBE system. One plasmid expresses SpD10A nickase and another expresses sgRNA-KLF1-1-3, hsgRNA-KLF1 and cytidine deaminase complex of tBE. FIG. 20B: Sanger sequencing results show the base editing efficiencies induced by tBE with indicated pairs of sgRNA/hsgRNA targeting one of the three KLF1 binding motifs of BCL11A locates in +55 kb DHS of BCL11A erythroid enhancer region.

FIG. 21: Editing efficiencies induced by tBE with the pairs of sgRNA-KLF1-2-1 and its hsgRNAs targeting one of the three KLF1 binding motifs of BCL11A locates in +55 kb DHS of BCL11A erythroid enhancer region. FIG. 21A: Schematic diagram illustrating the co-transfection of the plasmids for expressing tBE system. One plasmid expresses SpD10A nickase and another expresses sgRNA-KLF1-2-1, hsgRNA-KLF1 and cytidine deaminase complex of tBE. FIG. 21B: Sanger sequencing results show the base editing efficiencies induced by tBE with indicated pairs of sgRNA/hsgRNA targeting one of the three KLF1 binding motifs of BCL11A locates in +55 kb DHS of BCL11A erythroid enhancer region.

FIG. 22: Editing efficiencies induced by tBE with the pairs of sgRNA-KLF1-2-2 and its hsgRNAs targeting one of the three KLF1 binding motifs of BCL11A locates in +55 kb DHS of BCL11A erythroid enhancer region. FIG. 22A: Schematic diagram illustrating the co-transfection of the plasmids for expressing tBE system. One plasmid expresses SpD10A nickase and another expresses sgRNA-KLF1-2-2, hsgRNA-KLF1 and cytidine deaminase complex of tBE. FIG. 22B: Sanger sequencing results show the base editing efficiencies induced by tBE with indicated pairs of sgRNA/hsgRNA targeting one of the three KLF1 binding motifs of BCL11A locate in +55 kb DHS of BCL11A erythroid enhancer region.

FIG. 23: Editing efficiencies induced by tBE with the pairs of sgRNA-KLF1-2-3 and its hsgRNAs targeting one of the three KLF1 binding motifs of BCL11A locates in +55 kb DHS of BCL11A erythroid enhancer region. FIG. 23A: Schematic diagram illustrating the co-transfection of the plasmids for expressing tBE system. One plasmid expresses SpD10A nickase and another expresses sgRNA-KLF1-2-3, hsgRNA-KLF1 and cytidine deaminase complex of tBE. FIG. 23B: Sanger sequencing results show the base editing efficiencies induced by tBE with indicated pairs of sgRNA/hsgRNA targeting one of the three KLF1 binding motifs of BCL11A locates in +55 kb DHS of BCL11A erythroid enhancer region.

FIG. 24: Editing efficiencies induced by tBE with the pairs of sgRNA-KLF1-2-4 and its hsgRNAs targeting one of the three KLF1 binding motifs of BCL11A locates in +55 kb DHS of BCL11A erythroid enhancer region. FIG. 24A: Schematic diagram illustrating the co-transfection of the plasmids for expressing tBE system. One plasmid expresses SpD10A nickase and another expresses sgRNA-KLF1-2-4, hsgRNA-KLF1 and cytidine deaminase complex of tBE. FIG. 24B: Sanger sequencing results show the base editing efficiencies induced by tBE with indicated pairs of sgRNA/hsgRNA targeting one of the three KLF1 binding motifs of BCL11A locates in +55 kb DHS of BCL11A erythroid enhancer region.

FIG. 25: Editing efficiencies induced by tBE with the pairs of sgRNA-KLF1-3-1 and its hsgRNAs targeting one of the three KLF1 binding motifs of BCL11A locates in 1 Mb upstream of BCL11A. FIG. 25A: Schematic diagram illustrating the co-transfection of the plasmids for expressing tBE system. One plasmid expresses SpD10A nickase and another expresses sgRNA-KLF1-3-1, hsgRNA-KLF1 and cytidine deaminase complex of tBE. FIG. 25B: Sanger sequencing results show the base editing efficiencies induced by tBE with indicated pairs of sgRNA/hsgRNA targeting one of the three KLF1 binding motifs of BCL11A locates in 1 Mb upstream of BCL11A.

FIG. 26: Editing efficiencies induced by tBE with the pairs of sgRNA-KLF1-3-2 and its hsgRNAs targeting one of the three KLF1 binding motifs of BCL11A locates in 1 Mb upstream of BCL11A. FIG. 26A: Schematic diagram illustrating the co-transfection of the plasmids for expressing tBE system. One plasmid expresses SpD10A nickase and another expresses sgRNA-KLF1-3-2, hsgRNA-KLF1 and cytidine deaminase complex of tBE. FIG. 26B: Sanger sequencing results show the base editing efficiencies induced by tBE with indicated pairs of sgRNA/hsgRNA targeting one of the three KLF1 binding motifs of BCL11A locates in 1 Mb upstream of BCL11A.

FIG. 27: Editing efficiencies induced by tBE with the pairs of sgRNA-GATA1-1 and its hsgRNAs targeting the GATA1-binding motif located in intron 4 of the NFIX gene. FIG. 27A: Schematic diagram illustrating the co-transfection of the plasmids for expressing tBE system. One plasmid expresses SpD10A nickase and another expresses sgRNA-GATA1-1, hsgRNA-GATA1 and cytidine deaminase complex of tBE. FIG. 27B: Sanger sequencing results show the base editing efficiencies induced by tBE with indicated pairs of sgRNA/hsgRNA targeting GATA1-binding motif located in intron 4 of the NFIX gene.

FIG. 28: Editing efficiencies induced by tBE with the pairs of sgRNA-GATA1-2 and its hsgRNAs targeting the GATA1-binding motif located in intron 4 of the NFIX gene. FIG. 28A: Schematic diagram illustrating the co-transfection of the plasmids for expressing tBE system. One plasmid expresses SpD10A nickase and another expresses sgRNA-GATA1-2, hsgRNA-GATA1 and cytidine deaminase complex of tBE. FIG. 28B: Sanger sequencing results show the base editing efficiencies induced by tBE with indicated pairs of sgRNA/hsgRNA targeting GATA1-binding motif located in intron 4 of the NFIX gene.

FIG. 29: Editing efficiencies induced by tBE with the pairs of sgRNA-ZBTB7A-1-1 and its hsgRNAs targeting one of the two ZBTB7A-binding motifs located in HBG1 enhancer. FIG. 29A: Schematic diagram illustrating the co-transfection of the plasmids for expressing tBE system. One plasmid expresses SpD10A nickase and another expresses sgRNA-ZBTB7A-1-1, hsgRNA-ZBTB7A and cytidine deaminase complex of tBE. FIG. 29B: Sanger sequencing results show the base editing efficiencies induced by tBE with indicated pairs of sgRNA/hsgRNA targeting the ZBTB7A-binding motif located in HBG1 enhancer.

FIG. 30: Editing efficiencies induced by tBE with the pairs of sgRNA-ZBTB7A-1-2 and its hsgRNAs targeting one of the two ZBTB7A-binding motifs located in HBG1 enhancer. FIG. 30A: Schematic diagram illustrating the co-transfection of the plasmids for expressing tBE system. One plasmid expresses SpD10A nickase and another expresses sgRNA-ZBTB7A-1-2, hsgRNA-ZBTB7A and cytidine deaminase complex of tBE. FIG. 30B: Sanger sequencing results show the base editing efficiencies induced by tBE with indicated pairs of sgRNA/hsgRNA targeting the ZBTB7A-binding motif located in HBG1 enhancer.

FIG. 31: Editing efficiencies induced by tBE with the pairs of sgRNA-ZBTB7A-1-3 and its hsgRNAs targeting one of the two ZBTB7A-binding motifs located in HBG1 enhancer. FIG. 31A: Schematic diagram illustrating the co-transfection of the plasmids for expressing tBE system. One plasmid expresses SpD10A nickase and another expresses sgRNA-ZBTB7A-1-3, hsgRNA-ZBTB7A and cytidine deaminase complex of tBE. FIG. 31B: Sanger sequencing results show the base editing efficiencies induced by tBE with indicated pairs of sgRNA/hsgRNA targeting the ZBTB7A-binding motif located in HBG1 enhancer.

FIG. 32: Editing efficiencies induced by tBE with the pairs of sgRNA-ZBTB7A-1-5 and its hsgRNAs targeting one of the two ZBTB7A-binding motifs located in HBG1 enhancer. FIG. 32A: Schematic diagram illustrating the co-transfection of the plasmids for expressing tBE system. One plasmid expresses SpD10A nickase and another expresses sgRNA-ZBTB7A-1-5, hsgRNA-ZBTB7A and cytidine deaminase complex of tBE. FIG. 32B: Sanger sequencing results show the base editing efficiencies induced by tBE with indicated pairs of sgRNA/hsgRNA targeting the ZBTB7A-binding motif located in HBG1 enhancer.

FIG. 33: Editing efficiencies induced by tBE with the pairs of sgRNA-ZBTB7A-2 and its hsgRNAs targeting another one of the two ZBTB7A-binding motifs located in HBG1/2 promoter. FIG. 33A: Schematic diagram illustrating the co-transfection of the plasmids for expressing tBE system. One plasmid expresses SpD10A nickase and another expresses sgRNA-ZBTB7A-2, hsgRNA-ZBTB7A and cytidine deaminase complex of tBE. FIG. 33B: Sanger sequencing results show the base editing efficiencies induced by tBE with indicated pairs of sgRNA/hsgRNA targeting another one of the two ZBTB7A-binding motifs located in HBG1/2 promoter.

FIG. 34: Identification of new cytidine deaminase inhibitors. FIG. 34a, Schematic diagrams illustrate the APOBEC family members that have single- or dual-CDA domains (left) and BEs that were constructed with one or two CDA domains of dual-domain APOBECs (right). FIG. 34b, Editing frequencies induced by the indicated BEs at one representative genomic locus. FIG. 34c, Statistical analysis of normalized editing frequencies, setting the ones induced by the single-CDA-containing BEs as 100%. n=78 (hA3BCDA2-nSpCas9-BE, hA3B-BE3, mA3CDA1-nSpCas9-BE and mA3-BE3) or 74 (hA3FCDA2-nSpCas9-BE and hA3F-BE3) edited cytosines at seven on-target sites from three independent experiments shown in FIG. 34b. FIG. 34d, Schematic diagrams illustrate the fusion of different dCDI domains to the N-terminus of mA3CDA1-nSpCas9-BE (mA3CDA1-BE3). e, Editing frequencies induced by the indicated BEs at one representative genomic locus. f, Statistical analysis of normalized editing frequencies, setting the ones induced by the BEs without dCDI domain as 100%. n=57 edited cytosines at five on-target sites from three independent experiments shown in FIG. 34e. FIG. 34b,e NT, non-transfected control. Data are presented as mean±s.d. from three independent experiments. FIG. 34c,f P value, two-tailed Student's t test. The median and interquartile range (IQR) are shown.

FIG. 35: Characterization of new cytidine deaminase inhibitors. FIG. 35a, Schematic diagrams illustrate base editors constructed by fusing the indicated CDA domains to nSpCas9 and uracil DNA glycosylase inhibitor (UGI). The regulatory CDA domains are in grey shadow and the active CDA domains are in colors. NLS, nuclear localization sequence; XTEN and SGGS, linker peptides. FIG. 35b, C-to-T editing frequencies induced by the indicated BEs at six genomic loci. FIG. 35c, Schematic diagrams illustrate the fusion of different dCDI domains to the N-terminus of BE3 and hA3A-BE3. FIG. 35d, C-to-T editing frequencies induced by the indicated BEs at four genomic loci. FIG. 35b,d Data are presented as mean±s.d. from three independent experiments.

FIG. 36: Editing efficiencies induced by tBE with the pairs of sgRNA-T43I-1˜2, sgRNA-C747Y-G748K/R/E, sgRNA-S755N and theirs hsgRNAs targeting the coding sequences of Znf4 in BCL11A. FIG. 36A: Schematic diagram illustrating the co-transfection of the plasmids for expressing tBE system. One plasmid expresses SpD10A nickase and another expresses sgRNA-T43I-1˜2, sgRNA-C747Y-G748K/R/E, sgRNA-S755N, different hsgRNA-T43I-1-1˜3, hsgRNA-T43I-2-1˜3, hsgRNA-C747Y-G748K/R/E-1˜3, hsgRNA-S755N-1˜3 and cytidine deaminase complex of tBE. FIG. 36B: Sanger sequencing results show the base editing efficiencies induced by tBE with indicated pairs of sgRNA/hsgRNA targeting the coding sequences of Znf4 in BCL11A.

FIG. 37: Editing efficiencies induced by tBE with the pairs of sgRNA-L757F-1˜2, sgRNA-L757F-T758I, sgRNA-V759I and theirs hsgRNAs targeting the coding sequences of Znf4 in BCL11A. FIG. 37A: Schematic diagram illustrating the co-transfection of the plasmids for expressing tBE system. One plasmid expresses SpD10A nickase and another expresses sgRNA-L757F-1˜2, sgRNA-L757F-T758I, sgRNA-V759I, different hsgRNA-L757F-1-1˜3, hsgRNA-L757F-2-1˜3, hsgRNA-L757F-T758I-1˜3, hsgRNA-V759I-1˜2 and cytidine deaminase complex of tBE. FIG. 37B: Sanger sequencing results show the base editing efficiencies induced by tBE with indicated pairs of sgRNA/hsgRNA targeting the coding sequences of Znf4 in BCL11A.

FIG. 38: Editing efficiencies induced by tBE with the pairs of sgRNA-H760Y, sgRNA-R761K, sgRNA-R761K-R762K, sgRNA-R762K-S763N and theirs hsgRNAs targeting the coding sequences of Znf4 in BCL11A. FIG. 38A: Schematic diagram illustrating the co-transfection of the plasmids for expressing tBE system. One plasmid expresses SpD10A nickase and another expresses sgRNA-H760Y, sgRNA-R761K, sgRNA-R761K-R762K, sgRNA-R762K-S763N, different hsgRNA-H760Y-1˜2, hsgRNA-R761K-1˜2, hsgRNA-R761K-R762K-1˜3, hsgRNA-R762K-S763N-1˜3 and cytidine deaminase complex of tBE. FIG. 38B: Sanger sequencing results show the base editing efficiencies induced by tBE with indicated pairs of sgRNA/hsgRNA targeting the coding sequences of Znf4 in BCL11A.

FIG. 39: Editing efficiencies induced by tBE with the pairs of sgRNA-H764Y and its hsgRNAs targeting the coding sequences of Znf4 in BCL11A, the pairs of sgRNA-G766N/S/D, sgRNA-G766N/S/D-E767K, sgRNA-R768K and theirs hsgRNAs targeting the coding sequences of the linker between BCL11A's Znf4 and Znf5. FIG. 39A: Schematic diagram illustrating the co-transfection of the plasmids for expressing tBE system. One plasmid expresses SpD10A nickase and another expresses sgRNA-H764Y, sgRNA-G766N/S/D, sgRNA-G766N/S/D-E767K, sgRNA-R768K, different hsgRNA-H764Y-1˜2, hsgRNA-G766N/S/D-1˜3, hsgRNA-G766N/S/D-E767K-1˜2, hsgRNA-R768K-1˜3 and cytidine deaminase complex of tBE. FIG. 39B: Sanger sequencing results show the base editing efficiencies induced by tBE with indicated pairs of sgRNA/hsgRNA targeting the coding sequences of Znf4 in BCL11A and the coding sequences of the linker between BCL11A's Znf4 and Znf5.

FIG. 40: Editing efficiencies induced by tBE with the pairs of sgRNA-P769F/S/L and its hsgRNAs targeting the coding sequences of the linker between BCL11A's Znf4 and Znf5, the pairs of sgRNA-C775Y, sgRNA-A778V, sgRNA-A778T and theirs hsgRNAs targeting the coding sequences of Znf5 in BCL11A. FIG. 40A: Schematic diagram illustrating the co-transfection of the plasmids for expressing tBE system. One plasmid expresses SpD10A nickase and another expresses sgRNA-P769F/S/L, sgRNA-C775Y, sgRNA-A778V, sgRNA-A778T, different hsgRNA-P769F/S/L-1˜-3, hsgRNA-C775Y-1˜3, hsgRNA-A778V-1˜3, hsgRNA-A778T-1˜3 and cytidine deaminase complex of tBE. FIG. 40B: Sanger sequencing results show the base editing efficiencies induced by tBE with indicated pairs of sgRNA/hsgRNA targeting the coding sequences of the linker between BCL11A's Znf4 and Znf5 and the coding sequences of Znf5 in BCL11A.

FIG. 41: Editing efficiencies induced by tBE with the pairs of sgRNA-A778V-A780V, sgRNA-C779Y-A780T, sgRNA-Q781*, sgRNA-S782N and theirs hsgRNAs targeting the coding sequences of Znf5 in BCL11A. FIG. 41A: Schematic diagram illustrating the co-transfection of the plasmids for expressing tBE system. One plasmid expresses SpD10A nickase and another expresses sgRNA-A778V-A780V, sgRNA-C779Y-A780T, sgRNA-Q781*, sgRNA-S782N, different hsgRNA-A778V-A780V-1˜2, hsgRNA-C779Y-A780T-1˜3, hsgRNA-Q781*-1˜3, hsgRNA-S782N-1˜3 and cytidine deaminase complex of tBE. FIG. 41B: Sanger sequencing results show the base editing efficiencies induced by tBE with indicated pairs of sgRNA/hsgRNA targeting the coding sequences of Znf5 in BCL11A.

FIG. 42: Editing efficiencies induced by tBE with the pairs of sgRNA-S783N, sgRNA-L785F, sgRNA-L785F-T786I, sgRNA-R787K and theirs hsgRNAs targeting the coding sequences of Znf5 in BCL11A. FIG. 42A: Schematic diagram illustrating the co-transfection of the plasmids for expressing tBE system. One plasmid expresses SpD10A nickase and another expresses sgRNA-S783N, sgRNA-L785F, sgRNA-L785F-T786I, sgRNA-R787K, different hsgRNA-S783N-1, sgRNA-L785F-1˜3, sgRNA-L785F-T786I-1˜3, sgRNA-R787K-1 and cytidine deaminase complex of tBE. FIG. 42B: Sanger sequencing results show the base editing efficiencies induced by tBE with indicated pairs of sgRNA/hsgRNA targeting the coding sequences of Znf5 in BCL11A.

FIG. 43: Editing efficiencies induced by tBE with the pairs of sgRNA-T791M-H792Y-1, sgRNA-T791M-H792Y-2, sgRNA-H792Y and theirs hsgRNAs targeting the coding sequences of Znf5 in BCL11A, the pairs of sgRNA-Q794* and its hsgRNAs targeting the coding sequences of the linker between BCL11A's Znf5 and Znf6 FIG. 43A: Schematic diagram illustrating the co-transfection of the plasmids for expressing tBE system. One plasmid expresses SpD10A nickase and another expresses sgRNA-T791M-H792Y-1˜2, sgRNA-H792Y, sgRNA-Q794*, different hsgRNA-T791M-H792Y-1-1˜3, hsgRNA-T791M-H792Y-2-1˜3, hsgRNA-H792Y-1˜3, hsgRNA-Q794*-1˜3 and cytidine deaminase complex of tBE. FIG. 43B: Sanger sequencing results show the base editing efficiencies induced by tBE with indicated pairs of sgRNA/hsgRNA targeting the coding sequences of Znf5 in BCL11A and the coding sequences of the linker between BCL11A's Znf5 and Znf6.

FIG. 44: Editing efficiencies induced by tBE with the pairs of sgRNA-G796K/R/E, sgRNA-G796K/R/E-D798N and theirs hsgRNAs targeting the coding sequences of the linker between BCL11A's Znf5 and Znf6, the pairs of sgRNA-P808F/S/L, sgRNA-S813N and theirs hsgRNAs targeting the coding sequences of Znf6 in BCL11A. FIG. 44A: Schematic diagram illustrating the co-transfection of the plasmids for expressing tBE system. One plasmid expresses SpD10A nickase and another expresses sgRNA-T791M-H792Y-1˜2, sgRNA-H792Y, sgRNA-Q794*, different hsgRNA-T791M-H792Y-1-1˜3, hsgRNA-T791M-H792Y-2-1˜3, hsgRNA-H792Y-1˜3, hsgRNA-Q794*-1˜3 and cytidine deaminase complex of tBE. FIG. 44B: Sanger sequencing results show the base editing efficiencies induced by tBE with indicated pairs of sgRNA/hsgRNA targeting the coding sequences of the linker between BCL11A's Znf5 and Znf6 and the coding sequences of Znf6 in BCL11A.

FIG. 45: Editing efficiencies induced by tBE with the pairs of sgRNA-S813N-2, sgRNA-E816K and theirs hsgRNAs targeting the coding sequences of Znf6 in BCL11A, the pairs of sgRNA-R826* and its hsgRNA targeting the coding sequences of the loop behind Znf6 in BCL11A. FIG. 45A: Schematic diagram illustrating the co-transfection of the plasmids for expressing tBE system. One plasmid expresses SpD10A nickase and another expresses sgRNA-S813N-2, sgRNA-E816K, sgRNA-R826*, different hsgRNA-S813N-2-1˜2, hsgRNA-E816K-1, hsgRNA-R826*-1˜3 and cytidine deaminase complex of tBE. FIG. 45B: Sanger sequencing results show the base editing efficiencies induced by tBE with indicated pairs of sgRNA/hsgRNA targeting the coding sequences of Znf6 in BCL11A and the coding sequences of the loop behind Znf6 in BCL11A.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

### Base Editors for Promoting Expression of Gamma-Globin

One embodiment of the present disclosure provides a newly designed base editor, referred to as transformer Base Editor (tBE), which can specifically edit cytosines in target regions with no observable off-target mutations. In the tBE system, a cytidine deaminase is fused with a nucleobase deaminase inhibitor to inhibit the activity of the nucleobase deaminase until the tBE complex is assembled at the target genomic site. In some embodiments, the tBE employs a sgRNA to bind at the target genomic site and a helper sgRNA (hsgRNA) to bind at a nearby region upstream to the target genomic site. The binding of two sgRNAs can guide the components of tBE to correctly assemble at the target genomic site for efficient base editing. Upon such assembly, a protease in the tBE system is activated, capable of cleaving the nucleobase deaminase inhibitor off from the nucleobase deaminase, which becomes activated.

The experimental example further tested a listing of designed sgRNA/hsgRNA sequences that target certain elements at the γ-globin promoter and/or other proteins whose expression impacts the expression of the γ-globin gene. For instance, the expression of the γ-globin is increased when the expression of BCL11A erythroid enhancer is impaired by a targeted mutation. Alternatively, when the BCL11A binding motif at the γ-globin promoter is mutated, the expression of the γ-globin gene can also be increased. Interestingly, the tBE technology can simultaneously target both the BCL11A's CREs and the BCL11A binding motif at γ-globin promoter, which is contemplated to achieve even higher efficiency in activating γ-globin gene expression.

Moreover, sgRNA/hsgRNA sequences have also been designed and tested that target other protein factors that can influence the expression of γ-globin. For instance, KLF1 is an erythroid transcription factor that activates BCL11A expression directly by binding BCL11A's promoter; another protein, NFIX, regulates the expression of KLF1; yet, ZBTB7A (zinc finger and BTB domain containing 7A) binds a γ-globin promoter and represses its expression. Targeted genomic editing that disrupts the expression of any of these protein factors can lead to activation of the γ-globin, useful for treating diseases such as beta-thalassemia and sickle cell anemia. The data demonstrate that these designed sgRNA/hsgRNA sequences led to excellent editing efficiency and specificity.

In accordance with one embodiment of the present disclosure, therefore, provided is a base editing system, or one or more polynucleotides encoding the base editing system, useful for increasing the expression of the γ-globin gene in a target cell.

In some embodiments, the base editing system includes a CRISPR-associated (Cas) protein, a nucleobase deaminase, a single-guide RNA (sgRNA)/helper single-guide RNA (hsgRNA) pair targeting the BCL11A erythroid enhancer and/or the γ-globin promoter.

“Guide RNAs” are non-coding short RNA sequences which bind to the complementary target DNA sequences. A guide RNA first binds to the Cas enzyme and the gRNA sequence guides the complex via pairing to a specific location on the DNA, where Cas performs its endonuclease activity by cutting the target DNA strand. A “single guide RNA” frequently simply referred to as “guide RNA”, refers to synthetic or expressed single guide RNA (sgRNA) that consists of both the crRNA and tracrRNA as a single construct. The tracrRNA portion is responsible for Cas endonuclease activity and the crRNA portion binds to the target specific DNA region. Therefore, the trans activating RNA (tracrRNA, or scaffold region) and crRNA are two key components and are joined by tetraloop which results in formation of sgRNA. Guide RNA targets the complementary sequences by simple Watson-Crick base pairing. TracrRNA are base pairs having a stemloop structure in itself and attaches to the endonuclease enzyme. crRNA includes a spacer, complementary to the target sequence, flanked region due to repeat sequences.

Example spacer sequences for the sgRNA/hsgRNA pair targeting the BCL11A erythroid enhancer are provided in Tables 1-2. In some embodiments, the sgRNA includes the nucleic acid sequence of any one of SEQ ID NO:1-10, the hsgRNA includes the nucleic acid sequence of any one of SEQ ID NO:11-28. The sgRNA may any one of SEQ ID NO:2, 4, 6, 8, or 10, which is 20 nt in length. In some embodiments, the sgRNA includes at least a 10 nt fragment of any of these sequences, such as SEQ ID NO:1, 3, 5, 7, or 9. Such as apparent in these examples, the 10 nt fragment is preferably proximate to the PAM site. Such preference applies here as well in other examples as shown herein. The hsgRNA may include a spacer (complementary region) that is about 10 nt in length (e.g., SEQ ID NO:11, 13, 15, 17, 19, 21, 23, 25, and 27), or 20 nt in length (e.g., SEQ ID NO:12, 14, 16, 18,20, 22, 24, 26 and 28). In some embodiments, the sgRNA includes the nucleic acid sequence of SEQ ID NO:1, and the hsgRNA comprises the nucleic acid of SEQ ID NO:17. In some embodiments, the sgRNA includes the nucleic acid sequence of SEQ ID NO:1, and the hsgRNA comprises the nucleic acid of SEQ ID NO:18. In some embodiments, the sgRNA includes the nucleic acid sequence of SEQ ID NO:4, and the hsgRNA comprises the nucleic acid of SEQ ID NO:11. In some embodiments, the sgRNA includes the nucleic acid sequence of SEQ ID NO:4, and the hsgRNA comprises the nucleic acid of SEQ ID NO:12.

Example spacer sequences for the sgRNA/hsgRNA pair targeting the γ-globin promoter (e.g., the BCL11A binding motif) are provided in Tables 3-4. In some embodiments, the sgRNA includes the nucleic acid sequence of SEQ ID NO:29-30, the hsgRNA includes the nucleic acid sequence of any one of SEQ ID NO:31-36. The hsgRNA may include a spacer (complementary region) that is about 10 nt in length (e.g., SEQ ID NO:31, 33 and 35), or 20 nt in length (e.g., SEQ ID NO:32, 34 and 36). In some embodiments, the sgRNA includes the nucleic acid sequence of SEQ ID NO:29-30, and the hsgRNA comprises the nucleic acid of SEQ ID NO:33. In some embodiments, the sgRNA includes the nucleic acid sequence of SEQ ID NO:29-30, and the hsgRNA comprises the nucleic acid of SEQ ID NO:34.

Example spacer sequences for the sgRNA/hsgRNA pair targeting one of the KLF1 motifs in BCL11A's CREs are provided in Tables 5. In some embodiments, the sgRNA includes the nucleic acid sequence of any one of SEQ ID NO:37-42, the hsgRNA belonging to the same sub Table with its sgRNA includes the nucleic acid sequence of any one of SEQ ID NO:55-62. The sgRNA may include a spacer (complementary region) that is about 10 nt in length (e.g., SEQ ID NO:37, 39 and 41), or 20 nt in length (e.g., SEQ ID NO:38, 40 and 42). The hsgRNA may include a spacer (complementary region) that is about 10 nt in length (e.g., SEQ ID NO:55, 57, 59, 61, 63, 65, 67, 69, 71, 73, 75, 77 and 79), or 20 nt in length (e.g., SEQ ID NO:56, 58, 60, 62, 64, 66, 68, 70, 72, 74, 76, 78 and 80).

Example spacer sequences for the sgRNA/hsgRNA pair targeting another of the KLF1 motifs in BCL11A's CREs are provided in Tables 6. In some embodiments, the sgRNA includes the nucleic acid sequence of any one of SEQ ID NO:43-50, the hsgRNA belonging to the same sub Table with its sgRNA includes the nucleic acid sequence of any one of SEQ ID NO:81-104. The sgRNA include a spacer (complementary region) that is about 10 nt in length (e.g., SEQ ID NO:43, 45, 47 and 49), or 20 nt in length (e.g., SEQ ID NO:44, 46, 48 and 50). The hsgRNA may include a spacer (complementary region) that is about 10 nt in length (e.g., SEQ ID NO:81, 83, 85, 87, 89, 91, 93, 95, 97, 99, 101 and 103), or 20 nt in length (e.g., SEQ ID NO:82, 84, 86, 88, 90, 92, 94, 96, 98, 100, 102 and 104).

Example spacer sequences for the sgRNA/hsgRNA pair targeting yet another of the KLF1 motifs in BCL11A's CREs are provided in Table 7. In some embodiments, the sgRNA includes the nucleic acid sequence of any one of SEQ ID NO:51-54, the hsgRNA belonging to the same sub Table with its sgRNA includes the nucleic acid sequence of any one of SEQ ID NO:105-116. The sgRNA include a spacer (complementary region) that is about 10 nt in length (e.g., SEQ ID NO:51 and 53), or 20 nt in length (e.g., SEQ ID NO:52 and 54). The hsgRNA may include a spacer (complementary region) that is about 10 nt in length (e.g., SEQ ID NO:105, 107, 109, 111, 113 and 115), or 20 nt in length (e.g., SEQ ID NO:106, 108, 110, 112, 114 and 116).

Example spacer sequences for the sgRNA/hsgRNA pair targeting a GATA1-binding motif of NFIX (Nuclear Factor IX) CRE are provided in Table 8. In some embodiments, the sgRNA includes the nucleic acid sequence of any one of SEQ ID NO:117-122, the hsgRNA belonging to the same sub Table with its sgRNA includes the nucleic acid sequence of any one of SEQ ID NO:123-138. The sgRNA include a spacer (complementary region) that is about 10 nt in length (e.g., SEQ ID NO:117, 119 and 121), or 20 nt in length (e.g., SEQ ID NO:118, 120 and 122). The hsgRNA may include a spacer (complementary region) that is about 10 nt in length (e.g., SEQ ID NO:123, 125, 127, 129, 131, 133, 135 and 137), or 20 nt in length (e.g., SEQ ID NO: 124, 126, 128, 130, 132, 134, 136 and 138).

Example spacer sequences for the sgRNA/hsgRNA pair targeting a ZBTB7A-binding motif of HBG1/2's CRE are provided in Tables 9 and 10. In some embodiments, the sgRNA includes the nucleic acid sequence of any one of SEQ ID NO:139-150, the hsgRNA belonging to the same sub Table with its sgRNA includes the nucleic acid sequence of any one of SEQ ID NO:151-190. The sgRNA include a spacer (complementary region) that is about 10 nt in length (e.g., SEQ ID NO:139, 141, 143, 145, 147 and 149), or 20 nt in length (e.g., SEQ ID NO:140, 142, 144, 146, 148 and 150). The hsgRNA may include a spacer (complementary region) that is about 10 nt in length (e.g., SEQ ID NO:151, 153, 155, 157, 159, 161, 163, 165, 167, 169, 171, 173, 175, 177, 179, 181, 183, 185, 187 and 189), or 20 nt in length (e.g., SEQ ID NO:152, 154, 156, 158 160, 162, 164, 166, 168, 170, 172, 174, 176, 178, 180, 182, 184, 186, 188 and 190).

Additional example spacer sequences for the sgRNA/hsgRNA pair targeting various other sites are provided in Table 11. Such example sites include, e.g., T743, T743, C747 and G748, S755, L757, L757, L757 and T758, V759, H760, R761, R761 and R762, R761 and S763, H764, G766, G766 and E767, R768, P769, C775, A778, A778, A778 and A780, C779 and A780, Q781, S782, S783, L785, L785 and T786, S783, T791 and H792, T791 and H792, H792, Q794, G796, G796 and D798, P808, S813, S813, E816, or R826 of BCL11A. In some embodiments, the sgRNA includes a nucleic acid sequence selected from the group consisting of SEQ ID NO:353-430, and the hsgRNA includes a nucleic acid sequence selected from the group consisting of SEQ ID NO:431-628.

In some embodiments, the sgRNA includes a nucleic acid sequence selected from the group consisting of SEQ ID NO:1-10, and the hsgRNA includes a nucleic acid sequence selected from the group consisting of SEQ ID NO:11-28.

In some embodiments, the sgRNA includes a nucleic acid sequence selected from the group consisting of SEQ ID NO:29-30, and the hsgRNA includes a nucleic acid sequence selected from the group consisting of SEQ ID NO:31-36.

In some embodiments, the sgRNA includes a nucleic acid sequence selected from the group consisting of SEQ ID NO:37-38, and the hsgRNA includes a nucleic acid sequence selected from the group consisting of SEQ ID NO:55-62. In some embodiments, the sgRNA includes a nucleic acid sequence selected from the group consisting of SEQ ID NO:39-40, and the hsgRNA includes a nucleic acid sequence selected from the group consisting of SEQ ID NO:63-70. In some embodiments, the sgRNA includes a nucleic acid sequence selected from the group consisting of SEQ ID NO:41-42, and the hsgRNA includes a nucleic acid sequence selected from the group consisting of SEQ ID NO:71-80.

In some embodiments, the sgRNA includes a nucleic acid sequence selected from the group consisting of SEQ ID NO:43-44, and the hsgRNA includes a nucleic acid sequence selected from the group consisting of SEQ ID NO:81-104. In some embodiments, the sgRNA includes a nucleic acid sequence selected from the group consisting of SEQ ID NO:45-46, and the hsgRNA includes a nucleic acid sequence selected from the group consisting of SEQ ID NO:81-104. In some embodiments, the sgRNA includes a nucleic acid sequence selected from the group consisting of SEQ ID NO:47-48, and the hsgRNA includes a nucleic acid sequence selected from the group consisting of SEQ ID NO:81-104. In some embodiments, the sgRNA includes a nucleic acid sequence selected from the group consisting of SEQ ID NO:49-50, and the hsgRNA includes a nucleic acid sequence selected from the group consisting of SEQ ID NO:87-98.

In some embodiments, the sgRNA includes a nucleic acid sequence selected from the group consisting of SEQ ID NO:51-52, and the hsgRNA includes a nucleic acid sequence selected from the group consisting of SEQ ID NO:105-116. In some embodiments, the sgRNA includes a nucleic acid sequence selected from the group consisting of SEQ ID NO:53-54, and the hsgRNA includes a nucleic acid sequence selected from the group consisting of SEQ ID NO:105-116.

In some embodiments, the sgRNA includes a nucleic acid sequence selected from the group consisting of SEQ ID NO: 117-118, and the hsgRNA includes a nucleic acid sequence selected from the group consisting of SEQ ID NO:123-138. In some embodiments, the sgRNA includes a nucleic acid sequence selected from the group consisting of SEQ ID NO:119-120, and the hsgRNA includes a nucleic acid sequence selected from the group consisting of SEQ ID NO:123-138. In some embodiments, the sgRNA includes a nucleic acid sequence selected from the group consisting of SEQ ID NO:121-122, and the hsgRNA includes a nucleic acid sequence selected from the group consisting of SEQ ID NO:123-138.

In some embodiments, the sgRNA includes a nucleic acid sequence selected from the group consisting of SEQ ID NO: 139-140, and the hsgRNA includes a nucleic acid sequence selected from the group consisting of SEQ ID NO:151-164. In some embodiments, the sgRNA includes a nucleic acid sequence selected from the group consisting of SEQ ID NO:141-142, and the hsgRNA includes a nucleic acid sequence selected from the group consisting of SEQ ID NO:151-164. In some embodiments, the sgRNA includes a nucleic acid sequence selected from the group consisting of SEQ ID NO:143-144, and the hsgRNA includes a nucleic acid sequence selected from the group consisting of SEQ ID NO:151-164. In some embodiments, the sgRNA includes a nucleic acid sequence selected from the group consisting of SEQ ID NO:145-146, and the hsgRNA includes a nucleic acid sequence selected from the group consisting of SEQ ID NO:151-164. In some embodiments, the sgRNA includes a nucleic acid sequence selected from the group consisting of SEQ ID NO:147-148, and the hsgRNA includes a nucleic acid sequence selected from the group consisting of SEQ ID NO:165-178.

In some embodiments, the sgRNA includes a nucleic acid sequence selected from the group consisting of SEQ ID NO: 149-150, and the hsgRNA includes a nucleic acid sequence selected from the group consisting of SEQ ID NO:179-190.

In some embodiments, the sgRNA includes a nucleic acid sequence selected from the group consisting of SEQ ID NO:353-354, and the hsgRNA includes a nucleic acid sequence selected from the group consisting of SEQ ID NO:431-436. In some embodiments, the sgRNA includes a nucleic acid sequence selected from the group consisting of SEQ ID NO:355-356, and the hsgRNA includes a nucleic acid sequence selected from the group consisting of SEQ ID NO:437-442. In some embodiments, the sgRNA includes a nucleic acid sequence selected from the group consisting of SEQ ID NO:357-358, and the hsgRNA includes a nucleic acid sequence selected from the group consisting of SEQ ID NO:443-448.

In some embodiments, the sgRNA includes a nucleic acid sequence selected from the group consisting of SEQ ID NO:359-360, and the hsgRNA includes a nucleic acid sequence selected from the group consisting of SEQ ID NO:449-454. In some embodiments, the sgRNA includes a nucleic acid sequence selected from the group consisting of SEQ ID NO:361-362, and the hsgRNA includes a nucleic acid sequence selected from the group consisting of SEQ ID NO:455-460. In some embodiments, the sgRNA includes a nucleic acid sequence selected from the group consisting of SEQ ID NO:363-364, and the hsgRNA includes a nucleic acid sequence selected from the group consisting of SEQ ID NO:461-466.

In some embodiments, the sgRNA includes a nucleic acid sequence selected from the group consisting of SEQ ID NO:365-366, and the hsgRNA includes a nucleic acid sequence selected from the group consisting of SEQ ID NO:467-472. In some embodiments, the sgRNA includes a nucleic acid sequence selected from the group consisting of SEQ ID NO:367-368, and the hsgRNA includes a nucleic acid sequence selected from the group consisting of SEQ ID NO:473-476. In some embodiments, the sgRNA includes a nucleic acid sequence selected from the group consisting of SEQ ID NO:369-370, and the hsgRNA includes a nucleic acid sequence selected from the group consisting of SEQ ID NO:477-480.

In some embodiments, the sgRNA includes a nucleic acid sequence selected from the group consisting of SEQ ID NO:371-372, and the hsgRNA includes a nucleic acid sequence selected from the group consisting of SEQ ID NO:481-484. In some embodiments, the sgRNA includes a nucleic acid sequence selected from the group consisting of SEQ ID NO:373-374, and the hsgRNA includes a nucleic acid sequence selected from the group consisting of SEQ ID NO:485-488. In some embodiments, the sgRNA includes a nucleic acid sequence selected from the group consisting of SEQ ID NO:375-376, and the hsgRNA includes a nucleic acid sequence selected from the group consisting of SEQ ID NO:489-492.

In some embodiments, the sgRNA includes a nucleic acid sequence selected from the group consisting of SEQ ID NO:377-378, and the hsgRNA includes a nucleic acid sequence selected from the group consisting of SEQ ID NO:493-496. In some embodiments, the sgRNA includes a nucleic acid sequence selected from the group consisting of SEQ ID NO:379-380, and the hsgRNA includes a nucleic acid sequence selected from the group consisting of SEQ ID NO:497-502. In some embodiments, the sgRNA includes a nucleic acid sequence selected from the group consisting of SEQ ID NO:381-382, and the hsgRNA includes a nucleic acid sequence selected from the group consisting of SEQ ID NO:503-506.

In some embodiments, the sgRNA includes a nucleic acid sequence selected from the group consisting of SEQ ID NO:383-384, and the hsgRNA includes a nucleic acid sequence selected from the group consisting of SEQ ID NO:507-512. In some embodiments, the sgRNA includes a nucleic acid sequence selected from the group consisting of SEQ ID NO:385-386, and the hsgRNA includes a nucleic acid sequence selected from the group consisting of SEQ ID NO:513-518. In some embodiments, the sgRNA includes a nucleic acid sequence selected from the group consisting of SEQ ID NO:387-388, and the hsgRNA includes a nucleic acid sequence selected from the group consisting of SEQ ID NO:519-524.

In some embodiments, the sgRNA includes a nucleic acid sequence selected from the group consisting of SEQ ID NO:389-390, and the hsgRNA includes a nucleic acid sequence selected from the group consisting of SEQ ID NO:525-530. In some embodiments, the sgRNA includes a nucleic acid sequence selected from the group consisting of SEQ ID NO:391-392, and the hsgRNA includes a nucleic acid sequence selected from the group consisting of SEQ ID NO:531-536. In some embodiments, the sgRNA includes a nucleic acid sequence selected from the group consisting of SEQ ID NO:393-394, and the hsgRNA includes a nucleic acid sequence selected from the group consisting of SEQ ID NO:537-540.

In some embodiments, the sgRNA includes a nucleic acid sequence selected from the group consisting of SEQ ID NO:395-396, and the hsgRNA includes a nucleic acid sequence selected from the group consisting of SEQ ID NO:541-546. In some embodiments, the sgRNA includes a nucleic acid sequence selected from the group consisting of SEQ ID NO:397-398, and the hsgRNA includes a nucleic acid sequence selected from the group consisting of SEQ ID NO:547-552. In some embodiments, the sgRNA includes a nucleic acid sequence selected from the group consisting of SEQ ID NO:399-400, and the hsgRNA includes a nucleic acid sequence selected from the group consisting of SEQ ID NO:553-558.

In some embodiments, the sgRNA includes a nucleic acid sequence selected from the group consisting of SEQ ID NO:401-402, and the hsgRNA includes a nucleic acid sequence selected from the group consisting of SEQ ID NO:559-560. In some embodiments, the sgRNA includes a nucleic acid sequence selected from the group consisting of SEQ ID NO:403-404, and the hsgRNA includes a nucleic acid sequence selected from the group consisting of SEQ ID NO:561-566. In some embodiments, the sgRNA includes a nucleic acid sequence selected from the group consisting of SEQ ID NO:405-406, and the hsgRNA includes a nucleic acid sequence selected from the group consisting of SEQ ID NO:567-572.

In some embodiments, the sgRNA includes a nucleic acid sequence selected from the group consisting of SEQ ID NO:407-408, and the hsgRNA includes a nucleic acid sequence selected from the group consisting of SEQ ID NO:573-574. In some embodiments, the sgRNA includes a nucleic acid sequence selected from the group consisting of SEQ ID NO:409-410, and the hsgRNA includes a nucleic acid sequence selected from the group consisting of SEQ ID NO:575-580. In some embodiments, the sgRNA includes a nucleic acid sequence selected from the group consisting of SEQ ID NO:411-412, and the hsgRNA includes a nucleic acid sequence selected from the group consisting of SEQ ID NO:581-586.

In some embodiments, the sgRNA includes a nucleic acid sequence selected from the group consisting of SEQ ID NO:413-414, and the hsgRNA includes a nucleic acid sequence selected from the group consisting of SEQ ID NO:587-592. In some embodiments, the sgRNA includes a nucleic acid sequence selected from the group consisting of SEQ ID NO:415-416, and the hsgRNA includes a nucleic acid sequence selected from the group consisting of SEQ ID NO:593-598. In some embodiments, the sgRNA includes a nucleic acid sequence selected from the group consisting of SEQ ID NO:415-416, and the hsgRNA includes a nucleic acid sequence selected from the group consisting of SEQ ID NO:593-598.

In some embodiments, the sgRNA includes a nucleic acid sequence selected from the group consisting of SEQ ID NO:417-418, and the hsgRNA includes a nucleic acid sequence selected from the group consisting of SEQ ID NO:599-602. In some embodiments, the sgRNA includes a nucleic acid sequence selected from the group consisting of SEQ ID NO:419-420, and the hsgRNA includes a nucleic acid sequence selected from the group consisting of SEQ ID NO:603-606. In some embodiments, the sgRNA includes a nucleic acid sequence selected from the group consisting of SEQ ID NO:421-422, and the hsgRNA includes a nucleic acid sequence selected from the group consisting of SEQ ID NO:607-612.

In some embodiments, the sgRNA includes a nucleic acid sequence selected from the group consisting of SEQ ID NO:423-424, and the hsgRNA includes a nucleic acid sequence selected from the group consisting of SEQ ID NO:613-616. In some embodiments, the sgRNA includes a nucleic acid sequence selected from the group consisting of SEQ ID NO:425-426, and the hsgRNA includes a nucleic acid sequence selected from the group consisting of SEQ ID NO:617-620. In some embodiments, the sgRNA includes a nucleic acid sequence selected from the group consisting of SEQ ID NO:427-428, and the hsgRNA includes a nucleic acid sequence selected from the group consisting of SEQ ID NO:621-622. In some embodiments, the sgRNA includes a nucleic acid sequence selected from the group consisting of SEQ ID NO:429-430, and the hsgRNA includes a nucleic acid sequence selected from the group consisting of SEQ ID NO:623-628.

In some embodiments, the base editing system targets two or more of the above target sites, e.g., BCL11A, BCL11A binding motif of γ-globin, KLF1 binding motifs of BCL11A, GATA1-binding motif of NFIX, and/or ZBTB7A-binding motif of γ-globin.

In some embodiments, the base editing system targets both the BCL11A erythroid enhancer and the γ-globin promoter. Accordingly, two pairs of sgRNA/hsgRNA are included. In a particular example, the first sgRNA/hsgRNA pair includes spacers as described in SEQ ID NO:4 and 11 (or 12), and the second sgRNA/hsgRNA pair includes spacers as described in SEQ ID NO:30 and 33 (or 34).

The term “nucleobase deaminase” as used herein, refers to a group of enzymes that catalyze the hydrolytic deamination of nucleobases such as cytidine, deoxycytidine, adenosine and deoxyadenosine. Non-limiting examples of nucleobase deaminases include cytidine deaminases and adenosine deaminases.

“Cytidine deaminase” refers to enzymes that catalyze the irreversible hydrolytic deamination of cytidine and deoxycytidine to uridine and deoxyuridine, respectively. Cytidine deaminases maintain the cellular pyrimidine pool. A family of cytidine deaminases is APOBEC (“apolipoprotein B mRNA editing enzyme, catalytic polypeptide-like”). Members of this family are C-to-U editing enzymes. Some APOBEC family members have two domains, one domain of APOBEC like proteins is the catalytic domain, while the other domain is a pseudocatalytic domain. More specifically, the catalytic domain is a zinc dependent cytidine deaminase domain and is important for cytidine deamination.

Non-limiting examples of APOBEC proteins include APOBEC1, APOBEC2, APOBEC3A, APOBEC3B, APOBEC3C, APOBEC3D, APOBEC3F, APOBEC3G, APOBEC3H, APOBEC4, and activation-induced (cytidine) deaminase (AID).

Various mutants of the APOBEC proteins are also known that have bring about different editing characteristics for base editors. For instance, for human APOBEC3A, certain mutants (e.g., W98Y, Y130F, Y132D, W104A, D131Y and P134Y) even outperform the wildtype human APOBEC3A in terms of editing efficiency or editing window. Accordingly, the term APOBEC and each of its family member also encompasses variants and mutants that have certain level (e.g., 70%, 75%, 80%, 85%, 90%, 95%, 98%, 99%) of sequence identity to the corresponding wildtype APOBEC protein or the catalytic domain and retain the cytidine deaminating activity. The variants and mutants can be derived with amino acid additions, deletions and/or substitutions. Such substitutions, in some embodiments, are conservative substitutions.

“Adenosine deaminase”, also known as adenosine aminohydrolase, or ADA, is an enzyme (EC 3.5.4.4) involved in purine metabolism. It is needed for the breakdown of adenosine from food and for the turnover of nucleic acids in tissues.

Non-limiting examples of adenosine deaminases include tRNA-specific adenosine deaminase (TadA), adenosine deaminase tRNA specific 1 (ADAT1), adenosine deaminase tRNA specific 2 (ADAT2), adenosine deaminase tRNA specific 3 (ADAT3), adenosine deaminase RNA specific B1 (ADARB1), adenosine deaminase RNA specific B2 (ADARB2), adenosine monophosphate deaminase 1 (AMPD1), adenosine monophosphate deaminase 2 (AMPD2), adenosine monophosphate deaminase 3 (AMPD3), adenosine deaminase (ADA), adenosine deaminase 2 (ADA2), adenosine deaminase like (ADAL), adenosine deaminase domain containing 1 (ADAD1), adenosine deaminase domain containing 2 (ADAD2), adenosine deaminase RNA specific (ADAR) and adenosine deaminase RNA specific B1 (ADARB1).

Some of the nucleobase deaminases have a single, catalytic domain, while others also have other domains, such as an inhibitory domain as currently discovered by the instant inventors. In some embodiments, therefore, the first fragment only includes the catalytic domain, such as mA3-CDA1, hA3F-CDA2 and hA3B-CDA2. In some embodiments, the first fragment includes at least a catalytic core of the catalytic domain.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, RanCas13b and those provided in Table A below.

TABLE A
Example Cas Proteins

| Cas protein types | Cas proteins |
|---|---|
| Cas9 | Cas9 from Staphylococcus aureus (SaCas9) |
| proteins | Cas9 from Neisseria meningitidis (NmeCas9) |
| | Cas9 from Streptococcus thermophilus (StCas9) |
| | Cas9 from Campylobacter jejuni (CjCas9) |
| Cas12a | Cas12a (Cpf1) from Acidaminococcus sp BV3L6 (AsCpf1) |
| (Cpf1) | Cas12a (Cpf1) from Francisella novicida sp BV3L6 (FnCpf1) |
| proteins | Cas12a (Cpf1) from Smithella sp SC K08D17 (SsCpf1) |
| | Cas12a (Cpf1) from Porphyromonas crevioricanis (PcCpf1) |
| | Cas12a (Cpf1) from Butyrivibrio proteoclasticus (BpCpf1) |
| | Cas12a (Cpf1) from Candidatus Methanoplasma termitum |
| | (CmtCpf1) |
| | Cas12a (Cpf1) from Leptospira inadai (LiCpf1) |
| | Cas12a (Cpf1) from Porphyromonas macacae (PmCpf1) |
| | Cas12a (Cpf1) from Peregrinibacteria bacterium |
| | GW2011 WA2 33 10 (Pb3310Cpf1) |
| | Cas12a (Cpf1) from Parcubacteria bacterium |
| | GW2011 GWC2 44 17 (Pb4417Cpf1) |
| | Cas12a (Cpf1) from Butyrivibrio sp. NC3005 (BsCpf1) |
| | Cas12a (Cpf1) from Eubacterium eligens (EeCpf1) |
| Cas12b | Cas12b (C2c1) Bacillus hisashii (BhCas12b) |
| (C2c1) | Cas12b (C2c1) Bacillus hisashii with a gain-of-function mutation |
| proteins | (see, e.g., Strecker et al., Nature Communications 10 |
| | (article 212) (2019) |
| | Cas12b (C2c1) Alicyclobacillus kakegawensis (AkCas12b) |
| | Cas12b (C2c1) Elusimicrobia bacterium (EbCas12b) |
| | Cas12b (C2c1) Laceyella sediminis (Ls) (LsCas12b) |
| Cas13 | Cas13d from Ruminococcus flavefaciens XPD3002 (RfCas13d) |
| proteins | Cas13a from Leptotrichia wadei (LwaCas13a) |
| | Cas13b from Prevotella sp. P5-125 (PspCas13b) |
| | Cas13b from Porphyromonas gulae (PguCas13b) |
| | Cas13b from Riemerella anatipestifer (RanCas13b) |
| Engineered | Nickases (mutation in one nuclease domain) |
| Cas | Catalytically inactive mutant (dCas9; mutations |
| proteins | in both of the nuclease domains) |
| | Enhanced variants with improved specificity |
| | (see, e.g., Chen et al., Nature, 550, 407-410 (2017) |

In some embodiments, the base editing system further includes a nucleobase deaminase inhibitor fused to the nucleobase deaminase. A “nucleobase deaminase inhibitor,” accordingly, refers to a protein or a protein domain that inhibits the deaminase activity of a nucleobase deaminase. In some embodiments, the second fragment includes at least an inhibitory core of the inhibitory protein/domain.

Two example nucleobase deaminase inhibitors are mA3-CDA2, hA3F-CDA1 and hA31B-CDA1 (sequences provided in Table B), which are the inhibitory domains of the corresponding nucleobase deaminases. Additional nucleobase deaminase inhibitors have been identified in the protein databases as homologues of mA3-CDA2, hA3F-CDA1 and hA3B-CDA1 (see Tables B1, B2 and B3). Their biological equivalents (e.g., having at least about 80%, 85%, 90%, 95%, 97%, 98%, 99%, 99.5% sequence identity, or having one, two, or three amino acid addition/deletion/substitution, and having nucleobase deaminase inhibitor activity) can also be prepared with known methods in the art, such as conservative amino acid substitutions.

When the nucleobase deaminase inhibitor is included, it is fused to the nucleobase deaminase but is separated by a protease cleavage site. In some embodiments, the base editing system further includes the protease that is capable of cleaving the protease cleavage site.

The protease cleavage site can be any known protease cleavage site (peptide) for any proteases. Non-limiting examples of proteases include TEV protease, TuMV protease, PPV protease, PVY protease, ZIKV protease and WNV protease. The protein sequences of example proteases and their corresponding cleavage sites are provided in Table B.

TABLE B
Example Sequences

| Name | Sequence | SEQ ID NO: |
|---|---|---|
| Mouse APOBEC3 | MSSSTLSNICLTKGLPETRFWVEGRRMDPLSEEEFYSQFYNQRVKHLCY | 191 |
| cytidine deaminase | YHRMKPYLCYQLEQFNGQAPLKGCLLSEKGKQHAEILFLDKIRSMELSQ | |
| domain 2 | VTITCYLTWSPCPNCAWQLAAFKRDRPDLILHIYTSRLYFHWKRPFQKG | |
| | LCSLWQSGILVDVMDLPQFTDCWTNFVNPKRPFWPWKGLEIISRRTQRR | |
| | LRRIKESWGLQDLVNDFGNLQLGPPMS | |
| Human | MKPHFRNTVERMYRDTFSYNFYNRPILSRRNTVWLCYEVKTKGPSRPRL | 192 |
| APOBEC3F | DAKIFRGQVYSQPEHHAEMCFLSWFCGNQLPAYKCFQITWFVSWTPCPD | |
| cytidine deaminase | CVAKLAEFLAEHPNVTLTISAARLYYYWERDYRRALCRLSQAGARVKIM | |
| domain 1 | DDEEFAYCWENFVYSEG | |
| Human | MNPQIRNPMERMYRDTFYDNFENEPILYGRSYTWLCYEVKIKRGRSNLL | 193 |
| APOBEC3B | WDTGVFRGQVYFKPQYHAEMCFLSWFCGNQLPAYKCFQITWFVSWTPCP | |
| cytidine deaminase | DCVAKLAEFLSEHPNVTLTISAARLYYYWERDYRRALCRLSQAGARVKI | |
| domain 1 | MDYEEFAYCWENFVYNEGQ | |
| TEV protease N- | MGESLFKGPRDYNPISSTICHLTNESDGHTTSLYGIGFGPFIITNKHLF | 194 |
| terminal domain | RRNNGTLLVQSLHGVFKVKNTTTLQQHLIDGRDMIIIRMPKDFPPFPQK | |
| | LKFREPQREERICLVTTNFQT | |
| TEV protease C- | MKSMSSMVSDTSCTFPSSDGIFWKHWIQTKDGQCGSPLVSTRDGFIVGI | 195 |
| terminal domain | HSASNFTNTNNYFTSVPKNFMELLINQEAQQWVSGWRLNADSVLWGGHK | |
| | VFMVKPEEPFQPVKEATQ | |
| TEV protease | ENLYFQS | 196 |
| cleavage site | | |
| TuMV protease | MASSNSMFRGLRDYNPISNNICHLTNVSDGASNSLYGVGFGPLILINRH | 197 |
| | LFERNNGELVIKSRHGEFVIKNTTQLHLLPIPDRDLLLIRLPKDVPPFP | |
| | QKLGFRQPEKGERICMVGSNFQTKSITSIVSETSTIMPVENSQFWKHWI | |
| | STKDGQCGSPMVSTKDGKILGLHSLANFQNSINYFAAFPDDFAEKYLHT | |
| | IEAHEWVKHWKYNTSAISWGSLNIQASQPSGLFKVSKLISDLDSTAVYA | |
| | Q | |
| TuMV protease | GGCSHQS | 198 |
| cleavage site | | |
| PPV protease | MASSKSLFRGLRDYNPIASSICQLNNSSGARQSEMFGLGFGGLIVINQH | 199 |
| | LFKRNDGELTIRSHHGEFVVKDTKTLKLLPCKGRDIVIIRLPKDFPPFP | |
| | RRLQFRTPTTEDRVCLIGSNFQTKSISSTMSETSATYPVDNSHFWKHWI | |
| | STKDGHCGLPIVSTRDGSILGLHSLANSTNTQNFYAAFPDNFETTYLSN | |
| | QDNDNWIKQWRYNPDEVCWGSLQLKRDIPQSPFTICKLLTDLDGEFVYT | |
| | Q | |
| PPV protease | QVVVHQSK | 200 |
| cleavage site | | |
| PVY protease | MASAKSLMRGLRDFNPIAQTVCRLKVSVEYGASEMYGFGFGAYIVANHH | 201 |
| | LFRSYNGSMEVQSMHGTFRVKNLHSLSVLPIKGRDIILIKMPKDFPVFP | |
| | QKLHFRAPTQNERICLVGTNFQEKYASSIITETSTTYNIPGSTFWKHWI | |
| | ETDNGHCGLPVVSTADGCIVGIHSLANNAHTTNYYSAFDEDFESKYLRT | |
| | NEHNEWVKSWVYNPDTVLWGPLKLKDSTPKGLFKTTKLVQDLIDHDVVV | |
| | EQ | |
| PVY protease | YDVRHQSR | 202 |
| cleavage site | | |
| ZIKV protease | MASDMYIERAGDITWEKDAEVTGNSPRLDVALDESGDFSLVEEDGPPMR | 203 |
| | EGGGGSGGGGSGALWDVPAPKEVKKGETTDGVYRVMTRRLLGSTQVGVG | |
| | VMQEGVFHTMWHVTKGAALRSGEGRLDPYWGDVKQDLVSYCGPWKLDAA | |
| | WDGLSEVQLLAVPPGERARNIQTLPGIFKTKDGDIGAVALDYPAGTSGS | |
| | PILDKCGRVIGLYGNGVVIKNGSYVSAITQGKREEETPVECFE | |
| ZIKV protease | KERKRRGA | 204 |
| cleavage site | | |
| WNV protease | MASSTDMWIERTADISWESDAEITGSSERVDVRLDDDGNFQLMNDPGAP | 205 |
| | WKGGGGSGGGGGVLWDTPSPKEYKKGDTTTGVYRIMTRGLLGSYQAGAG | |
| | VMVEGVFHTLWHTTKGAALMSGEGRLDPYWGSVKEDRLCYGGPWKLQHK | |
| | WNGQDEVQMIVVEPGKNVKNVQTKPGVFKTPEGEIGAVTLDFPTGTSGS | |
| | PIVDKNGDVIGLYGNGVIMPNGSYISAIVQGERMDEPIPAGFEPEML | |
| WNV protease | KQKKRGGK | 206 |
| cleavage site | | |
| MS2 | ACAUGAGGAUCACCCAUGU | 207 |
| sgRNA scaffold | GUUUGAGAGCUAGGCCAACAUGAGGAUCACCCAUGUCUGCAGGGCCUAG | 208 |
| with 2×MS2 | CAAGUUCAAAUAAGGCUAGUCCGUUAUCAACUUGGCCAACAUGAGGAUC | |
| | ACCCAUGUCUGCAGGGCCAAGUGGCACCGAGUCGGUGC | |
| PP7 | GGAGCAGACGAUAUGGCGUCGCUCC | 209 |
| sgRNA scaffold | GUUUGAGAGCUACCGGAGCAGACGAUAUGGCGUCGCUCCGGUAGCAAGU | 210 |
| with 2×PP7 | UCAAAUAAGGCUAGUCCGUUAUCAACUUGGAGCAGACGAUAUGGCGUCG | |
| | CUCCAAGUGGCACCGAGUCGGUGC | |
| boxB | GCCCUGAAGAAGGGC | 211 |
| sgRNA scaffold | GUUUGAGAGCUAGGGCCCUGAAGAAGGGCCCUAGCAAGUUCAAAUAAGG | 212 |
| with 2xboxB | CUAGUCCGUUAUCAACUUGGGCCCUGAAGAAGGGCCCAAGUGGCACCGA | |
| | GUCGGUGC | |
| MS2 coat protein | MASNFTQFVLVDNGGTGDVTVAPSNFANGIAEWISSNSRSQAYKVTCSV | 213 |
| (MCP) | RQSSAQNRKYTIKVEVPKGAWRSYLNMELTIPIFATNSDCELIVKAMQG | |
| | LLKDGNPIPSAIAANSGIY | |
| PP7 coat protein | MGSKTIVLSVGEATRTLTEIQSTADRQIFEEKVGPLVGRLRLTASLRQN | 214 |
| (PCP) | GAKTAYRVNLKLDQADVVDSGLPKVRYTQVWSHDVTIVANSTEASRKSL | |
| | YDLTKSLVATSQVEDLVVNLVPLGR | |
| boxB coat protein | MGNARTRRRERRAEKQAQWKAAN | 215 |
| (N22p) | | |
| UGI | TNLSDIIEKETGKQLVIQESILMLPEEVEEVIGNKPESDILVHTAYDES | 216 |
| | TDENVMLLTSDAPEYKPWALVIQDSNGENKIKML | |
| P2A | GSGATNFSLLKQAGDVEENPGP | 217 |
| T2A | GSGEGRGSLLTCGDVEENPGP | 218 |
| E2A | GSGQCTNYALLKLAGDVESNPGP | 219 |

TABLE B1
mA3CDA2 Core Sequence Related Domains

| | | SEQ |
|---|---|---|
| | | ID |
| Name | Sequence | NO: |
| Mouse APOBEC3 | SEKGKQHAEILFLDKIRSMELSQVTITCYLTWSPCPNCAW | 220 |
| cytidine deaminase | QLAAFKRDRPDLILHIYTSRLYFHWKRPFQKGLC | |
| domain 2 core | | |
| (AA282-AA355) | | |
| Mus spicilegus A3 | SEKGKQHAEILELDKIRSMELSQVTITCYLTWSPCPNCAW | 221 |
| (AA248-AA321) | QLAAFKRDRPDLIPHIYTSRLYFHWKRPFQKGLC | |
| Cricetulus | SEKGKQHAEILFLDKIRSMELSQVTITCYLTWSPCPNCAW | 222 |
| longicaudatus A3 | RLAAFKRDRPDLILHIYTSRLYFHWKRPFQKGLC | |
| (AA249-AA322) | | |
| Mus terricolor A3 | SEKGKQHAEILFLNKIRSMELSQVTITCYLTWSPCPNCAW | 223 |
| (AA248-AA321) | QLAAFKKDRPDLILHIYTSRLYFHWKRPFQKGLC | |
| Mus caroli A3 | SKKGKQHAEILFLDKIRSMELSQVTITCYLTWSPCPNCAW | 224 |
| (AA260-AA333) | QLAAFKRDHPDLILHIYTSRLYFHWKRPFQKGLC | |
| Mus pahari A3 | SKKGKQHAEILFLEKIRSMELSQMRITCYLTWSPCPNCAW | 225 |
| (AA263-AA336) | QLAAFQKDRPDLILHIYTSRLYFHWRRIFQKGLC | |
| Mus shortridgei A3 | SKKGKQHAEILFLEKIRSMELSQMRITCYLTWSPCPNCAW | 226 |
| (AA233-AA306) | QLAAFQKDRPDLILHIYTSRLYFHWRRIFQKGLC | |
| Mus setulosus A3 | SKKGKQHAEILELDKIRSMELSQVRITCYLTWSPCPNCAW | 227 |
| (AA29-AA302) | QLETFKKDRPDLILHIYTSRLYFHWKRAFQEGLC | |
| Grammomys | SKKGKPHAEILFLDKMWSMEELSQVRITCYLTWSPCPNCA | 228 |
| surdaster A3 | RQLAAFKKDHPGLILRIYTSRLYFYWRRKFQKGLC | |
| (AA270-AA344) | | |
| Rattus norvegicus A3 | KKGEQHVEILFLEKMRSMELSQVRITCYLTWSPCPNCARQ | 229 |
| (AA256-AA328) | LAAFKKDHPDLILRIYTSRLYFYWRKKFQKGLC | |
| Mastomys coucha A3 | SKKGRQHAEILFLEKVRSMQLSQVRITCYLTWSPCPNCAW | 230 |
| (AA258-AA331) | QLAAFKMDHPDLILRIYASRLYFHWRRAFQKGLC | |
| Cricetulus griseus | NKKGKHAEILFIDEMRSLELGQVQITCYLTWSPCPNCAQE | 231 |
| A3B (AA235-AA307) | LAAFKSDHPDLVLRIYTSRLYFHWRRKYQEGLC | |
| Peromyscus leucopus | NKKGKHAEILFIDEMRSLELGQARITCYLTWSPCPNCAQK | 232 |
| A3 (AA266-AA338) | LAAFKKDHPDLVLRVYTSRLYFHWRRKYQEGLC | |
| Mesocricetus auratus | NKKDKHAEILFIDKMRSLELCQVRITCYLTWSPCPNCAQE | 233 |
| A3 (AA268-AA340) | LAAFKKDHPDLVLRIYTSRLYFHWRRKYQEGLC | |
| Microtus ochrogaster | NKKGKHAEILFIDEMRSLKLSQERITCYLTWSPCPNCAQE | 234 |
| A3B (AA266-AA338) | LAAFKRDHPGLVLRIYASRLYFHWRRKYQEGLC | |
| Nannospalax galili | NKRAKHAEILLIDMMRSMELGQVQITCYITWSPCPTCAQE | 235 |
| A3 (AA231-AA302) | LAAFKQDHPDLVLRIYASRLYFHWKRKFQKGL | |
| Meriones | NKKGRHAEICLIDEMRSLGLGKAQITCYLTWSPCRKCAQE | 236 |
| unguiculatus A3 | LATEKKDHPDLVLRVYASRLYFHWSRKYQQGLC | |
| (AA233-AA305) | | |
| Dipodomys ordii A3 | NKKGHHAEIRFIERIRSMGLDPSQDYQITCYLTWSPCLDC | 237 |
| (AA256-AA330) | AFKLAKLKKDFPRLTLRIFTSRLYFHWIRKFQKGL | |
| Jaculus jaculus A3 | NKKGKHAEARFVDKMRSMQLDHALITCYLTWSPCLDCSQK | 238 |
| (AA303-AA374) | LAALKRDHPGLTLRIFTSRLYFHWVKKFQEGL | |
| Chinchilla lanigera | SPQKGHHAESRFIKRISSMDLDRSRSYQITCFLTWSPCPS | 239 |
| A3H (AA86-AA161) | CAQELASFKRAHPHLRFQIFVSRLYFHWKRSYQAGL | |
| Heterocephalus | KKGYHAESRFIKRICSMDLGQDQSYQVTCELTWSPCPHCA | 240 |
| glaber A3 (AA277- | QELVSFKRAHPHLRLQIFTARLFFHWKRSYQEGL | |
| AA350) | | |
| Octodon degus A3 | KKGQHAEIRFIERIHSMALDQARSYQITCELTWSPCPFCA | 241 |
| (AA256-AA329) | QELASFKSTHPRVHLQIFVSRLYFHWKRSYQEGL | |
| Urocitellus parryii | NKKGHHAEIRFIKKIRSLDLDQSQNYEVTCYLTWSPCPDC | 242 |
| A3 (AA256-AA330) | AQELVALTRSHPHVRLRLFTSRLYFHWEWSFQEGL | |
| Aotus nancymaae | NRHAEICFIDEIESMGLDKTQCYEVTCYLTWSPCPSCAQK | 243 |
| A3H (AA75-AA146) | LAAFTKAQVHLNLRIFASRLYYHWRSSYQKGL | |
| Cebus capucinus | NRHAEICFIDEIESMGLDKTQCYEVTCYLTWSPCPSCAQK | 244 |
| imitator A3H | LVAFAKAQDHLNLRIFASRLYYHWRRRYKEGL | |
| (AA55-AA126) | | |
| Saimiri boliviensis | HVEICFIDKIASMELDKTQCYDVTCYLTWSPCPSCAQKLA | 245 |
| boliviensis A3H | AFAKAQDHLNLRIFASRLYYHWRRSYQKGL | |
| (AA56-AA125) | | |
| Homo sapiens A3H | NKKKCHAEICFINEIKSMGLDETQCYQVTCYLTWSPCSSC | 246 |
| (AA49-AA123) | AWELVDFIKAHDHLNLGIFASRLYYHWCKPQQKGL | |
| Homo sapiens | ENKKKCHAEICFINEIKSMGLDETQCYQVTCYLTWSPCSS | 247 |
| ARP10 (AA48-AA123) | CAWELVDFIKAHDHLNLGIFASRLYYHWCKPQQKGL | |
| Pan paniscus A3H | NKKKCHAEICFINEIKSMGLDETQCYQVTCYLTWSPCSSC | 248 |
| (AA49-AA123) | AWKLVDFIQAHDHLNLRIFASRLYYHWCKPQQEGL | |
| Symphalangus | NKKKRHAEIRFINKIKSMGLDETQCYQVTCYLTWSPCPSC | 249 |
| syndactylus A3H | AWELVDFIKAHDHLNLGIFASRLYYHWCRHQQEGL | |
| (AA49-AA123) | | |
| Macaca mulatta A3H | NKKKDHAEIRFINKIKSMGLDETQCYQVTCYLTWSPCPSC | 250 |
| (AA49-AA123) | AGELVDFIKAHRHLNLRIFASRLYYHWRPNYQEGL | |
| Theropithecus gelada | NKKKEHAEIRFINKIKSMGLDETQCYQVTCYLTWSPCPSC | 251 |
| A3H (AA54-AA128) | AGKLVDFIKAHHHLNLRIFASRLYYHWRPNYQEGL | |
| Mandrillus | NKKKHHAEIHFINKIKSMGLDETQCYQVTCYLTWSPCPSC | 252 |
| leucophaeus A3H | ARELVDFIKAHRHLNLRIFASRLYYHWRPHYQEGL | |
| (AA49-AA123) | | |
| Bos grunniens A3 | NKKQRHAEIRFIDKINSLDLNPSQSYKIICYITWSPCPNC | 253 |
| (AA74-AA148) | ANELVNFITRNNHLKLEIFASRLYFHWIKPEKMGL | |
| Bubalus bubalis A3 | NKKQRHAEIRFIDKINSLDLNPSQSYKIICYITWSPCPNC | 254 |
| (AA74-AA148) | ASELVDFITRNDHLDLQIFASRLYFHWIKPFKRGL | |
| Odocoileus | NKKQRHAEIRFIDKINSLNLDRRQSYKIICYITWSPCPRC | 255 |
| virginianus texanus | ASELVDFITGNDHLNLQIFASRLYFHWKKPFQRGL | |
| A3H (AA209-AA283) | | |
| Sus scrofa A3 | NKKKRHAEIRFIDKINSLNLDQNQCYRIICYVTWSPCHNC | 256 |
| (AA51-AA125) | AKELVDFISNRHHLSLQLFASRLYFHWVRCYQRGL | |
| Ceratotherium simum | NKKKRHAEIRFIDKIKSLGLDRVQSYEITCYITWSPCPTC | 257 |
| simum A3B | ALELVAFTRDYPRLSLQIFASRLYFHWRRRSIQGL | |
| (AA232-AA306) | | |
| Equus caballus A3H | NKKKRHAEIRFIDKINSLGLDQDQSYEITCYVTWSPCATC | 258 |
| (AA79-AA153) | ACKLIKETRKFPNLSLRIFVSRLYYHWFRQNQQGL | |
| Enhydra lutris | KKKRHAEIRFIDSIRALQLDQSQRFEITCYLTWSPCPTCA | 259 |
| kenyoni A3B | KELAMEVQDHPHISLRLFASRLYFHWRWKYQEGL | |
| (AA243-AA316) | | |
| Leptonychotes | KKKRHAEIRFIDNIKALRLDTSQRFEITCYVTWSPCPTCA | 260 |
| weddellii A3H | KELVAFVRDHRHISLRLFASRLYFHWLRENKKGL | |
| (AA50-AA123) | | |
| Ursus arctos | NKKKRHAEIRFIDKIRSLQRDSSQTFEITCYVTWSPCFTC | 261 |
| horribilis A3F | AEELVAFVRDHPHVRLRLFASRLYFHWLRKYQEGL | |
| (AA552-AA626) | | |
| Panthera leo | NKKKRHAEICFIDKIKSLTRDTSQRFEIICYITWSPCPFC | 262 |
| bleyenberghi A3H | AEELVAFVKDNPHLSLRIFASRLYVHWRWKYQQGL | |
| (AA50-AA124) | | |
| Panthera tigris | NKKKRHAEICFIDKIKSLTRDTSQRFEIICYITWSPCPFC | 263 |
| sumatrae A3H | AEELVAFVKDNPHLSLRIFASRLYVHWRWKYQQGL | |
| (AA50-AA124) | | |
| Tupaia belangeri A3 | NKKHRHAEVRFIAKIRSMSLDLDQKHQLTCYLTWSPCPSC | 264 |
| (AA46-AA120) | AQELVTEMAESRHLNLQVFVSRLYEHWQRDEQQGL | |

TABLE B2
hA3FCDA1 Core Sequence Related Domains

| | | SEQ |
|---|---|---|
| | | ID |
| Name | Sequence | NO: |
| Pan troglodytes A3F | RRNTVWLCYEVKTKGPSRPRLDTKIFRGQVYFEPQYHAEMCELSWFCGNQ | 265 |
| (AA29-AA136) | LPAYKCFQITWFVSWTPCPDCVAKLAEFLAEHPNVTLTISAARLYYYWER | |
| | DYRRALCR | |
| Pan paniscus A3F | RRNTVWLCYEVKTKGPSRPRLDTKIFRGQVYFQFENHAEMCELSWFCGNQ | 266 |
| (AA29-AA136) | LPAYKCFQITWFVSWTPCPDCVAKLAEFLAEHPNVTLTISAARLYYYWER | |
| | DYRRALCR | |
| Colobus angolensis | RRNTVWLCYEVKTRGPSMPTWGAKIFRGQVYFEPQYHAEMCFLSWFCGNQ | 267 |
| palliatus A3F | LPAYKCFQITWFVSWTPCPDCVGKVAEFLAEHPNVTLTISAARLYYYWET | |
| (AA29-AA136) | DYRRALCR | |
| Macaca mulatta A3F | RRNTVWLCYEVKTRGPSMPTWDTKIFRGQVYSKPEHHAEMCELSRECGNQ | 268 |
| (AA29-AA136) | LPAYKRFQITWFVSWTPCPDCVAKVAEFLAEHPNVTLTISAARLYYYWET | |
| | DYRRALCR | |
| Macaca fascicularis | RRNTVWLCYEVKTRGPSVPTWGTKIFRGQVYSKPEHHAEMCFLSWECGNQ | 269 |
| A3F | LPTYKRFQITWFVSWTPCPDCVAKVAEFLAEHPNVILTISAARLYYYWET | |
| (AA29-AA136) | DYRRALCR | |
| Rhinopithecus | RRNTVWLCYEVKTRGPSMPTWGAKIFRGQVYFEPQYHAEMCELSWFCGNQ | 270 |
| roxellana A3F | LPAYKRFQITWFVSWTPCPDCVAKVAEFLAEHPNVTLTISAARLYYYWET | |
| (AA29-AA136) | DYRRALCR | |
| Rhinopithecus bieti | RRNTVWLCYEVKTRGPSMPTWGAKIFRGQVYFEPQYHAEMCELSWFCGNQ | 271 |
| A3F | LPAYKRFQITWFVSWTPCPDCVAKVAEFLAEHPNVILTISAARLYYYWET | |
| (AA18-AA125) | DYRRALCR | |
| Rhinopithecus | RRNTVWLCYEVKTRGPSMPTWGAKIFRGQVYFEPQYHAEMCELSWFCGNQ | 272 |
| roxellana A3F | LPAYKRFQITWFVSWTPCPDCVAKVAEFLAEHPNVILTISAARLYYYWET | |
| (AA29-AA136) | DYRRALCR | |
| Macaca mulatta A3F | RRNTVWLCYEVKTRGPSMPTWDTKIFRGQVYSKPEHHAEMCFLSRFCGNQ | 273 |
| (AA40-AA147) | LPAYKRFQITWFVSWTPCTDCVAKVAEFLAEHPNVILTISAARLYYYWET | |
| | DYRRALCR | |
| Trachypithecus | RRNTVWLCYEVKTRGPSMPTWGAKIFRGQVYFEPQYHAEMCELSWFCGNQ | 274 |
| francoisi A3F | LPAYKRFRITWFVSWTPCPDCVAKVAEFLAEHPNVTLTISAARLYYYWET | |
| (AA40-AA147) | DYRRALCR | |
| Gorilla gorilla A3F | RRNTVWLCYEVKTKGPSRPPLDAKIFRGQVYFEPQYHAEMCELSWFCGNQ | 275 |
| (AA29-AA127) | LPAYKCFQITWFVSWTPCPDCVAKLAEFLAEHPNVTLTISAARLYYYWE | |
| Papio anubis A3F | RRNTVWLCYEVKTRGPSMPTWDAKIFRGQVYFQPQYHAEMCELSRFCGNQ | 276 |
| (AA29-AA136) | LPAYKRFQITWFVSWTPCPDCVVKVTEFLAEHPNVILTISAARLYYYWET | |
| | DYRRALCR | |
| Pongo abelii A3F | RRNTVWLCYKVKTKGPSRPPLNAKIFRGQVYFEPQYHAEMCELSWECGNQ | 277 |
| (AA29-AA136) | LSAYERFQITWFVSWTPCPDCVAMLAEFLAEHPNVTLTVSAARLYYYWER | |
| | DYRGALRR | |
| Macaca leonina A3F | RRNTVWLCYEVKTRGPSMPTWGTKIFRGQVCFEPQYHAEMCELSRFCGNQ | 278 |
| (AA29-AA136) | LPAYKRFQITWFVSWTPCPDCVAKVAEFLAEHPNVTLTISAARLYYYWET | |
| | DYRRALCR | |
| Macaca nemestrina | RRNTVWLCYEVKTRGPSMPTWGTKIFRGQVCFEPQYHAEMCELSRFCGNQ | 279 |
| A3F | LPAYKRFQITWFVSWTPCPDCVAKVAEFLAEHPNVTLTISAARLYYYWET | |
| (AA29-AA136) | DYRRALCR | |
| Homo sapiens A3B | RSYTWLCYEVKIKRGRSNLLWDTGVFRGQVYFEPQYHAEMCFLSWFCGNQ | 280 |
| (AA30-AA137) | LPAYKCFQITWFVSWTPCPDCVAKLAEFLSEHPNVTLTISAARLYYYWER | |
| | DYRRALCR | |
| Gorilla gorilla | RSYNWLCYEVKIKRGRSNLLWNTGVFRGQMYSQPEHHAEMCFLSWFCGNQ | 281 |
| gorill aA3B | LPAYKCFQITWFVSWTPCPDCVAKLAEFLAEYPNVTLTISAARLYYYWER | |
| (AA30-AA137) | DYRRALCR | |
| Pan troglodytes A3B | RSYTWLCYEVKIRRGHSNLLWDTGVERGQMYSQPEHHAEMCELSWECGNQ | 282 |
| (AA30-AA137) | LSAYKCFQITWFVSWTPCPDCVAKLAKFLAEHPNVTLTISAARLYYYWER | |
| | DYRRALCR | |
| Theropithecus | RRNTVWLCYEVKTRGPSMPTWGTKIFRGQVYFQPQYHAEMCELSRFCGNQ | 283 |
| gelada A3F | LPAYKRFQITWFVSWNPCPDCVAKVIEFLAEHPNVTLTISAARLYYYWGR | |
| (AA29-AA136) | DWRRALRR | |
| Mandrillus | RRNTVWLCYKVKTRGPSMPTWGTKIFRGQVYFQPQYHAEMCELSWFCGNQ | 284 |
| leucophaeus A3F | LPAYKRFQITWFVSWTPCPDCVVKVAEFLAEHPNVTLTISAARLYYYWET | |
| (AA29-AA130) | DY | |
| Gorilla gorilla | RSYTWLCYEVKIKRGRSNLLWDTGVFRGQMYSQPEHHAEMCELSWFCGNQ | 285 |
| gorilla A3B | LPAYKCFQITWFVSWTPCLDCVAKLAEFLAEYPNVILTISTARLYYYWER | |
| (AA30-AA137) | DYRRALCR | |
| Pan paniscus A3B | RSYTWLCYEVKIRRGHSNLLWDTGVFRGQMYSQPEHHAEMYFLSWECGNQ | 286 |
| (AA30-AA137) | LSAYKCFQITWFVSWTPCPDCVAKLAEFLAEHPNVTLTISAARLYYYWER | |
| | DYRRALCR | |
| Hylobates moloch | RSYTWLCYEVKIRKDPSKLPWDTGVFRGQMYFQPEYHAEMCELSWFCGNQ | 287 |
| A3B | LPAYKRFQITWFVSWTPCPDCVAKVAEFLAEHPNVILTISAARLYYYWEK | |
| (AA30-AA137) | DWQRALCR | |
| Symphalangus | RRNTVWLCYEVKTKDPSRPRLDTKIFRGKVYFQLENHAEMCELSWFCGNQ | 288 |
| syndactylus A3G | LPANRCFQITWFVSWNPCLPCVAKVTKFLAEHPNVTLTISAARLYYYRAR | |
| (AA22-AA129) | DWRRALRR | |
| Macaca mulatta A3B | RSYTWLCYEVKIRKDPSKLPWDTGVFRGQMYSKPEHHAEMCELSWFCGNQ | 289 |
| (AA30-AA137) | LPAHKRFQITWFVSWTPCPDCVAKVAEFLAEYPNVTLTISAARLYYYWET | |
| | DYRRALCR | |
| Chlorocebus sabaeus | RSYTWLCYEVKIRKDPSKLPWDTGVFRGQMYSKPEHHAEMCELSWFCGNQ | 290 |
| A3B | LPAHKRFQITWFVSWTPCPDCVAKVAEFLAEYPNVTLTISAARLYYYWET | |
| (AA30-AA137) | DYRRALCR | |
| Nomascus | RRSYTWLCYEVKIRKDPSKLPWDTGVFRGQMYFQPEYHAEMCELSWFCGN | 291 |
| leucogenys A3B | QLPAYKRFQITWFVSWTPCPDCVAKVAVELAEHPNVTLTISAARLYYYWE | |
| (AA30-AA137) | KDWQRALCR | |
| Trachypithecus | RSYTWLCYEVKIRKDPSKLPWDTGVFRGQVYSEPEHHAEMYFLSWECGNQ | 292 |
| francoisi A3B | LPAYKRFWITWFVSWTPCPDCVAKLAEFLTEHPNVTLTISAARLYYYRGR | |
| (AA30-AA137) | DWRRALCR | |
| Trachypithecus | RSYTWLCYEVKKRKDPSKLPWDTGVFRGQVYSEPEHHAEMYELSWFCGNQ | 293 |
| francoisi A3F | LPAYKRFWITWFVSWTPCPDCVAKVAEFLAEHPKVILTISAARLYYYWDR | |
| (AA22-AA129) | DWRRALCR | |
| Rhinopithecus bieti | RSYTWLCYEVKIRKDPSKLPWDTGVERGQVYSEPEHHAEMYELSWFCGNQ | 294 |
| A3F | LPAYKRFQITWFVSWTPCPDCVAKVAEFLTEHPNVILTISAARLYYYRGR | |
| (AA30-AA137) | DWRRALCR | |
| Rhinopithecus | RSYTWLCYEVKIRKDPSKLPWDTGVERGQVYSEPEHHAEMYELSWECGNQ | 295 |
| roxellana A3B | LPAYKRFQITWFVSWTPCPDCVAKVAEFLTEHPNVTLTISAARLYYYRGR | |
| (AA30-AA137) | DWRRALCR | |
| Pongo abelii A3F | RRNYTWLCYEVKIRKDPSKLAWDTGVFRGQVYSQPEHHAEMCELSWFCGN | 296 |
| (AA29-AA128) | QLSAYERFQITWFVSWTPCPDCVAKLAEFLAEHPNVTLTVSAARLYYYWE | |
| Macaca mulatta A3B | RSYTWLCYEVKIRKDPSKLPWDTGVFRGQVYSKPEHHAEMCELSRFCGNQ | 297 |
| (AA30-AA137) | LPAYKRFQITWFVSWNPCPDCVAKVIEFLAEHPNVTLTISTARLYYYWGR | |
| | DWQRALCR | |
| Macaca leonina A3B | RSYTWLCYEVKIRKDPSKLPWDTGVFRGQVYSKPEHHAEMCELSRFCGNQ | 298 |
| (AA30-AA137) | LPAYKRFQITWFVSWNPCPDCVVKVIEFLAEHPNVTLTISTARLYYYWGR | |
| | DWQRALCR | |
| Macaca nemestrina | RSYTWLCYEVKIRKDPSKLPWDTGVFRGQVYSKPEHHAEMCELSRFCGNQ | 299 |
| A3B | LPAYKRFQITWFVSWNPCPDCVAKVTEFLAEHPNVTLTISTARLYYYWGR | |
| (AA30-AA137) | DWQRALCR | |
| Macaca mulatta A3D | RSYTWLCYEVKIRKDPSKLPWDTGVFRGQVYFQPQYHAEMCELSWFCGNQ | 300 |
| (AA30-AA137) | LPAYKRFQITWFVSWNPCPDCVAKVTEFLAEHPNVTLTISVARLYYYRGK | |
| | DWRRALCR | |
| Pongo abelii A3F | RRNYTWLCYEVKIRKDPSKLAWDTGVFRGQVLPKLQSNHRREVYFEPQYH | 301 |
| (AA29-AA149) | AEMCFLSWFCGNQLSAYERFQITWFVSWTPCPDCVAMLAEFLAEHPNVTL | |
| | TVSAARLYYYWERDYRGALRR | |
| Erythrocebus patas | RRYTWLCYEVKIKKDPSKLPWDTGVFQGQVRPKFQSNRRYEVYFQPQYHA | 302 |
| A3DE | EMCFLSWFCGNQLPAYKHFQITWFVSWNPCPDCVAKVTEFLAEHPNVTLT | |
| (AA30-AA149) | ISAARLYYYWGKDWRRALCR | |
| Pan troglodytes A3B | MYSQPEHHAEMCFLSWFCGNQLSAYKCFQITWFVSWTPCPDCVAKLAKEL | 303 |
| (AA1-AA79) | AEHPNVTLTISAARLYYYWERDYRRALCR | |
| Macaca mulatta | RSYTWLCYEVKIRKDPSKLPWDTGVERGQVRPKLQSNRRYEVYFQPQYHA | 304 |
| A3DE | EMCFLSWFCGNQLPAYKRFQITWEVSWNPCPDCVAKVTEFLAEHPNVTLT | |
| (AA30-AA149) | ISAARLYYYWGKDWRRALRR | |
| Piliocolobus | RRYTWLCYEVKIMKDHSKLPWYTGVERGQVYFEPQNHAEMCELSWFCGNQ | 305 |
| tephrosceles A3F | LPAYECCQITWFVSWTPCPDCVAKVTEFLAEHPNVILTISAARLYYYRGR | |
| (AA30-AA137) | DWRRALRR | |
| Macaca leonina A3D | RSYTWLCYEVKIRKDPSKLPWYTGVFRGQVYFQPQYHAEMCELSWFCGNQ | 306 |
| (AA30-AA137) | LPANKRFQITWFVSWNPCPDCVAKVTEFLAEHPNVTLTISVARLYYYRGK | |
| | DWRRALRR | |
| Macaca nemestrina | RSYTWLCYEVKIRKDPSKLPWDTGVERDQVYFQPQYHAEMCELSWFCGNQ | 307 |
| A3D | LPANKRFQITWFVSWNPCPDCVTKVTEFLAEHPNVILTISVARLYYYRGK | |
| (AA30-AA137) | DWRRALRR | |
| Chlorocebus | RRYTWLCYEVKIKKDPSKLPWDTGVFPGQVRPKFQSNRRYEVYFQPQYHA | 308 |
| aethiops A3DE | EMYFLSWFCGNQLPAYKHFQITWFVSWNPCPDCVAKVTEFLAEHRNVTLT | |
| (AA30-AA149) | ISAARLYYYWGKDWRRALCR | |
| Macaca mulatta | RSYTWLCYEVKIRKDPSKLPWDTGVFRGQVRPKLQSNRRYELSNWECRKH | 309 |
| A3D | VYFQPQYHAEMCFLSWFCGNQLPANKRFQITWFVSWNPCPDCVAKVTEFL | |
| (AA30-AA158) | AEHPNVTLTISAARLYYYWGKDWRRALRR | |

TABLE B3
hA3BCDA1-Related Domains

| | | SEQ |
|---|---|---|
| | | ID |
| Name | Sequence | NO: |
| Gorilla A3B (AA29- | GRSYNWLCYEVKIKRGRSNLLWNTGVFRGQMYSQPEHHAEMCELSWFCGN | 310 |
| AA138) | QLPAYKCFQITWFVSWTPCPDCVAKLAEFLAEYPNVTLTISTARLYYYWE | |
| | RDYRRALCRL | |
| Pan paniscus A3B | GRSYTWLCYEVKIRRGHSNLLWDTGVFRGQMYSQPEHHAEMYFLSWFCGN | 311 |
| (AA29-AA138) | QLPAYKCFQITWFVSWTPCPDCVAKLAEFLAEHPNVTLTISAARLYYYWE | |
| | RDYRRALCRL | |
| Pan troglodytes A3B | GRSYTWLCYEVKIRRGHSNLLWDTGVFRGQMYSQPEHHAEMCELSWFCGN | 312 |
| (AA29-AA138) | QLSAYKCFQITWFVSWTPCPDCVAKLAKFLAEHPNVTLTISAARLYYYWE | |
| | RDYRRALCRL | |
| Gorilla A3F (AA30- | RNTVWLCYEVKTKGPSRPPLDAKIFRGQVYFEPQYHAEMCELSWFCGNQL | 313 |
| AA137) | PAYKCFQITWFVSWTPCPDCVAKLAEFLAEHPNVTLTISAARLYYYWE | |
| Pan troglodytes A3F | RNTVWLCYEVKTKGPSRPRLDTKIFRGQVYFEPQYHAEMCELSWFCGNQL | 314 |
| (AA30-AA137) | PAYKCFQITWFVSWTPCPDCVAKLAEFLAEHPNVTLTISAARLYYYWERD | |
| | YRRALCRL | |
| Human sapiens A3F | RNTVWLCYEVKTKGPSRPRLDAKIFRGQVYSQPEHHAEMCFLSWFCGNQL | 315 |
| (AA30-AA137) | PAYKCFQITWFVSWTPCPDCVAKLAEFLAEHPNVILTISAARLYYYWERD | |
| | YRRALCRL | |
| Macaca leonine | RNTVWLCYEVKTRGPSMPTWGTKIFRGQVCFEPQYHAEMCELSRFCGNQL | 316 |
| A3F (AA30-AA137) | PAYKRFQITWFVSWTPCPDCVAKVAEFLAEHPNVTLTISAARLYYYWETD | |
| | YRRALCRL | |
| Macaca nemestrina | RNTVWLCYEVKTRGPSMPTWGTKIFRGQVCFEPQYHAEMCELSRFCGNQL | 317 |
| A3F (AA30-AA137) | PAYKRFQITWFVSWTPCPDCVAKVAEFLAEHPNVTLTISAARLYYYWETD | |
| | YRRALCRL | |
| Rhinopithecus | RNTVWLCYEVKTRGPSMPTWGAKIFRGQVYFEPQYHAEMCELSWFCGNQL | 318 |
| roxellana A3F | PAYKRFQITWFVSWTPCPDCVAKVAEFLAEHPNVTLTISAARLYYYWETD | |
| (AA30-AA137) | YRRALCRL | |
| Mandrillus | RNTVWLCYKVKTRGPSMPTWGTKIFRGQVYFQPQYHAEMCELSWFCGNQL | 319 |
| leucophaeus A3F | PAYKRFQITWFVSWTPCPDCVVKVAEFLAEHPNVTLTISAARLYYYWETD | |
| (AA30-AA130) | Y | |
| Macaca mulatta A3F | RNTVWLCYEVKTRGPSMPTWDTKIFRGQVYSKPEHHAEMCELSRFCGNQL | 320 |
| (AA30-AA137) | PAYKRFQITWFVSWTPCPDCVAKVAEFLAEHPNVTLTISAARLYYYWETD | |
| | YRRALCRL | |
| Theropithecus | RNTVWLCYEVKTRGPSMPTWGTKIFRGQVYFQPQYHAEMCFLSRFCGNQL | 321 |
| gelada A3F | PAYKRFQITWFVSWNPCPDCVAKVIEFLAEHPNVTLTISAARLYYYWGRD | |
| (AA30-AA137) | WRRALRRL | |
| Cercocebus atys A3B | GRSYTWLCYEVKIRKDPSKLPWYTGVFRGQVYSKPEHHAEMCELSRFCGN | 322 |
| (AA29-AA138) | QLPAYKRFQITWFVSWNPCPDCVAKVIEFLAEHPNVTLTISAARLYYYWS | |
| | RDWQRALCRL | |
| Macaca fascicularis | GRSYTWLCYEVKIRKDPSKLPWDTGVFRGQVYSKPEHHAEMCELSRFCGN | 323 |
| A3B (AA29-AA138) | QLPAYKRFQITWFVSWNPCPDCVAKVIEFLAEHPNVILTISTARLYYYWG | |
| | RDWQRALCRL | |
| Macaca mulatta A3B | GRSYTWLCYEVKIRKDPSKLPWDTGVERGQVYSKPEHHAEMCFLSRFCGN | 324 |
| (AA29-AA138) | QLPAYKRFQITWEVSWNPCPDCVAKVIEFLAEHPNVTLTISTARLYYYWG | |
| | RDWQRALCRL | |
| Macaca leonina A3B | GRSYTWLCYEVKIRKDPSKLPWDTGVFRGQVYSKPEHHAEMCFLSRFCGN | 325 |
| (AA29-AA138) | QLPAYKRFQITWFVSWNPCPDCVVKVIEFLAEHPNVILTISTARLYYYWG | |
| | RDWQRALCRL | |
| Mandrillus | GRSYTWLCYEVKIRKDPSKLPWYTGVFRGQVYSKPEHHAEMCELSRFCGN | 326 |
| leucophaeus A3B | QLPAYKRFQITWFVSWNPCPDCVAKVIEFLAEHPNVILTIFTARLYYYWG | |
| (AA29-AA138) | RDWQRALCRL | |
| Macaca nemestrina | GRSYTWLCYEVKIRKDPSKLPWDTGVFRGQVYSKPEHHAEMCELSRFCGN | 327 |
| A3B (AA29-AA138) | QLPAYKRFQITWFVSWNPCPDCVAKVTEFLAEHPNVTLTISTARLYYYWG | |
| | RDWQRALCRL | |
| Rhinopithecus bieti | GRSYTWLCYEVKIRKDPSKLPWDTGVFRGQVYSEPEHHAEMYELSWFCGN | 328 |
| A3F (AA29-AA138) | QLPAYKRFQITWFVSWTPCPDCVAKVAEFLTEHPNVTLTISAARLYYYRG | |
| | RDWRRALCRL | |
| Rhinopithecus | GRSYTWLCYEVKIRKDPSKLPWDTGVERGQVYSEPEHHAEMYFLSWFCGN | 329 |
| roxellana A3B | QLPAYKRFQITWFVSWTPCPDCVAKVAEFLTEHPNVTLTISAARLYYYRG | |
| (AA29-AA138) | RDWRRALCRL | |
| Chlorocebus sabaeus | GRSYTWLCYEVKIRKDPSKLPWDTGVERGQMYSKPEHHAEMCELSWFCGN | 330 |
| A3B (AA29-AA138) | QLPAHKRFQITWFVSWTPCPDCVAKVAEFLAEYPNVILTISAARLYYYWE | |
| | TDYRRALCRL | |
| Nomascus | RSYTWLCYEVKIRKDPSKLPWDTGVFRGQMYFQPEYHAEMCELSWFCGNQ | 331 |
| leucogenys A3B | LPAYKRFQITWFVSWTPCPDCVAKVAVELAEHPNVTLTISAARLYYYWEK | |
| (AA30-AA138) | DWQRALCRL | |
| Cercocebus atys A3F | GRSYTWLCYEVKIKKYPSKLLWDTGVFQGQVYFQPQYHAEMCELSRFCGN | 332 |
| (AA29-AA138) | QLPAYKRFQITWFVSWNPCPDCVAKVTEFLAEHPNVTLTISAARLYYYWE | |
| | KDXRRALRRL | |
| Papio anubis A3F | GRSYTWLCYEVKIKEDPSKLLWDTGVFQGQVYFQPQYHAEMCELSRFCGN | 333 |
| (AA29-AA138) | QLPAYKRFQITWFVSWNPCPDCVAKVTEFLAEHPNVTLTISAARLYYYWG | |
| | RDWRRALRRL | |
| Chlorocebus | GRRYTWLCYEVKIKKDPSKLPWDTGVFPGQVRPKFQSNRRYEVYFQPQYH | 334 |
| aethiops A3D | AEMYFLSWFCGNQLPAYKHFQITWFVSWNPCPDCVAKVTEFLAEHRNVTL | |
| (AA29-AA150) | TISAARLYYYWGKDWRRALCRL | |
| Chlorocebus sabaeus | GRRYTWLCYEVKIKKDPSKLPWDTGVFPGQPQYHAEMYFLSWFCGNQLPA | 335 |
| A3D (AA29-AA134) | YKHFQITWFVSWNPCPDCVAKVTEFLAEHRNVTLTISAARLYYYWGKDWR | |
| | RALCRL | |
| Chlorocebus sabaeus | GRRYTWLCYEVKIKKDPSKLPWDTGVFPGQVRPKFQSNRRQKVYFQPQYH | 336 |
| A3F (AA29-AA150) | AEMYFLSWFCGNQLPAYKHFQITWFVSWNPCPDCVAKVTEFLAEHRNVTL | |
| | TISAARLYYYWGKDWRRALCRL | |
| Erythrocebus patas | GRRYTWLCYEVKIKKDPSKLPWDTGVFQGQVRPKFQSNRRYEVYFQPQYH | 337 |
| A3D (AA29-AA150) | AEMCFLSWFCGNQLPAYKHFQITWFVSWNPCPDCVAKVTEFLAEHPNVTL | |
| | TISAARLYYYWGKDWRRALCRL | |
| Macaca fascicularis | GRSYTWLCYEVKIRKDPSKLPWDTGVFRGQVRPKLQSNRRYELSNWECRK | 338 |
| A3D (AA29-AA159) | RVYFQPQYHAEMYFLSWFCGNQLPANKRFQITWFASWNPCPDCVAKVTEF | |
| | LAEHPNVTLTISVARLYYYRGKDWRRALRRL | |
| Macaca fascicularis | GRSYTWLCYEVKIRKDPSKLPWDTGVFRGQVYFQPQYHAEMYFLSWFCGN | 339 |
| A3F (AA29-AA138) | QLPANKRFQITWFASWNPCPDCVAKVTEFLAEHPNVILTISVARLYYYRG | |
| | KDWRRALRRL | |
| Macaca nemestrina | GRSYTWLCYEVKIRKDPSKLPWDTGVFRDQVYFQPQYHAEMCELSWFCGN | 340 |
| A3D (AA29-AA138) | QLPANKRFQITWFVSWNPCPDCVTKVTEFLAEHPNVILTISVARLYYYRG | |
| | KDWRRALRRL | |
| Macaca leonina A3D | GRSYTWLCYEVKIRKDPSKLPWYTGVFRGQVYFQPQYHAEMCELSWFCGN | 341 |
| (AA29-AA138) | QLPANKRFQITWFVSWNPCPDCVAKVTEFLAEHPNVILTISVARLYYYRG | |
| | KDWRRALRRL | |
| Macaca mulatta A3D | GRSYTWLCYEVKIRKDPSKLPWDTGVFRGQVYFQPQYHAEMCFLSWFCGN | 342 |
| (AA29-AA138) | QLPAYKRFQITWFVSWNPCPDCVAKVTEFLAEHPNVTLTISVARLYYYRG | |
| | KDWRRALCRL | |
| Gorilla A3D (AA29- | GRSYTWLCYEVKIRRGSSNLLWNTGVFRGPVPPKLQSNHRQEVYFQFENH | 343 |
| AA150) | AEMCFLSWFCGNRLPANRRFQITWFVSWNPCLPCVVKVTKFLAEHPNVTL | |
| | TISAARLYYYRDREWRRVLRRL | |
| Pan paniscus A3D | GRSYTWLCYEVKIKRGCSNLIWDTGVFRGPVLPKLQSNHRQEVYFQFENH | 344 |
| (AA29-AA150) | AEMCFFSWFCGNRLPANRRFQITWFVSWNPCLPCVVKVTKFLAEHPNVTL | |
| | TISAARLYYYQDREWRRVLRRL | |
| Pan troglodytes A3D | GRSYTWLCYEVKIKRGCSNLIWDTGVERGPVLPKLQSNHRQEVYFQFENH | 345 |
| (AA29-AA150) | AEMCFFSWFCGNRLPANRRFQITWFVSWNPCLPCVVKVTKFLAEHPNVTL | |
| | TISAARLYYYQDREWRRVLRRL | |
| Homo sapiens A3D | GRSYTWLCYEVKIKRGRSNLLWDTGVFRGPVLPKRQSNHRQEVYFRFENH | 346 |
| (AA29-AA150) | AEMCFLSWFCGNRLPANRRFQITWFVSWNPCLPCVVKVTKFLAEHPNVTL | |
| | TISAARLYYYRDRDWRWVLLRL | |
| Nomascus | GRSYTWLCYEVKIRKDPSKLPWDKGVFRGQVLPKFQSNHRQEVYFQLENH | 347 |
| leucogenys A3D | AEMCELSWFCGNQLPANRRFQITWFVSWNPCLPCVAKVTEFLAEHPNVTL | |
| (AA29-AA150) | TISAARLYYYRGRDWRRALRRL | |
| Saimiri boliviensis | GKKYTWLCYEVKIKKDTSKLPWNTGVFRGQVNENPEHHAEMYELSWERGK | 348 |
| A3C (AA29-AA138) | LLPACKRSQITWFVSWNPCLYCVAKVAEFLAEHPNVTLTVSTARLYCYWK | |
| | KDWRRALRKL | |
| Saimiri boliviensis | GKKYTWLCYEVKIKKDTSKLPWNTGVFRGQVNENPEHHAEMYFLSWERGK | 349 |
| A3F (AA29-AA138) | LLPACKRSQITWFVSWNPCLYCVAKVAEFLAEHPNVTLTVSTARLYCYWK | |
| | KDWRRALRKL | |
| Piliocolobus | GRRYTWLCYEVKIMKDHSKLPWYTGVFRGQVYFEPQNHAEMCELSWFCGN | 350 |
| tephrosceles A3F | QLPAYECCQITWFVSWTPCPDCVAKVTEFLAEHPNVTLTISAARLYYYRG | |
| (AA36-AA145) | RDWRRALRRL | |
| Colobus angolensis | GRRYTWLCYEVKISKDPSKLPWDTGIFRGQVYFEPQYHAEMCELSWYCGN | 351 |
| palliatus A3F | QLPAYKCFQITWFVSWTPCPDCVGKVAEFLAEHPNVTLTISAARLYYYWE | |
| (AA29-AA138) | TDYRRALCRL | |
| Pongo abelii A3F | RNYTWLCYEVKIRKDPSKLAWDIGVERGQVLPKLQSNHRREVYFEPQYHA | 352 |
| (AA30-AA150) | EMCFLSWFCGNQLSAYERFQITWFVSWTPCPDCVAMLAEFLAEHPNVTLT | |
| | VSAARLYYYWERDYRGALRRL | |

In some embodiments, the protease cleavage site is a self-cleaving peptide, such as the 2A peptides. “2A peptides” are 18-22 amino-acid-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. A few non-limiting examples of 2A peptides are provided in SEQ ID NO:217-219.

In some embodiments, the protease cleavage site is a cleavage site (e.g., SEQ ID NO:196) for the TEV protease. In some embodiments, the TEV protease provided in the base editing system includes two separate fragments, each of which on its own is not active. However, in the presence of the remaining fragment of the TEV protease, they will be able to execute the cleavage. Such an arrangement provides additional control and flexible of the base editing capabilities. The TEV fragments may be the TEV N-terminal domain (e.g., SEQ ID NO:194) or the TEV C-terminal domain (e.g., SEQ ID NO:195).

Various arrangement of the proteins/fragments can be made for a fusion protein in the disclosed base editing systems. Non-limiting examples include, from N-terminal side to C-terminal side:
(1) first fragment (e.g., catalytic domain)-protease cleavage site-second fragment (e.g., inhibitory domain);
(2) first fragment (e.g., catalytic domain and Cas protein)-protease cleavage site-second fragment (e.g., inhibitory domain);
(3) first fragment (e.g., catalytic domain, Cas protein and TEV N-terminal domain)-protease cleavage site (e.g., TEV cleavage site)-second fragment (e.g., inhibitory domain);
(4) second fragment (e.g., inhibitory domain)-protease cleavage site (e.g., TEV cleavage site)-first fragment (e.g., catalytic domain, Cas protein and TEV N-terminal domain); and
(5) second fragment (e.g., inhibitory domain)-protease cleavage site (e.g., TEV cleavage site)-first fragment (e.g., Cas protein, catalytic domain, and TEV C-terminal domain).

Such fusion proteins may include other fragments, such as uracil DNA glycosylase inhibitor (UGI) and nuclear localization sequences (NLS).

The “Uracil Glycosylase Inhibitor” (UGI), which can be prepared from Bacillus subtilis bacteriophage PBS1, is a small protein (9.5 kDa) which inhibits E. coli uracil-DNA glycosylase (UDG) as well as UDG from other species. Inhibition of UDG occurs by reversible protein binding with a 1:1 UDG:UGI stoichiometry. UGI is capable of dissociating UDG-DNA complexes. A non-limiting example of UGI is found in Bacillus phage AR9 (YP_009283008.1). In some embodiments, the UGI comprises the amino acid sequence of SEQ ID NO:216 or has at least at least 70%, 75%, 80%, 85%, 90% or 95% sequence identity to SEQ ID NO:216 and retains the uracil glycosylase inhibition activity.

The fusion protein, in some embodiments, may include one or more nuclear localization sequences (NLS).

A “nuclear localization signal or sequence” (NLS) is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A non-limiting example of NLS is the internal SV40 nuclear localization sequence (iNLS).

In some embodiments, a peptide linker is optionally provided between each of the fragments in the fusion protein. In some embodiments, the peptide linker has from 1 to 100 amino acid residues (or 3-20, 4-15, without limitation). In some embodiments, at least 10%, 20%, 30%, 40%, 50%, 60%, 70%, 80% or 90% of the amino acid residues of peptide linker are amino acid residues selected from the group consisting of alanine, glycine, cysteine, and serine.

Nucleobase Deaminase Inhibitors Fusion Proteins, tBEs

As demonstrated in the experimental examples, hA3F-CDA1 has been identified as an excellent cytidine deaminase inhibitor. Analogs of hA3F-CDA1 are shown in Table B2, as well as those having at least 70%, 75%, 80%, 85% 90%, 95%, 97%, 98%, or 99% sequence identity to hA3F-CDA1 or any of those in Table B2.

Accordingly, a fusion protein is designed that can be used to generate a base editor with improved base editing specificity and efficiency. In one embodiment, the present disclosure provides a fusion protein that includes a first fragment comprising a nucleobase deaminase (e.g., a cytidine deaminase) or a catalytic domain thereof, a second fragment comprising a nucleobase deaminase inhibitor, and a protease cleavage site between the first fragment and the second fragment. In some embodiments, the nucleobase deaminase inhibitor is hA3F-CDA1 (SEQ ID NO:192), or any of its analogs, such as those shown in Table B2, as well as those having at least 70%, 75%, 80%, 85% 90%, 95%, 97%, 98%, or 99% sequence identity to hA3F-CDA1 or any of those in Table B2.

A base editor that incorporates such a fusion protein has reduced or even no editing capability and accordingly will generate reduced or no off-target mutations. Upon cleavage of the protease cleavage site and release of the nucleobase deaminase inhibitor from the fusion protein at a target site, the base editor that is at the target site will then be able to edit the target site efficiently.

In some embodiments, the fusion protein further includes a clustered regularly interspaced short palindromic repeats (CRISPR)-associated (Cas) protein, optionally in the first fragment, next to the nucleobase deaminase or the catalytic domain thereof.

When the fusion protein is used, in vitro, ex vivo, or in vivo, to conduct gene/base editing in a cell, two additional molecules can be introduced. In one example, one molecule (B) is a single guide RNA (sgRNA) that further incorporates a tag sequence that can be recognized by an RNA recognition peptide. The sgRNA, alternatively, can be replaced by a crRNA that targets the target site and a CRISPR RNA (crRNA) alone, or in combination with a trans-activating CRISPR RNA (tracrRNA). Examples of tag sequences and corresponding RNA recognition peptides include MS2/MS2 coat protein (MCP), PP7/PP7 coat protein (PCP), and boxB/boxB coat protein (N22p), the sequences of which are provided herein. The molecule (B) may be provided as a DNA sequence encoding the RNA molecule.

The other additional molecule (C), in some embodiments, includes a second TEV protease fragment coupled to the RNA recognition peptide (e.g., MCP, PCP, N22p). The first TEV fragment and the second TEV fragment, in some embodiments, when present together, are able to cleave a TEV protease site.

Such co-presence can be triggered by the molecule (C) binding to the molecule (B) by virtue of the tag sequence-RNA recognition protein interaction. Meanwhile, the fusion protein (A) and the molecule (B) will be both present at the target genome locus for gene editing. Therefore, the molecule (B) brings both of the TEV protease fragments from the fusion protein (A) and molecule (C) together, which will activate the TEV protease, leading to removal of the nucleobase deaminase inhibitor from the fusion protein and activation of the base editor. It can be readily appreciated that such activation only occurs at the target genome site, not at off-target single-stranded DNA regions. As such, base editing does not occur at the single-stranded DNA regions that sgRNA does not bind to.

### Gene Therapy

The disclosed base editing system can be used to engineer a target cell. If used in vitro or ex vivo, the gene therapy approach can increase the expression of γ-globin in the target cell. If used in vivo, the gene therapy approach can treat diseases associated with insufficient production or dysfunction of hemoglobins. Example diseases include β-thalassemia, sickle cell anemia, Haemoglobin C, and Haemoglobin E.

In some embodiments, each component of the base editing system can be introduced to the target cell individually, or in combination. For instance, a fusion protein may be packaged into nanoparticle such as liposome. In another example, a guide RNA and a protein may be combined into a complex for introduction.

In some embodiments, some or all of the components of the base editing system can be introduced as one or more polynucleotides encoding them. These polynucleotides may be constructed as plasmids or viral vectors, without limitation.

In an example ex vivo approach, CD34+ hematopoietic stem and progenitor cells (HSPCs) can be collected from a patient by apheresis after mobilization with either filgrastim and plerixafor (in a patient with 0-thalassemia) or plerixafor alone (in a patient with SCD) after a minimum of 8 weeks of transfusions of packed red cells targeting a level of sickle hemoglobin of less than 30%. The HSPCs can then be edited with the disclosed gene editing technology, along with the designed sgRNA, to produce edited cells. DNA sequencing can be used to evaluate the percentage of allelic editing at the on-target site.

Prior to infusion of the edited cells, the patient can be given a pharmacokinetically adjusted busulfan myeloablation. The edited cells can be administered through intravenous infusion.

### EXAMPLES

### Example 1. Base Editing at BCL11A/Gamma-Globin

This example tested a newly designed base editor, referred to as transformer Base Editor (tBE), which can specifically edit cytosines in target regions with no observable off-target mutations, to edit the BCL11A gene which is useful for treating 0-hemoglobinopathies.

The tBE fuses a base editor with a cytidine deaminase inhibitor to inhibit the activity of the cytidine deaminase until the tBE complex is assembled at the target genomic site. The tBE employs a sgRNA (about 20 nt) to bind at the target genomic site and a helper sgRNA (hsgRNA, 10-20 nt) to bind at a nearby region upstream to the target genomic site. The binding of two sgRNAs can guide the components of tBE to correctly assemble at the target genomic site for efficient base editing.

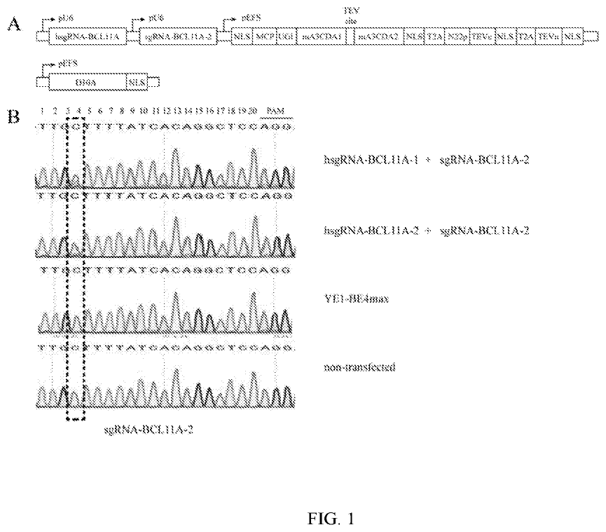

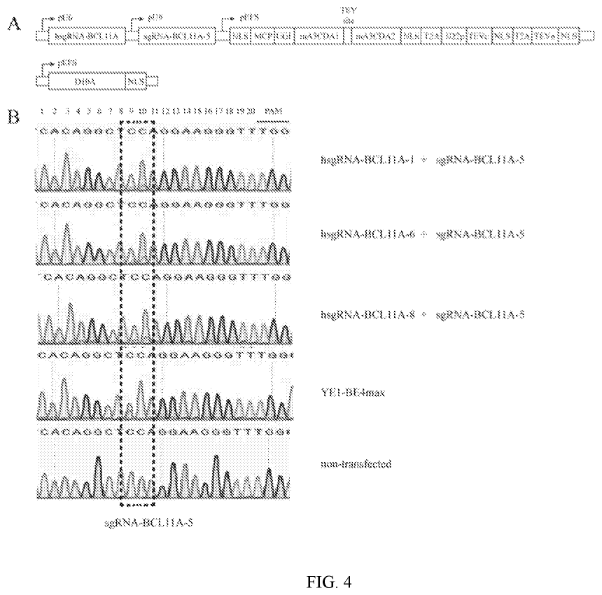

To test whether the tBE can perform high-specificity and high-efficiency base editing in BCL11A erythroid enhancer region, we designed 45 pairs (5 sgRNA×9 hsgRNA, as listed in Tables 1 and 2) of sgRNA/hsgRNAs to target the BCL11A erythroid enhancer region (the core BCL11A erythroid enhancer). For comparison, we co-transfected the sgRNAs in sgRNA/hsgRNA pairs with a previously reported BE, YE1-BE4max and a single sgRNA targeting the same genomic site with tBE (FIG. 1-4).

TABLE 1
sgRNA targeting BCL11A erythroid enhancer

| | | SEQ | | SEQ |
|---|---|---|---|---|
| | | ID | | ID |
| sgRNA | 10 nt | NO: | 20 nt | NO: |
| sgRNA-BCL11A-1 | cuuuuaucac | 1 | cuaacaguugcuuuuaucac | 2 |
| sgRNA-BCL11A-2 | cacaggcucc | 3 | uugcuuuuaucacaggcucc | 4 |
| sgRNA-BCL11A-3 | ggcuccagga | 5 | uuuuaucacaggcuccagga | 6 |
| sgRNA-BCL11A-4 | gcuccaggaa | 7 | uuuaucacaggcuccaggaa | 8 |
| sgRNA-BCL11A-5 | aggaaggguu | 9 | cacaggcuccaggaaggguu | 10 |

TABLE 2
hsgRNA targeting BCL11A erythroid enhancer

| | | SEQ | | SEQ |
|---|---|---|---|---|
| | | ID | | ID |
| hsgRNA | 10 nt | NO: | 20 nt | NO: |
| hsgRNA-BCL11A-1 | uaacacacca | 11 | cucuuagacauaacacacca | 12 |
| hsgRNA-BCL11A-2 | auaacacacc | 13 | acucuuagacauaacacacc | 14 |
| hsgRNA-BCL11A-3 | aauacaacuu | 15 | caccagggucaauacaacuu | 16 |
| hsgRNA-BCL11A-4 | acaacuuuga | 17 | cagggucaauacaacuuuga | 18 |
| hsgRNA-BCL11A-5 | cuuugaagcu | 19 | gucaauacaacuuugaagcu | 20 |
| hsgRNA-BCL11A-6 | aagcuagucu | 21 | uacaacuuugaagcuagucu | 22 |
| hsgRNA-BCL11A-7 | gcuagucuag | 23 | caacuuugaagcuagucuag | 24 |
| hsgRNA-BCL11A-8 | gucuagugca | 25 | uuugaagcuagucuagugca | 26 |
| hsgRNA-BCL11A-9 | gcaagcuaac | 27 | cuagucuagugcaagcuaac | 28 |

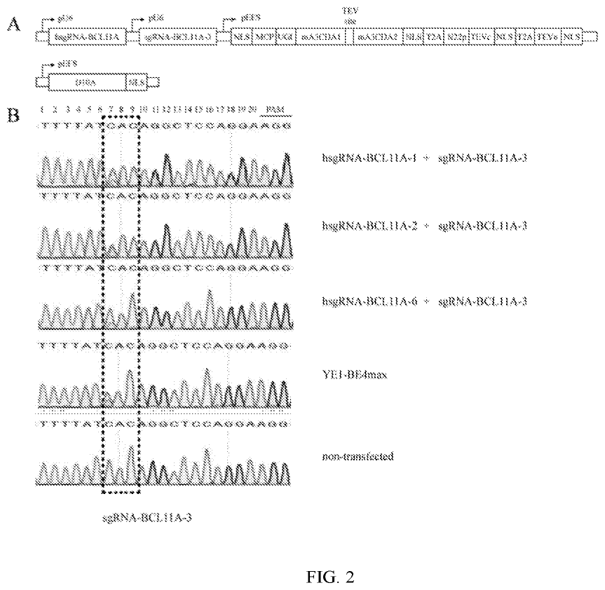

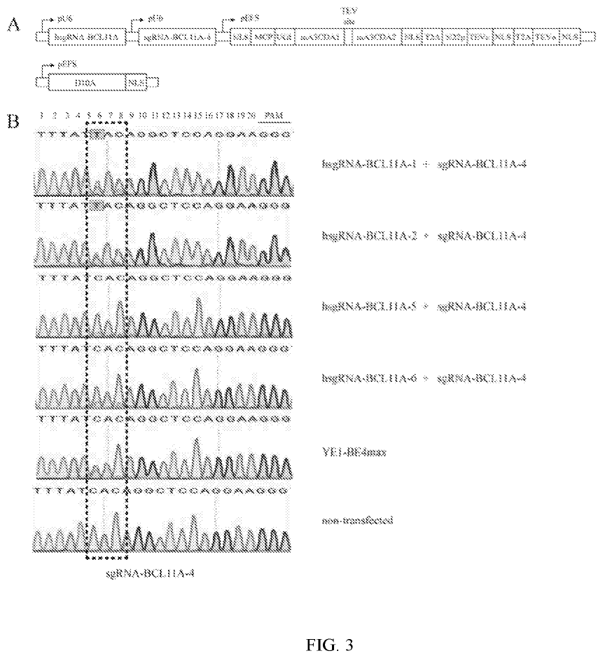

We extracted genomic DNA 72 hours after transfecting the plasmids into cells, and compared the C-to-T editing efficiencies of these BEs at target sites. From Sanger sequencing results, we found both tBE and YE1-BE4max induced gene editing in BCL11A erythroid enhancer region. It's worth noting that tBE induced similar or higher base editing efficiencies than YE1-BE4max at some target sites, such as the target sites for sgRNA-BCL11A-3/hsgRNA-BCL11A-2 (FIG. 2B) and sgRNA-BCL11A-4/hsgRNA-BCL11A-2 (FIG. 3B). These results indicate that tBE could perform highly efficient base editing in BCL11A erythroid enhancer region.

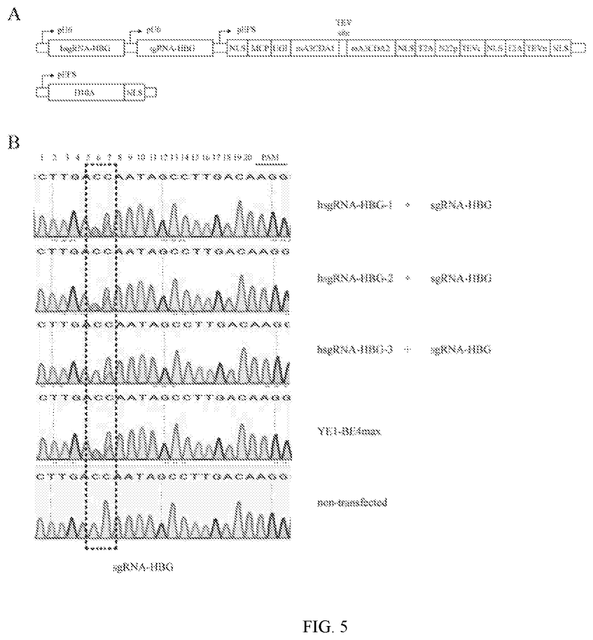

Next, we tested whether the tBE can perform high-specificity and high-efficiency base editing at the BCL11A binding motif in HBG1/2 promoters. We designed 3 pairs (1 sgRNA×3 hsgRNA, as listed in Tables 3 and 4) of sgRNA/hsgRNA to target the BCL11A binding motif in HBG1/2 promoters (FIG. 5). After 72 hours of plasmid transfection, we extracted genomic DNA from transfected cells. From the sanger sequencing results, we found both tBE and YE1-BE4max induced gene editing in the BCL11A binding motif of HBG1/2 promoters. Also, tBE induced similar or higher base editing efficiencies than YE1-BE4max at some target sites, such as the target sites for sgRNA-HBG/hsgRNA-HBG-1/2 (FIG. 5B).

TABLE 3
sgRNA targeting HBG1/2 promoters

| | | SEQ | | SEQ |
|---|---|---|---|---|
| | | ID | | ID |
| sgRNA | 10 nt | NO: | 20 nt | NO: |
| sgRNA-HBG | agccuugaca | 29 | cuugaccaauagccuugaca | 30 |

TABLE 4
hsgRNA targeting HBG1/2 promoters

| | | SEQ | | SEQ |
|---|---|---|---|---|
| | | ID | | ID |
| hsgRNA | 10 nt | NO: | 20 nt | NO: |
| hsgRNA-HBG-1 | acuccaccca | 31 | cccuggcuaaacuccaccca | 32 |
| hsgRNA-HBG-2 | cuccacccau | 33 | ccuggcuaaacuccacccau | 34 |
| hsgRNA-HBG-3 | acccaugggu | 35 | gcuaaacuccacccaugggu | 36 |

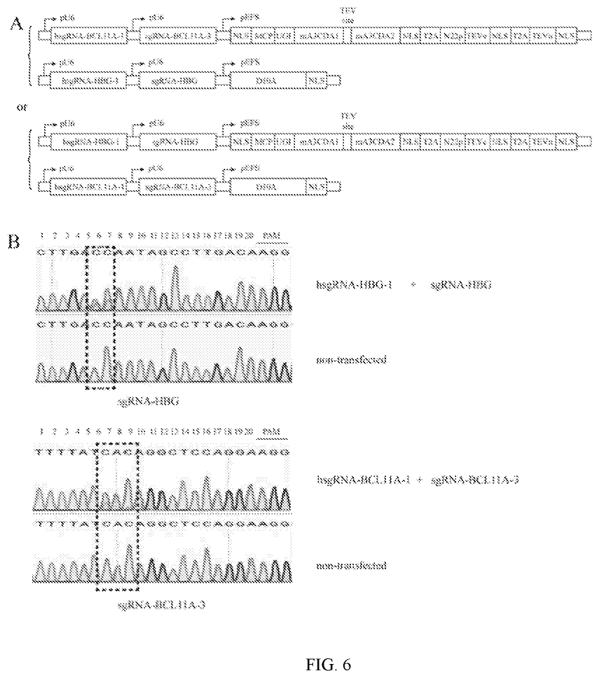

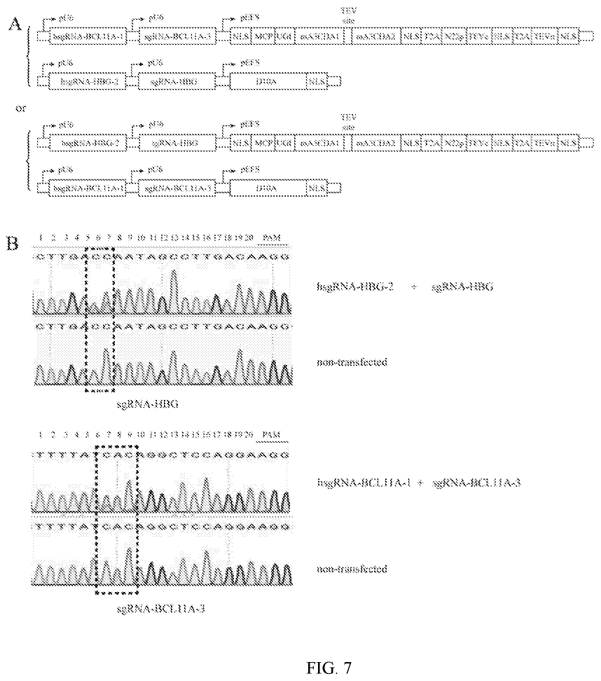

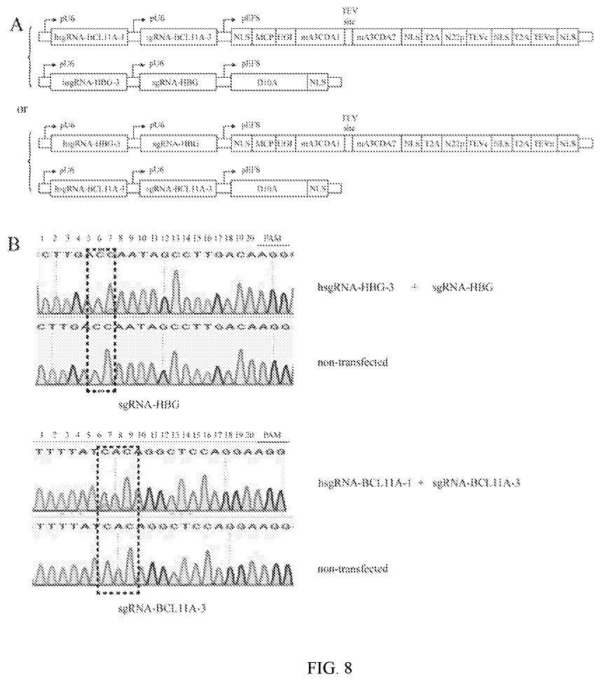

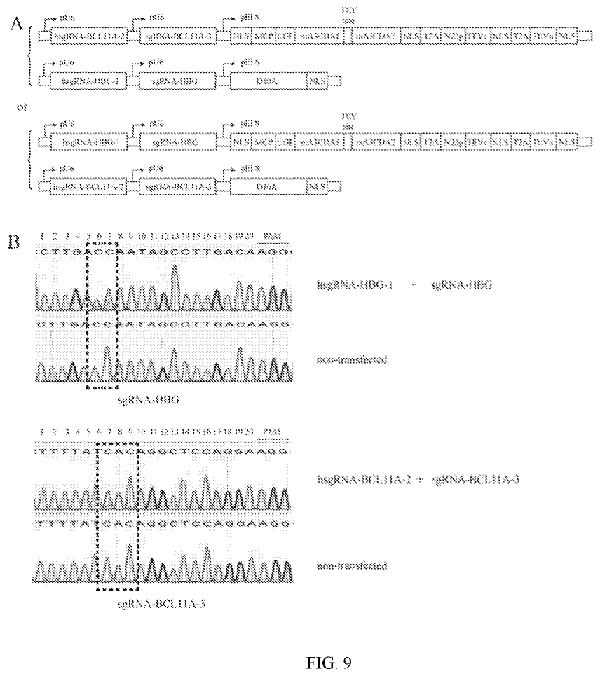

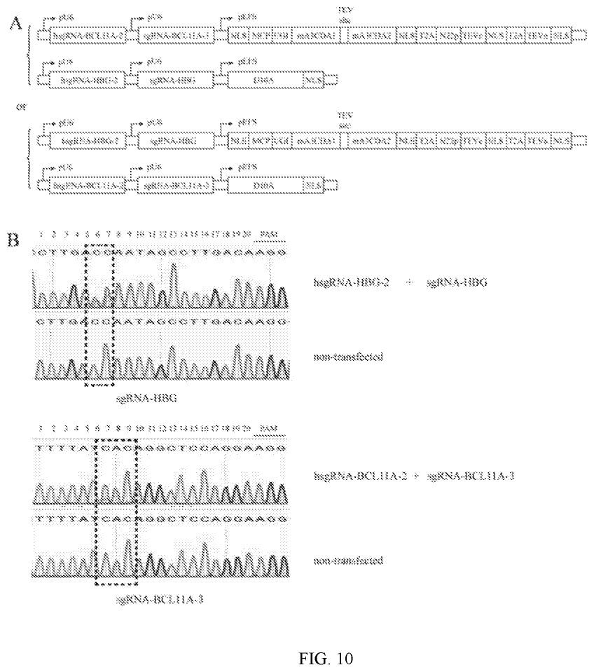

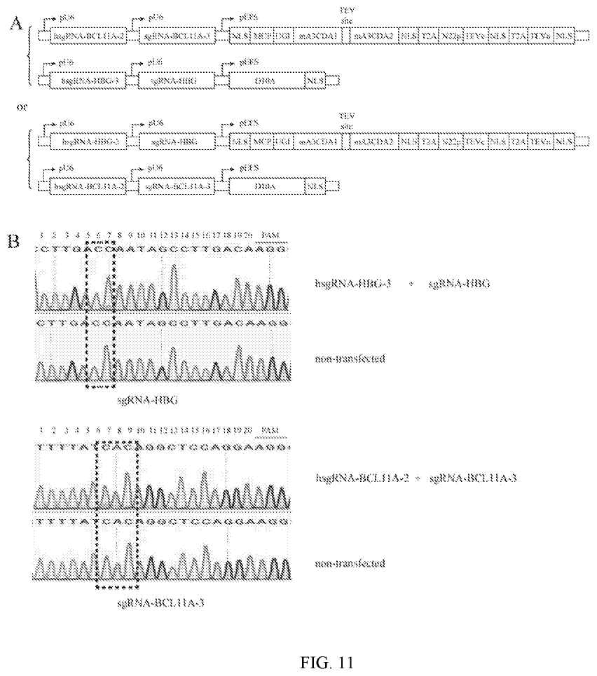

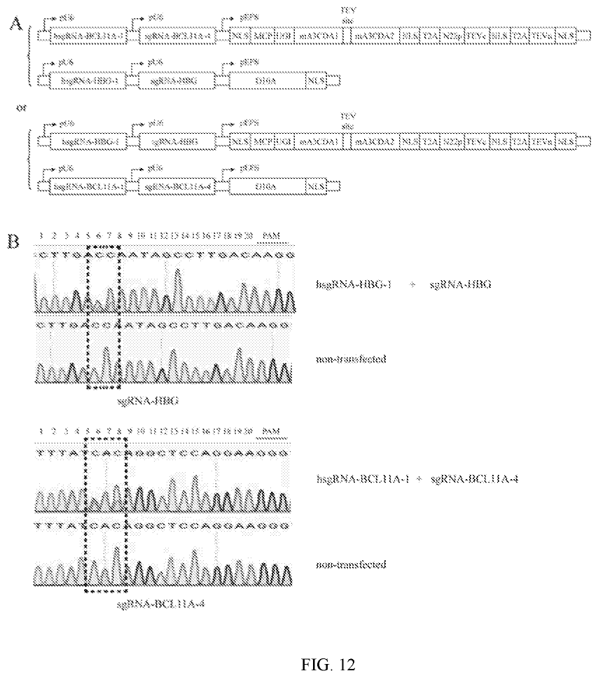

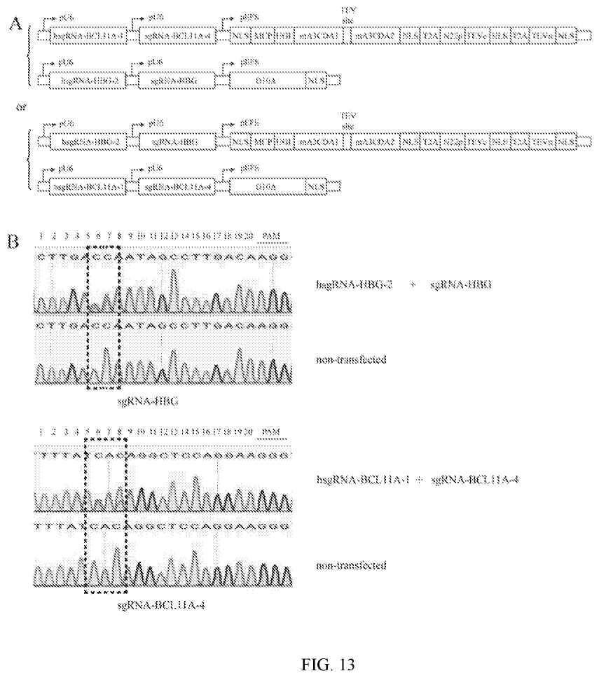

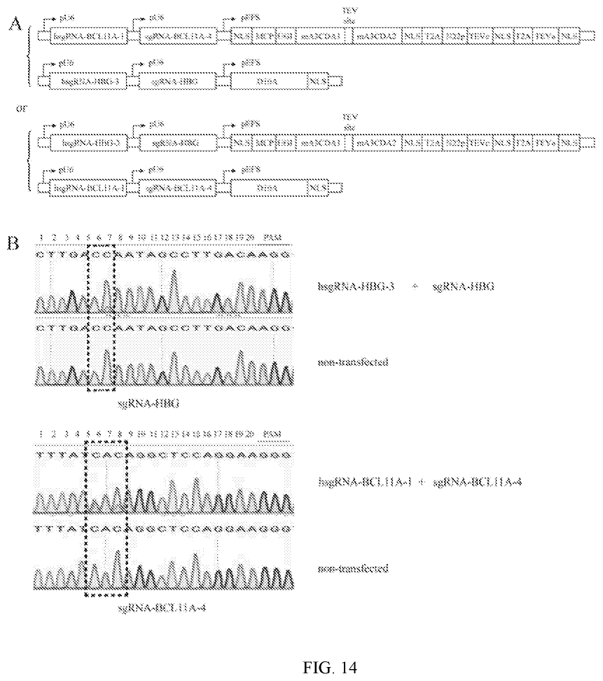

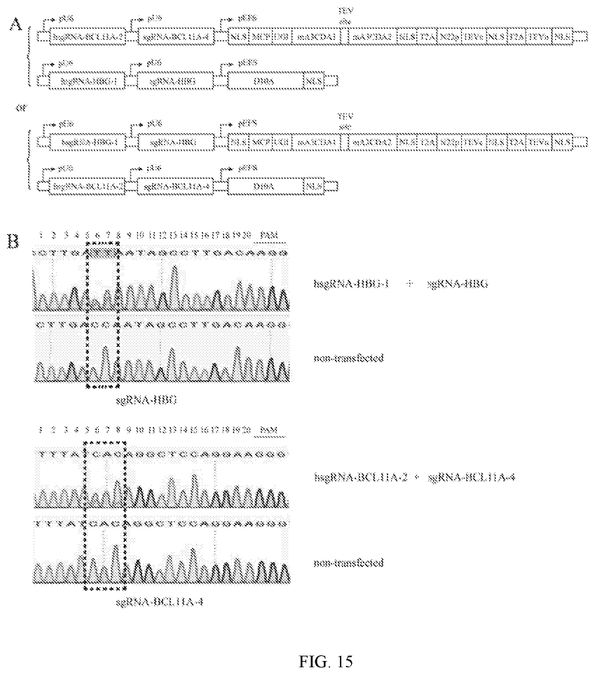

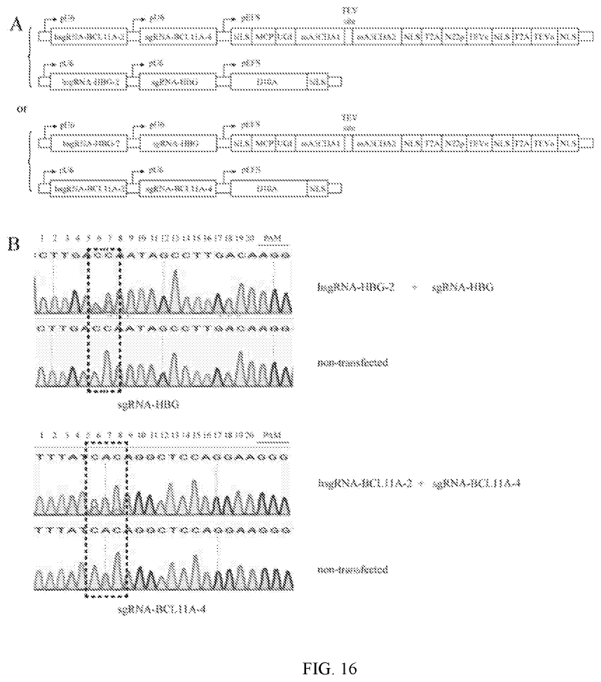

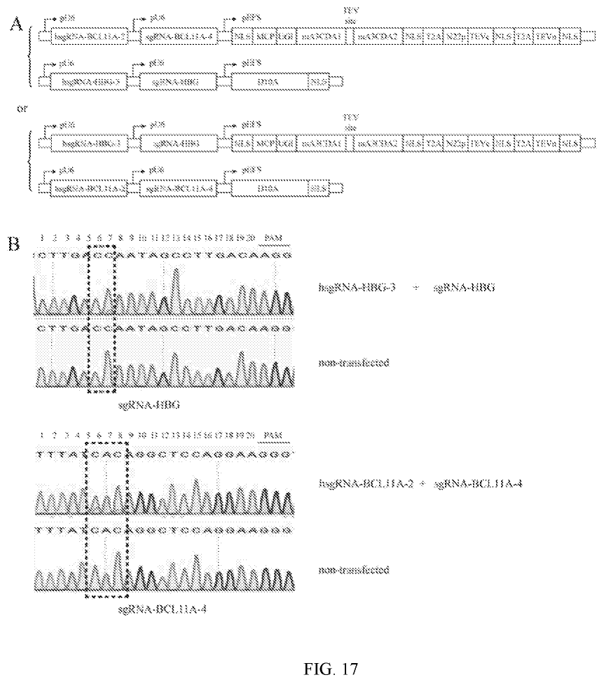

We then tested whether the tBE can perform high-specificity and high-efficiency at the BCL11A erythroid enhancer and HBG1/2 promoter regions simultaneously by modifying the plasmids of tBE system (FIG. 6A, 7A, 8A, 9A, 10A, 11A, 12A, 13A, 14A, 15A, 16A, 17A). Based on the experimental results obtained at these two sites, we chose the pairs of sgRNA/hsgRNA with relatively high editing efficiency (sgRNA-HBG/hsgRNA-HBG-1/2/3, sgRNA-BCL11A-3/hsgRNA-BCL11A-1/2 and sgRNA-BCL11A-4/hsgRNA-BCL11A-1/2) and transfected these plasmids in various combinations. After 72 hours of plasmid transfection, we extracted genomic DNA from transfected cells. From the sanger sequencing results, we found that the co-transfection of tBE with sgRNA-HBG/hsgRNA-HBG-2 and sgRNA-BCL11A-4/hsgRNA-BCL11A-1 induced the highest C-to-T mutation frequencies at both target sites (FIG. 13B).

This example used a highly precise and efficient base editing system (tBE) to perform base editing at the therapeutic genomic sites of the β-hemoglobinopathies. Furthermore, the tBE system, which contains Cas9 nickase (D10A), is less toxic than Cas9 nuclease as Cas9 nickase activates a lower level of p53 pathway than Cas9 nuclease. In addition, this example achieved high specificity and efficiency base editing individually or simultaneously at two therapeutic target sites, which can reactive a high expression level of γ-globin. This example therefore demonstrates a clinical use of tBE, especially in the gene therapies of the (3-hemoglobinopathies.

### Example 2. Base Editing at Other Sites Impacting Gamma-Globin Expression

The expression of BCL11A may be impacted by other cis elements or protein factors. There are three DNase I hypersensitive sites (DHSs), referred to as DHSs +62, +58, and +55 based on distance in kilobases from the transcription start site (TSS) of BCL11A. KLF1 is a key erythroid transcription factor that activates BCL11A directly by binding BCL11A's promoter. Furthermore, there is a GATA1-binding motif located in intron 4 of the NFIX gene, which could regulate the expression of BCL11A indirectly by influencing the expression of KLF1. In addition, ZBTB7A (zinc finger and BTB domain containing 7A), a repressor protein, could bind the HBG1/2 enhancer/promoter by identifying a conserved motif and repress the expression of HBG.

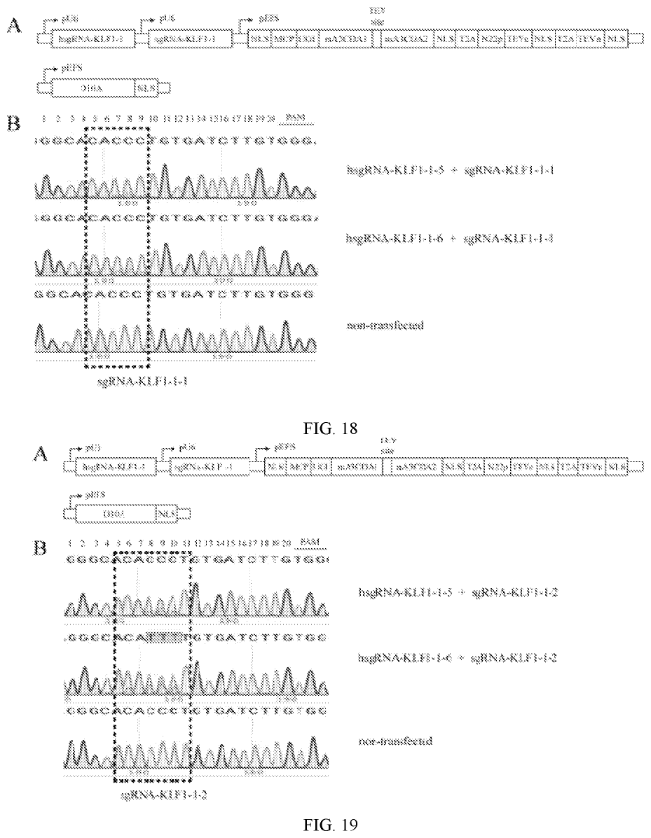

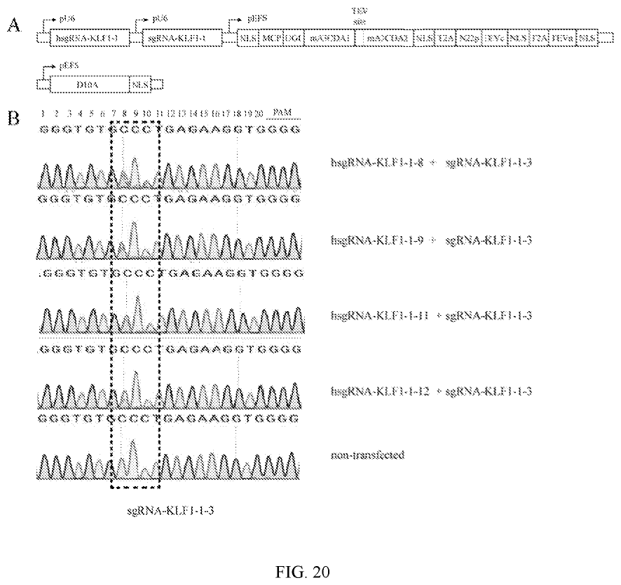

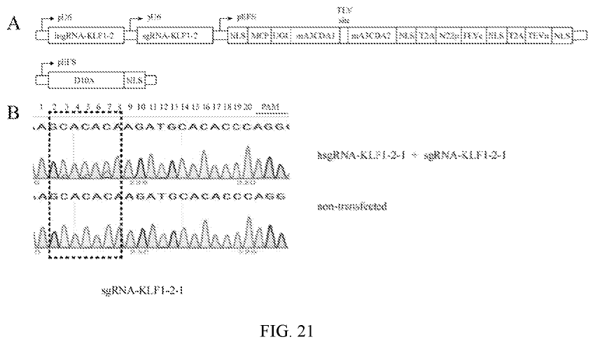

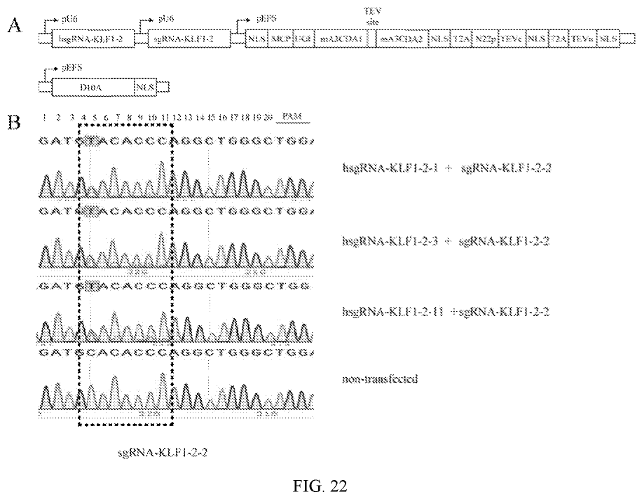

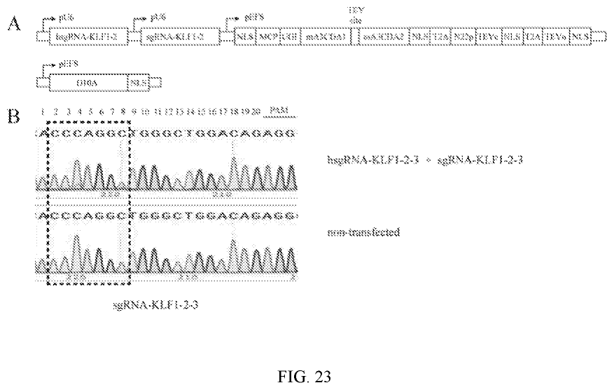

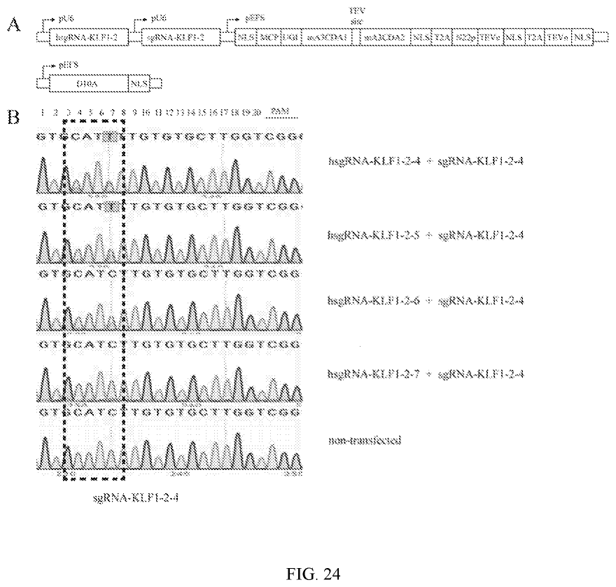

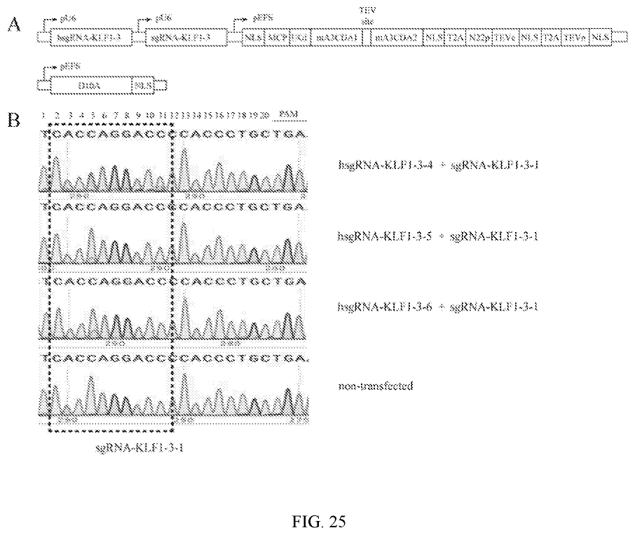

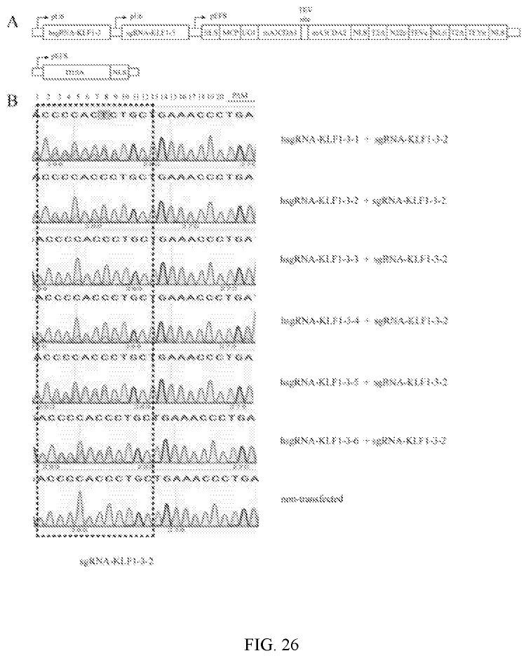

To test whether the tBE can perform high-specificity and high-efficiency base editing at the three KLF1 binding motifs of BCL11A (two core KLF1 binding motifs locate in +55 kb DHS of BCL11A erythroid enhancer region, the other one locates in 1 Mb upstream of BCL11A). We designed 50 pairs of sgRNA/hsgRNAs (Tables 5-7) to target the three KLF1 binding motifs (FIG. 18-26), and extracted genomic DNA 72 hours after transfecting them with tBE into cells. Sanger sequencing results (FIG. 18-26) demonstrate that the tBE, with the designed sgRNA/hsgRNA, efficiently induced gene editing at the three KLF1 binding motifs of BCL11A, which can be useful for activating the expression of the γ-globin gene. And, those bold marked hsgRNAs together with their corresponding sgRNAs worked efficiently at their target sites.

TABLE 5
sgRNA/hsgRNA targeting KLF1-binding motif 1 of BCL11A

| | | | | |
|---|---|---|---|---|
| | | SEQ | | SEQ |
| | | ID | | ID |
| sgRNA/hsgRNA | 10 nt | NO: | 20 nt | NO: |

Table 5.1 sgRNA-KLF1-1-1 and its hsgRNAs
targeting KLF1-binding motif 1 of BCL11A

| | | | | | |
|---|---|---|---|---|---|
| sgRNA | sgRNA-KLF1-1-1 | gugaucuugu | 37 | ggcacacccugugaucuugu | 38 |
| hsgRNA | hsgRNA-KLF1-1-4 | caccuucuca | 55 | gugagcuccccaccuucuca | 56 |
| | hsgRNA-KLF1-1-5 | caaugcuugg | 57 | caggaugaugcaaugcuugg | 58 |
| | hsgRNA-KLF1-1-6 | augcaaugcu | 59 | uaccaggaugaugcaaugcu | 60 |
| | hsgRNA-KLF1-1-7 | uccugguacc | 61 | cccauugccuuccugguacc | 62 |

Table 5.2 sgRNA-KLF1-1-2 and its sgRNAs
targeting KLF1-binding motif 1 of BCL11A

| | | | | | |
|---|---|---|---|---|---|
| sgRNA | sgRNA-KLF1-1-2 | ugugaucuug | 39 | gggcacacccugugaucuug | 40 |
| hsgRNA | hsgRNA-KLF1-1-4 | caccuucuca | 63 | gugagcuccccaccuucuca | 64 |
| | hsgRNA-KLF1-1-5 | caaugcuugg | 65 | caggaugaugcaaugcuugg | 66 |
| | hsgRNA-KLF1-1-6 | augcaaugcu | 67 | uaccaggaugaugcaaugcu | 68 |
| | hsgRNA-KLF1-1-7 | uccugguacc | 69 | cccauugccuuccugguacc | 70 |

Table 5.3 sgRNA-KLF1-1-3 and its hsgRNAs
targeting KLF1-binding motif 1 of BCLIIA

| | | | | | |
|---|---|---|---|---|---|
| sgRNA | sgRNA-KLF1-1-3 | ugagaaggug | 41 | gggugugcccugagaaggug | 42 |
| hsgRNA | hsgRNA-KLF1-1-8 | ggcuggacag | 71 | acccaggcugggcuggacag | 72 |
| | hsgRNA-KLF1-1-9 | caggcugggc | 73 | gaugcacacccaggcugggc | 74 |
| | hsgRNA-KLF1-1-10 | cacccaggcu | 75 | acaagaugcacacccaggcu | 76 |
| | hsgRNA-KLF1-1-11 | acacccaggc | 77 | cacaagaugcacacccaggc | 78 |
| | hsgRNA-KLF1-1-12 | augcacaccc | 79 | agcacacaagaugcacaccc | 80 |

TABLE 6
sgRNA/hsgRNA targeting KLF1-binding motif 2 of BCL11A

| | | | | |
|---|---|---|---|---|
| | | SEQ | | SEQ |
| | | ID | | ID |
| sgRNA/hsgRNA | 10 nt | NO: | 20 nt | NO: |

Table 6.1 sgRNA-KLF1-2-1 and its hsgRNAs
targeting KLF1-binding motif 2 of BCL11A

| | | | | | |
|---|---|---|---|---|---|
| sgRNA | sgRNA-KLF1-2-1 | augcacaccc | 43 | agcacacaagaugcacaccc | 44 |
| hsgRNA | hsgRNA-KLF1-2-1 | gaccgcucac | 81 | cagccuuggggaccgcucac | 82 |
| | hsgRNA-KLF1-2-2 | cacagccuug | 83 | gaaggcugggcacagccuug | 84 |
| | hsgRNA-KLF1-2-3 | cucccuaccg | 85 | gugccgacaacucccuaccg | 86 |
| | hsgRNA-KLF1-2-10 | gaccccuauc | 99 | ucccuaccgcgaccccuauc | 100 |
| | hsgRNA-KLF1-2-11 | ccccuaucag | 10 | ccuaccgcgaccccuaucag | 102 |
| | hsgRNA-KLF1-2-12 | cuaucagugc | 103 | accgcgaccccuaucagugc | 104 |

Table 6.2 sgRNA-KLF1-2-2 and its hsgRNAs
targeting KLF1-binding motif 2 of BCL11A

| | | | | | |
|---|---|---|---|---|---|
| sgRNA | sgRNA-KLF1-2-2 | caggcugggc | 45 | gaugcacacccaggcugggc | 46 |
| hsgRNA | hsgRNA-KLF1-2-1 | gaccgcucac | 81 | cagccuuggggaccgcucac | 82 |
| | hsgRNA-KLF1-2-2 | cacagccuug | 83 | gaaggcugggcacagccuug | 84 |
| | hsgRNA-KLF1-2-3 | cucccuaccg | 85 | gugccgacaacucccuaccg | 86 |
| | hsgRNA-KLF1-2-10 | gaccccuauc | 99 | ucccuaccgcgaccccuauc | 100 |
| | hsgRNA-KLF1-2-11 | ccccuaucag | 101 | ccuaccgcgaccccuaucag | 102 |
| | hsgRNA-KLF1-2-12 | cuaucagugc | 103 | accgcgaccccuaucagugc | 104 |

Table 6.3 sgRNA-KLF1-2-3 and its hsgRNAs
targeting KLF1-binding motif 2 of BCL11A

| | | | | | |
|---|---|---|---|---|---|
| sgRNA | sgRNA-KLF1-2-3 | ggcuggacag | 47 | acccaggcugggcuggacag | 48 |
| hsgRNA | hsgRNA-KLF1-2-1 | gaccgcucac | 81 | cagccuuggggaccgcucac | 82 |
| | hsgRNA-KLF1-2-2 | cacagccuug | 83 | gaaggcugggcacagccuug | 84 |
| | hsgRNA-KLF1-2-3 | cucccuaccg | 85 | gugccgacaacucccuaccg | 86 |
| | hsgRNA-KLF1-2-10 | gaccccuauc | 99 | ucccuaccgcgaccccuauc | 100 |
| | hsgRNA-KLF1-2-11 | ccccuaucag | 101 | ccuaccgcgaccccuaucag | 102 |
| | hsgRNA-KLF1-2-12 | cuaucagugc | 103 | accgcgaccccuaucagugc | 104 |

Table 6.4 sgRNA-KLF1-2-4 and its hsgRNAs
targeting KLF1-binding motif 2 of BCL11A

| | | | | | |
|---|---|---|---|---|---|
| sgRNA | sgRNA-KLF1-2-4 | ugugcuuggu | 49 | gugcaucuugugugcuuggu | 50 |
| hsgRNA | hsgRNA-KLF1-2-4 | gugaucuugu | 87 | ggcacacccugugaucuugu | 88 |
| | hsgRNA-KLF1-2-5 | ugugaucuug | 89 | gggcacacccugugaucuug | 90 |
| | hsgRNA-KLF1-2-6 | caccuucuca | 91 | gugagcuccccaccuucuca | 92 |
| | hsgRNA-KLF1-2-7 | caaugcuugg | 93 | caggaugaugcaaugcuugg | 94 |
| | hsgRNA-KLF1-2-8 | augcaaugcu | 95 | uaccaggaugaugcaaugcu | 96 |
| | hsgRNA-KLF1-2-9 | uccugguacc | 97 | cccauugccuuccugguacc | 98 |

TABLE 7
sgRNA/hsgRNA targeting KLF1-binding motif 3 of BCL11A

| | | | | |
|---|---|---|---|---|
| | | SEQ | | SEQ |
| | | ID | | ID |
| sgRNA/hsgRNA | 10 nt | NO: | 20 nt | NO: |

Table 7.1 sgRNA-KLF1-3-1 and its hsgRNAs
targeting KLF1-binding motif 3 of BCLIIA

| | | | | | |
|---|---|---|---|---|---|
| sgRNA | sgRNA-KLF1-3-1 | cccacccugc | 51 | ucaccaggaccccacccugc | 52 |
| hsgRNA | hsgRNA-KLF1-3-1 | cagccaggac | 105 | ggccaucugccagccaggac | 106 |
| | hsgRNA-KLF1-3-2 | ucugccagcc | 107 | aagguggccaucugccagcc | 108 |
| | hsgRNA-KLF1-3-3 | agcaagaagg | 109 | gugaaaacgcagcaagaagg | 110 |
| | hsgRNA-KLF1-3-4 | cgcagcaaga | 111 | caugugaaaacgcagcaaga | 112 |
| | hsgRNA-KLF1-3-5 | aaacgcagca | 113 | ugccaugugaaaacgcagca | 114 |
| | hsgRNA-KLF1-3-6 | gugaaaacgc | 115 | ucucugccaugugaaaacgc | 116 |

Table 7.2 sgRNA-KLF1-3-2 and its hsgRNAs
targeting KLF1-binding motif 3 of BCL11A

| | | | | | |
|---|---|---|---|---|---|
| sgRNA | sgRNA-KLF1-3-2 | gcugaaaccc | 53 | accccacccugcugaaaccc | 54 |
| hsgRNA | hsgRNA-KLF1-3-1 | cagccaggac | 105 | ggccaucugccagccaggac | 106 |
| | hsgRNA-KLF1-3-2 | ucugccagcc | 107 | aagguggccaucugccagcc | 108 |
| | hsgRNA-KLF1-3-3 | agcaagaagg | 109 | gugaaaacgcagcaagaagg | 110 |
| | hsgRNA-KLF1-3-4 | cgcagcaaga | 111 | caugugaaaacgcagcaaga | 112 |
| | hsgRNA-KLF1-3-5 | aaacgcagca | 113 | ugccaugugaaaacgcagca | 114 |
| | hsgRNA-KLF1-3-6 | gugaaaacgc | 115 | ucucugccaugugaaaacgc | 116 |

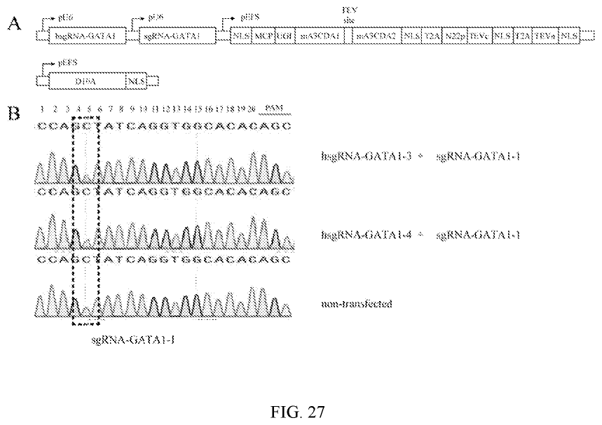

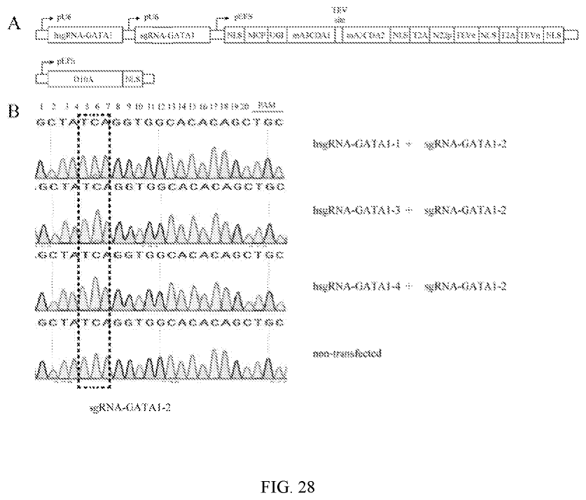

We also tested whether the tBE can perform base editing at the GATA1-binding motif located in intron 4 of the NFIX gene. We designed more than 20 pairs of sgRNA/hsgRNAs to target the GATA1-binding motif (FIG. 27-28) and extracted genomic DNA 72 hours after transfecting them with tBE into cells. Sanger sequencing results (FIG. 27-28) demonstrate that the tBE, with the designed sgRNA/hsgRNA, efficiently induced gene editing at the GATA1-binding motif located in the NFIX gene, which can be useful for activating the expression of the γ-globin gene.

TABLE 8
sgRNA/hsgRNA targeting GATA1-binding motif of NFIX

| | | | | |
|---|---|---|---|---|
| | | SEQ | | SEQ |
| | | ID | | ID |
| sgRNA/hsgRNA | 10 nt | NO: | 20 nt | NO: |

Table 8.1 sgRNA-GATA-1 and its hsgRNAs
targeting GATA1-binding motif of NFIX

| | | | | | |
|---|---|---|---|---|---|
| sgRNA | sgRNA-GATA-1 | gguggcacac | 117 | ccagcuaucagguggcacac | 118 |
| hsgRNA | hsgRNA-GATA-1 | cacagcuggu | 123 | gacagcugugcacagcuggu | 124 |
| | hsgRNA-GATA-2 | ugugcacagc | 125 | uggggacagcugugcacagc | 126 |
| | hsgRNA-GATA-3 | ugcggccaug | 127 | ggaggcacugugcggccaug | 128 |
| | hsgRNA-GATA-4 | ugugcggcca | 129 | ggggaggcacugugcggcca | 130 |
| | hsgRNA-GATA-5 | ggaacagcug | 131 | ugcaggacagggaacagcug | 132 |
| | hsgRNA-GATA-6 | agggaacagc | 133 | gcugcaggacagggaacagc | 134 |
| | hsgRNA-GATA-7 | gcugcaggac | 135 | aagcagcccagcugcaggac | 136 |
| | hsgRNA-GATA-8 | gcccagcugc | 137 | caauuaagcagcccagcugc | 138 |

Table 8.2 sgRNA-GATA-2 and its hsgRNAs
targeting GATA1-binding motif of NFIX

| | | | | | |
|---|---|---|---|---|---|
| sgRNA | sgRNA-GATA-2 | ggcacacagc | 119 | gcuaucagguggcacacagc | 120 |
| hsgRNA | hsgRNA-GATA-1 | cacagcuggu | 123 | gacagcugugcacagcuggu | 124 |
| | hsgRNA-GATA-2 | ugugcacagc | 125 | uggggacagcugugcacagc | 126 |
| | hsgRNA-GATA-3 | ugcggccaug | 127 | ggaggcacugugcggccaug | 128 |
| | hsgRNA-GATA-4 | ugugcggcca | 129 | ggggaggcacugugcggcca | 130 |
| | hsgRNA-GATA-5 | ggaacagcug | 131 | ugcaggacagggaacagcug | 132 |
| | hsgRNA-GATA-6 | agggaacagc | 133 | gcugcaggacagggaacagc | 134 |
| | hsgRNA-GATA-7 | gcugcaggac | 135 | aagcagcccagcugcaggac | 136 |
| | hsgRNA-GATA-8 | gcccagcugc | 137 | caauuaagcagcccagcugc | 138 |

Table 8.3 sgRNA-GATA-3 and its hsgRNAs
targeting GATA1-binding motif of NFIX

| | | | | | |
|---|---|---|---|---|---|
| sgRNA | sgRNA-GATA-3 | acacagcugc | 121 | aucagguggcacacagcugc | 122 |
| hsgRNA | hsgRNA-GATA-1 | cacagcuggu | 123 | gacagcugugcacagcuggu | 124 |
| | hsgRNA-GATA-2 | ugugcacagc | 125 | uggggacagcugugcacagc | 126 |
| | hsgRNA-GATA-3 | ugcggccaug | 127 | ggaggcacugugcggccaug | 128 |
| | hsgRNA-GATA-4 | ugugcggcca | 129 | ggggaggcacugugcggcca | 130 |
| | hsgRNA-GATA-5 | ggaacagcug | 131 | ugcaggacagggaacagcug | 132 |
| | hsgRNA-GATA-6 | agggaacagc | 133 | gcugcaggacagggaacagc | 134 |
| | hsgRNA-GATA-7 | gcugcaggac | 135 | aagcagcccagcugcaggac | 136 |
| | hsgRNA-GATA-8 | gcccagcugc | 137 | caauuaagcagcccagcugc | 138 |

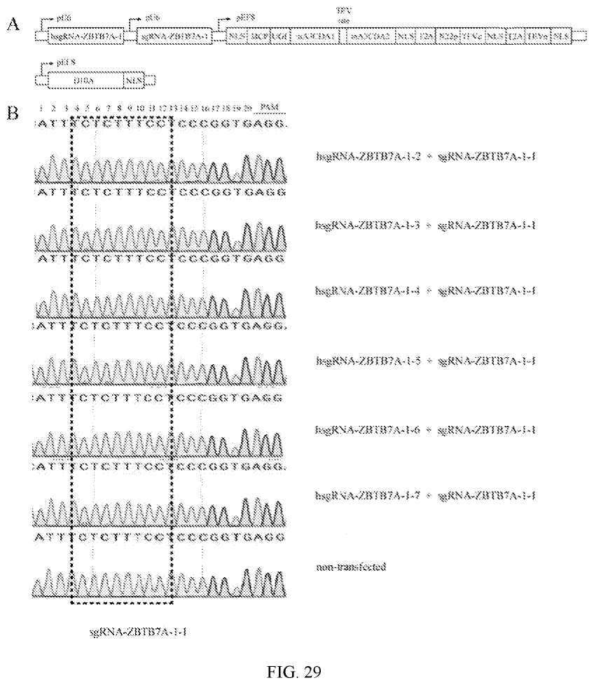

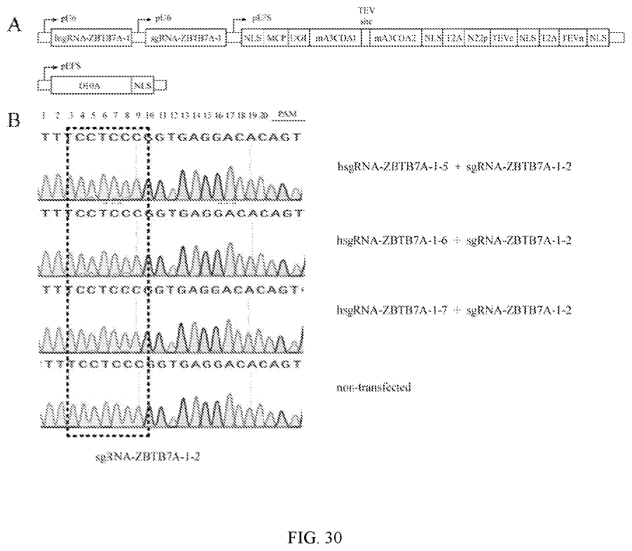

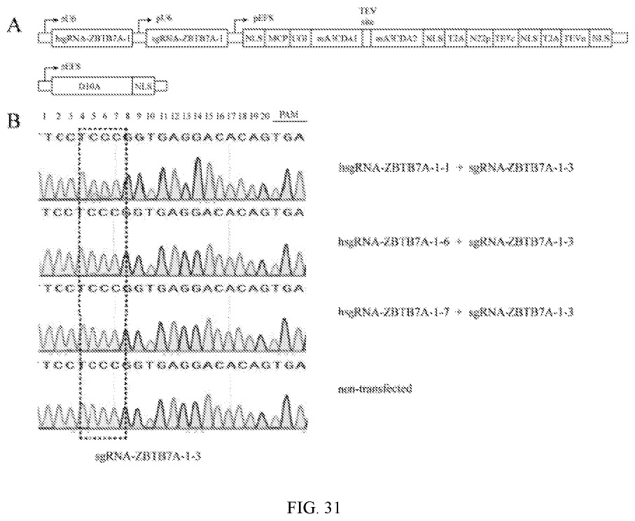

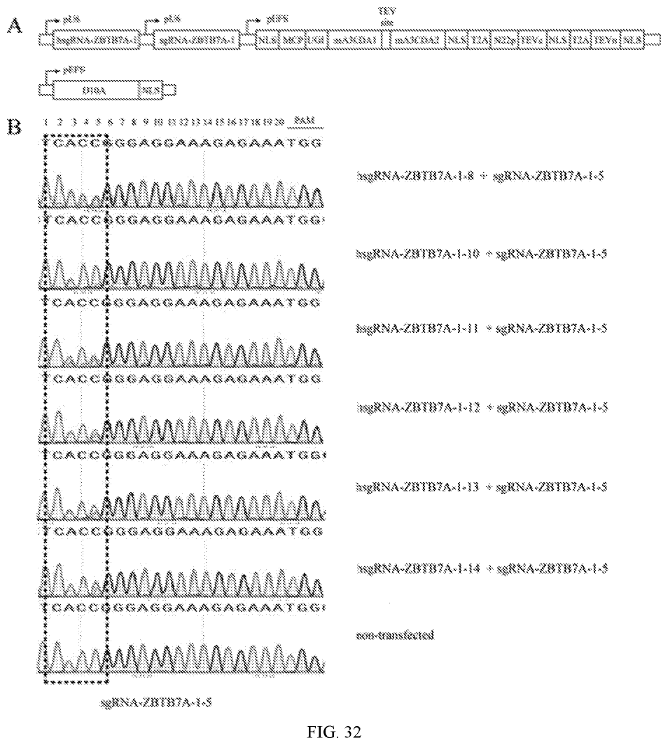

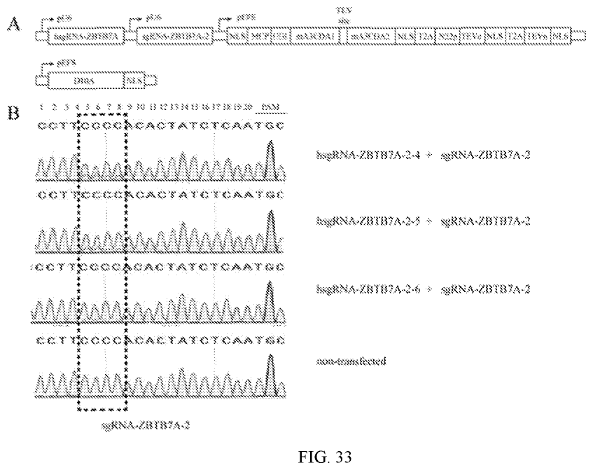

In addition, we tested whether the tBE can perform base editing at the two ZBTB7A-binding motifs located in the HBG1/2 promoter/enhancer. We designed 41 pairs of sgRNA/hsgRNAs to target the ZBTB7A-binding motifs (FIG. 29-33) and extracted genomic DNA 72 hours after transfecting them with tBE into cells. Sanger sequencing results (FIG. 29-33) demonstrate that the tBE, with the designed sgRNA/hsgRNA, efficiently induced gene editing at the two ZBTB7A-binding motifs located in HBG1/2 promoter/enhancer, which can be useful for activating the expression of the γ-globin gene.

TABLE 9
sgRNA/hsgRNA targeting ZBTB7A-binding motif 1 of HBG1/2

| | | | | |
|---|---|---|---|---|
| | | SEQ | | SEQ |
| | | ID | | ID |
| sgRNA/hsgRNA | 10 nt | NO: | 20 nt | NO: |

Table 9.1 sgRNA-ZBTB7A-1-1 and its hsgRNAs
targeting ZBTB7A-binding motif 1 of HBG1/2

| | | | | | |
|---|---|---|---|---|---|
| sgRNA | sgRNA-ZBTB7A-1-1 | ccucccggug | 139 | auuucucuuuccucccggug | 140 |
| hsgRNA | hsgRNA-ZBTB7A-1-1 | agaagcagag | 151 | uuuccuuaucagaagcagag | 152 |
| | hsgRNA-ZBTB7A-1-2 | uuuccuuauc | 153 | auaaaauuauuuuccuuauc | 154 |
| | hsgRNA-ZBTB7A-1-3 | ucauaagagc | 155 | uuagaugagcucauaagagc | 156 |
| | hsgRNA-ZBTB7A-1-4 | aaaaguaauu | 157 | gaggcuuuugaaaaguaauu | 158 |
| | hsgRNA-ZBTB7A-1-5 | ucuguggggg | 159 | acugaccuuaucuguggggg | 160 |
| | hsgRNA-ZBTB7A-1-6 | uuaucugugg | 161 | ccaacugaccuuaucugugg | 162 |
| | hsgRNA-ZBTB7A-1-7 | accuuaucug | 163 | cucccaacugaccuuaucug | 164 |

Table 9.2 sgRNA-ZBTB7A-1-2 and its hsgRNAs
targeting ZBTB7A-binding motif 1 of HBG1/2

| | | | | | |
|---|---|---|---|---|---|
| sgRNA | sgRNA-ZBTB7A-1-2 | gugaggacac | 141 | uuuccucccggugaggacac | 142 |
| hsgRNA | hsgRNA-ZBTB7A-1-1 | agaagcagag | 151 | uuuccuuaucagaagcagag | 152 |
| | hsgRNA-ZBTB7A-1-2 | uuuccuuauc | 153 | auaaaauuauuuuccuuauc | 154 |
| | hsgRNA-ZBTB7A-1-3 | ucauaagagc | 155 | uuagaugagcucauaagagc | 156 |
| | hsgRNA-ZBTB7A-1-4 | aaaaguaauu | 157 | gaggcuuuugaaaaguaauu | 158 |
| | hsgRNA-ZBTB7A-1-5 | ucuguggggg | 159 | acugaccuuaucuguggggg | 160 |
| | hsgRNA-ZBTB7A-1-6 | uuaucugugg | 161 | ccaacugaccuuaucugugg | 162 |
| | hsgRNA-ZBTB7A-1-7 | accuuaucug | 163 | cucccaacugaccuuaucug | 164 |

Table 9.3 sgRNA-ZBTB7A-1-3 and its hsgRNAs
targeting ZBTB7A-binding motif 1 of HBG1/2

| | | | | | |
|---|---|---|---|---|---|
| sgRNA | sgRNA-ZBTB7A-1-3 | gaggacacag | 143 | uccucccggugaggacacag | 144 |
| hsgRNA | hsgRNA-ZBTB7A-1-1 | agaagcagag | 151 | uuuccuuaucagaagcagag | 152 |
| | hsgRNA-ZBTB7A-1-2 | uuuccuuauc | 153 | auaaaauuauuuuccuuauc | 154 |
| | hsgRNA-ZBTB7A-1-3 | ucauaagagc | 155 | uuagaugagcucauaagagc | 156 |
| | hsgRNA-ZBTB7A-1-4 | aaaaguaauu | 157 | gaggcuuuugaaaaguaauu | 158 |
| | hsgRNA-ZBTB7A-1-5 | ucuguggggg | 159 | acugaccuuaucuguggggg | 160 |
| | hsgRNA-ZBTB7A-1-6 | uuaucugugg | 161 | ccaacugaccuuaucugugg | 162 |
| | hsgRNA-ZBTB7A-1-7 | accuuaucug | 163 | cucccaacugaccuuaucug | 164 |

Table 9.4 sgRNA-ZBTB7A-1-4 and its hsgRNAs
targeting ZBTB7A-binding motif 1 of HBG1/2

| | | | | | |
|---|---|---|---|---|---|
| sgRNA | sgRNA-ZBTB7A-1-4 | ggacacagug | 145 | cucccggugaggacacagug | 146 |
| hsgRNA | hsgRNA-ZBTB7A-1-1 | agaagcagag | 151 | uuuccuuaucagaagcagag | 152 |
| | hsgRNA-ZBTB7A-1-2 | uuuccuuauc | 153 | auaaaauuauuuuccuuauc | 154 |
| | hsgRNA-ZBTB7A-1-3 | ucauaagagc | 155 | uuagaugagcucauaagagc | 156 |
| | hsgRNA-ZBTB7A-1-4 | aaaaguaauu | 157 | gaggcuuuugaaaaguaauu | 158 |
| | hsgRNA-ZBTB7A-1-5 | ucuguggggg | 159 | acugaccuuaucuguggggg | 160 |
| | hsgRNA-ZBTB7A-1-6 | uuaucugugg | 161 | ccaacugaccuuaucugugg | 162 |
| | hsgRNA-ZBTB7A-1-7 | accuuaucug | 163 | cucccaacugaccuuaucug | 164 |

Table 9.5 sgRNA-ZBTB7A-1-5 and its hsgRNAs
targeting ZBTB7A-binding motif 1 of HBG1/2

| | | | | | |
|---|---|---|---|---|---|
| sgRNA | sgRNA-ZBTB7A-1-5 | gaaagagaaa | 147 | ucaccgggaggaaagagaaa | 148 |
| hsgRNA | hsgRNA-ZBTB7A-1-8 | uugcagaugg | 165 | ucuuccuggauugcagaugg | 166 |
| | hsgRNA-ZBTB7A-1-9 | ggauugcaga | 167 | uucucuuccuggauugcaga | 168 |
| | hsgRNA-ZBTB7A-1-10 | guucucuucc | 169 | cguggucaggguucucuucc | 170 |
| | hsgRNA-ZBTB7A-1-11 | cucgugguca | 171 | ugaaggcugacucgugguca | 172 |
| | hsgRNA-ZBTB7A-1-12 | acucgugguc | 173 | cugaaggcugacucgugguc | 174 |
| | hsgRNA-ZBTB7A-1-13 | ggcugacucg | 175 | cauuucugaaggcugacucg | 176 |
| | hsgRNA-ZBTB7A-1-14 | acauuucuga | 177 | guuuuuucucacauuucuga | 178 |

TABLE 10
sgRNA/hsgRNA targeting ZBTB7A-binding motif 2 of HBG1/2

| | | | SEQ | | SEQ |
|---|---|---|---|---|---|
| | | | ID | | ID |
| | sgRNA/hsgRNA | 10 nt | NO: | 20 nt | NO: |
| sgRNA | sgRNA-ZBTB7A-2 | acuaucucaa | 149 | ccuuccccacacuaucucaa | 150 |
| hsgRNA | hsgRNA-ZBTB7A-2-1 | auccucuugg | 179 | aauuagcaguauccucuugg | 180 |
| | hsgRNA-ZBTB7A-2-2 | aguauccucu | 181 | aaaaauuagcaguauccucu | 182 |
| | hsgRNA-ZBTB7A-2-3 | aaaaaaaauu | 183 | caaaggcuauaaaaaaaauu | 184 |
| | hsgRNA-ZBTB7A-2-4 | aacaaggcaa | 185 | acugaaucggaacaaggcaa | 186 |
| | hsgRNA-ZBTB7A-2-5 | aaucggaaca | 187 | ggaaugacugaaucggaaca | 188 |
| | hsgRNA-ZBTB7A-2-6 | augacugaau | 189 | aaaaacuggaaugacugaau | 190 |

TABLE 11
sgRNA/hsgRNA targeting the zinc finger structure of BCL11A

| | | | | |
|---|---|---|---|---|
| | | SEQ | | SEQ |
| | | ID | | ID |
| sgRNA/hsgRNA | 10 nt | NO: | 20 nt | NO: |

Table 11.1 sgRNA-T7431-1 and its
hsgRNAs targeting T743 of BCL11A

| | | | | | |
|---|---|---|---|---|---|
| sgRNA | sgRNA-T7431-1 | gugaguacug | 353 | agcgacacuugugaguacug | 354 |
| hsgRNA | hsgRNA-T7431-1-1 | gggcccgggc | 431 | uuagugguccgggcccgggc | 432 |
| | hsgRNA-T7431-1-3 | gguccgggcc | 433 | ccauauuagugguccgggcc | 434 |
| | hsgRNA-T7431-1-5 | auuagugguc | 435 | cacgccccauauuagugguc | 436 |

Table 11.2 sgRNA-T7431-2 and its
hsgRNAs targeting T743 of BCLIIA

| | | | | | |
|---|---|---|---|---|---|
| sgRNA | sgRNA-T7431-2 | ugaguacugu | 355 | gcgacacuugugaguacugu | 356 |
| hsgRNA | hsgRNA-T7431-2-1 | gggcccgggc | 437 | uuagugguccgggcccgggc | 438 |
| | hsgRNA-T7431-2-3 | gguccgggcc | 439 | ccauauuagugguccgggcc | 440 |
| | hsgRNA-T7431-2-5 | auuagugguc | 441 | cacgccccauauuagugguc | 442 |

Table 11.3 sgRNA-C747Y-G748K/R/E and its
hsgRNAs targeting C747 and G748 of BCL11A

| | | | | | |
|---|---|---|---|---|---|
| sgRNA | sgRNA-C747Y- | uacucacaag | 357 | uuucccacaguacucacaag | 358 |
| | G748K/R/E | | | | |
| hsgRNA | hsgRNA-C747Y- | cuguggacag | 443 | guggcuucuccuguggacag | 444 |
| | G748K/R/E-2 | | | | |
| | hsgRNA-C747Y- | uccuguggac | 445 | guguggcuucuccuguggac | 446 |
| | G748K/R/E-3 | | | | |
| | hsgRNA-C747Y- | cuucuccugu | 447 | gcccguguggcuucuccugu | 448 |
| | G748K/R/E-4 | | | | |

Table 11.4 sgRNA-S755N and its
hsgRNAs targeting S755 of BCL11A

| | | | | | |
|---|---|---|---|---|---|
| sgRNA | sgRNA-S755N | caguucuuga | 359 | gagauugcuacaguucuuga | 360 |
| hsgRNA | hsgRNA-S755N-1 | uucgcccgug | 449 | uauaaggccuuucgcccgug | 450 |
| | hsgRNA-S755N-2 | cuuucgcccg | 451 | uuuauaaggccuuucgcccg | 452 |
| | hsgRNA-S755N-3 | gccuuucgcc | 453 | cauuuauaaggccuuucgcc | 454 |

Table 11.5 sgRNA-L757F-1 and its
hsgRNAs targeting L757 of BCL11A

| | | | | | |
|---|---|---|---|---|---|
| sgRNA | sgRNA-L757F-1 | cacuguccac | 361 | guagcaaucucacuguccac | 362 |
| hsgRNA | hsgRNA-L757F-1-1 | ugaguacugu | 455 | gcgacacuugugaguacugu | 456 |
| | hsgRNA-L757F-1-2 | gugaguacug | 457 | agcgacacuugugaguacug | 458 |
| | hsgRNA-L757F-1-3 | gcucaaaaga | 459 | ggcaggcccagcucaaaaga | 460 |

Table 11.6 sgRNA-L757F-2 and its
hsgRNAs targeting L757 of BCL11A

| | | | | | |
|---|---|---|---|---|---|
| sgRNA | sgRNA-L757F-2 | uguccacagg | 363 | gcaaucucacuguccacagg | 364 |
| hsgRNA | hsgRNA-L757F-2-1 | ugaguacugu | 461 | gcgacacuugugaguacugu | 462 |
| | hsgRNA-L757F-2-2 | uugugaguac | 463 | gcagcgacacuugugaguac | 464 |
| | hsgRNA-L757F-2-3 | gacacuugug | 465 | cagacgcagcgacacuugug | 466 |

Table 11.7 sgRNA-L757F-T7581 and its
hsgRNAs targeting L757 and T758 of BCLIIA

| | | | | | |
|---|---|---|---|---|---|
| sgRNA | sgRNA-L757F-T7581 | ccacaggaga | 365 | aucucacuguccacaggaga | 366 |
| hsgRNA | hsgRNA-L757F-T7581-1 | acugugggaa | 467 | acuugugaguacugugggaa | 468 |
| | hsgRNA-L757F-T7581-2 | gacacuugug | 469 | cagacgcagcgacacuugug | 470 |
| | hsgRNA-L757F-T7581-3 | cagcgacacu | 471 | agggcagacgcagcgacacu | 472 |

Table 11.8 sgRNA-V7591 and its
hsgRNAs targeting V759 of BCL11A

| | | | | | |
|---|---|---|---|---|---|
| sgRNA | sgRNA-V7591 | agauugcuac | 367 | guggacagugagauugcuac | 368 |
| hsgRNA | hsgRNA-V7591-1 | gcauuuauaa | 473 | ugcacagcucgcauuuauaa | 474 |
| | hsgRNA-V7591-2 | cgcauuuaua | 475 | uugcacagcucgcauuuaua | 476 |

Table 11.9 sgRNA-H760Y and its
hsgRNAs targeting H760 of BCL11A

| | | | | | |
|---|---|---|---|---|---|
| sgRNA | sgRNA-H760Y | agaagccaca | 369 | uguccacaggagaagccaca | 370 |
| hsgRNA | hsgRNA-H760Y-1 | ugaguacugu | 477 | gcgacacuugugaguacugu | 478 |
| | hsgRNA-H760Y-2 | gugaguacug | 479 | agcgacacuugugaguacug | 480 |

Table 11.10 sgRNA-R761K and its
hsgRNAs targeting R761 of BCL11A

| | | | | | |
|---|---|---|---|---|---|
| sgRNA | sgRNA-R761K | acagugagau | 371 | ucuccuguggacagugagau | 372 |
| hsgRNA | hsgRNA-R761K-1 | uugcacagcu | 481 | acaggcauaguugcacagcu | 482 |
| | hsgRNA-R761K-2 | auaguugcac | 483 | gggcacaggcauaguugcac | 484 |

Table 11.11 sgRNA-R761K-R762K and its
hsgRNAs targeting R761 and R762 of BCL11A

| | | | | | |
|---|---|---|---|---|---|
| sgRNA | sgRNA-R761K-R762K | guggacagug | 373 | ggcuucuccuguggacagug | 374 |
| hsgRNA | hsgRNA-R761K-R762K-1 | uugcacagcu | 485 | acaggcauaguugcacagcu | 486 |
| | hsgRNA-R761K-R762K-2 | auaguugcac | 487 | gggcacaggcauaguugcac | 488 |

Table 11.12 sgRNA-R762K-S763N and its
hsgRNAs targeting R761 and S763 of BCL11A

| | | | | | |
|---|---|---|---|---|---|
| sgRNA | sgRNA-R762K-S763N | cuguggacag | 375 | guggcuucuccuguggacag | 376 |
| hsgRNA | hsgRNA-R762K-S763N-1 | uugcacagcu | 489 | acaggcauaguugcacagcu | 490 |
| | hsgRNA-R762K-S763N-2 | auaguugcac | 491 | gggcacaggcauaguugcac | 492 |

Table 11.13 sgRNA-H764Y and its
hsgRNAs targeting H764 of BCL11A

| | | | | | |
|---|---|---|---|---|---|
| sgRNA | sgRNA-H764Y | cacgggcgaa | 377 | ggagaagccacacgggcgaa | 378 |
| hsgRNA | hsgRNA-H764Y-1 | ugaguacugu | 493 | gcgacacuugugaguacugu | 494 |
| | hsgRNA-H764Y-2 | gugaguacug | 495 | agcgacacuugugaguacug | 496 |

Table 11.14 sgRNA-G766N/S/D and its
hsgRNAs targeting G766 of BCL11A

| | | | | | |
|---|---|---|---|---|---|
| sgRNA | sgRNA-G766N/S/D | gcuucuccug | 379 | cgcccguguggcuucuccug | 380 |
| | hsgRNA-G766N/S/D-1 | cucugggcac | 497 | gagcuugcuacucugggcac | 498 |
| hsgRNA | hsgRNA-G766N/S/D-2 | uugcuacucu | 499 | ccuggugagcuugcuacucu | 500 |
| | hsgRNA-G766N/S/D-3 | cuugcuacuc | 501 | gccuggugagcuugcuacuc | 502 |

Table 11.15 sgRNA-G766N/S/D-E767K and its
hsgRNAs targeting G766 and E767 of BCL11A

| | | | | | |
|---|---|---|---|---|---|
| sgRNA | sgRNA-G766N/S/D-E767K | uggcuucucc | 381 | uucgcccguguggcuucucc | 382 |
| hsgRNA | hsgRNA-G766N/S/D-E767K-1 | caggcauagu | 503 | acucugggcacaggcauagu | 504 |
| | hsgRNA-G766N/S/D-E767K-2 | gcacaggcau | 505 | gcuacucugggcacaggcau | 506 |

Table 11.16 sgRNA-R768K and its
hsgRNAs targeting R768 of BCL11A

| | | | | | |
|---|---|---|---|---|---|
| sgRNA | sgRNA-R768K | uucgcccgug | 383 | uauaaggccuuucgcccgug | 384 |
| hsgRNA | hsgRNA-R768K-1 | cucugggcac | 507 | gagcuugcuacucugggcac | 508 |
| | hsgRNA-R768K-2 | uugcuacucu | 509 | ccuggugagcuugcuacucu | 510 |
| | hsgRNA-R768K-3 | cuugcuacuc | 511 | gccuggugagcuugcuacuc | 512 |

Table 11.17 sgRNA-P769F/S/L and
its hsgRNAs targeting P769 of BCL11A

| | | | | | |
|---|---|---|---|---|---|
| sgRNA | sgRNA-P769F/S/L | aaaugcgagc | 385 | aaggccuuauaaaugcgagc | 386 |
| hsgRNA | hsgRNA-P769F/S/L-1 | gcaaucucac | 513 | aagaacuguagcaaucucac | 514 |
| | hsgRNA-P769F/S/L-2 | caagaacugu | 515 | ggaaagucuucaagaacugu | 516 |
| | hsgRNA-P769F/S/L-3 | cuucaagaac | 517 | gugggaaagucuucaagaac | 518 |

Table 11.18 sgRNA-C775Y and its
hsgRNAs targeting C775 of BCLIIA

| | | | | | |
|---|---|---|---|---|---|
| sgRNA | sgRNA-C775Y | cgcauuuaua | 387 | uugcacagcucgcauuuaua | 388 |
| hsgRNA | hsgRNA-C775Y-1 | uugcuacucu | 519 | ccuggugagcuugcuacucu | 520 |
| | hsgRNA-C775Y-2 | cuugcuacuc | 521 | gccuggugagcuugcuacuc | 522 |
| | hsgRNA-C775Y-3 | uucaugugcc | 523 | gccaugcguuuucaugugcc | 524 |

Table 11.19 sgRNA-A778V and its
hsgRNAs targeting A778 of BCL11A

| | | | | | |
|---|---|---|---|---|---|
| sgRNA | sgRNA-A778V | ugcccagagu | 389 | acuaugccugugcccagagu | 390 |
| hsgRNA | hsgRNA-A778V-1 | acgggcgaaa | 525 | gagaagccacacgggcgaaa | 526 |
| | hsgRNA-A778V-2 | cacgggcgaa | 527 | ggagaagccacacgggcgaa | 528 |
| | hsgRNA-A778V-3 | gccacacggg | 529 | cacaggagaagccacacggg | 530 |

Table 11.20 sgRNA-A778T and its
hsgRNAs targeting A778 of BCL11A

| | | | | | |
|---|---|---|---|---|---|
| sgRNA | sgRNA-A778T | uugcacagcu | 391 | acaggcauaguugcacagcu | 392 |
| hsgRNA | hsgRNA-A778T-1 | uucaugugcc | 531 | gccaugcguuuucaugugcc | 532 |
| | hsgRNA-A778T-2 | cguuuucaug | 533 | ccuggccaugcguuuucaug | 534 |
| | hsgRNA-A778T-3 | ugcguuuuca | 535 | caccuggccaugcguuuuca | 536 |

Table 11.21 sgRNA-A778V-A780V and its
hsgRNAs targeting A778 and A780 of BCL11A

| | | | | | |
|---|---|---|---|---|---|
| sgRNA | sgRNA-A778V-A780V | cagaguagca | 393 | ugccugugcccagaguagca | 394 |
| hsgRNA | hsgRNA-A778V-A780V-1 | acgggcgaaa | 537 | gagaagccacacgggcgaaa | 538 |
| | hsgRNA-A778V-A780V-2 | cacgggcgaa | 539 | ggagaagccacacgggcgaa | 540 |

Table 11.22 sgRNA-C779Y-A780T and its
hsgRNAs targeting C779 and A780 of BCL11A

| | | | | | |
|---|---|---|---|---|---|
| sgRNA | sgRNA-C779Y-A780T | auaguugcac | 395 | gggcacaggcauaguugcac | 396 |
| hsgRNA | hsgRNA-C779Y-A780T-1 | uucaugugcc | 541 | gccaugcguuuucaugugcc | 542 |
| | hsgRNA-C779Y-A780T-2 | cguuuucaug | 543 | ccuggccaugcguuuucaug | 544 |
| | hsgRNA-C779Y-A780T-3 | ugcguuuuca | 545 | caccuggccaugcguuuuca | 546 |

Table 11.23 sgRNA-Q781* and its
hsgRNAs targeting Q781 of BCLIIA

| | | | | | |
|---|---|---|---|---|---|
| sgRNA | sgRNA-Q781* | caagcucacc | 397 | cccagaguagcaagcucacc | 398 |
| hsgRNA | hsgRNA-Q781*-1 | cacgggcgaa | 547 | ggagaagccacacgggcgaa | 548 |
| | hsgRNA-Q781*-2 | gaagccacac | 549 | guccacaggagaagccacac | 550 |
| | hsgRNA-Q781*-3 | agaagccaca | 551 | uguccacaggagaagccaca | 552 |

Table 11.24 sgRNA-S782N and its
hsgRNAs targeting S782 of BCL11A

| | | | | | |
|---|---|---|---|---|---|
| sgRNA | sgRNA-S782N | gcacaggcau | 399 | gcuacucugggcacaggcau | 400 |
| hsgRNA | hsgRNA-S782N-1 | ccuggccaug | 553 | uccuuccccaccuggccaug | 554 |
| | hsgRNA-S782N-2 | caccuggcca | 555 | cguccuuccccaccuggcca | 556 |
| | hsgRNA-S782N-3 | uuccccaccu | 557 | guaaacguccuuccccaccu | 558 |

Table 11.25 sgRNA-S783N and its
hsgRNAs targeting S783 of BCL11A

| | | | | | |
|---|---|---|---|---|---|
| sgRNA | sgRNA-S783N | cucugggcac | 401 | gagcuugcuacucugggcac | 402 |
| hsgRNA | hsgRNA-S783N-1 | cuuccccacc | 559 | uguaaacguccuuccccacc | 560 |

Table 11.26 sgRNA-L785F and its
hsgRNAs targeting L785 of BCLIIA

| | | | | | |
|---|---|---|---|---|---|
| sgRNA | sgRNA-L785F | accaggcaca | 403 | uagcaagcucaccaggcaca | 404 |
| hsgRNA | hsgRNA-L785F-1 | aaaugcgagc | 561 | aaggccuuauaaaugcgagc | 562 |
| | hsgRNA-L785F-2 | uauaaaugcg | 563 | cgaaaggccuuauaaaugcg | 564 |
| | hsgRNA-L785F-3 | cuuauaaaug | 565 | ggcgaaaggccuuauaaaug | 566 |

Table 11.27 sgRNA-L785F-T786I and its
hsgRNAs targeting L785 and T786 of BCL11A

| | | | | | |
|---|---|---|---|---|---|
| sgRNA | sgRNA-L785F-T7861 | cacaugaaaa | 405 | gcucaccaggcacaugaaaa | 406 |
| hsgRNA | hsgRNA-L785F-T7861-1 | ugugcaacua | 567 | aaaugcgagcugugcaacua | 568 |
| | hsgRNA-L785F-T7861-2 | augcgagcug | 569 | ggccuuauaaaugcgagcug | 570 |
| | hsgRNA-L785F-T7861-3 | aaaugcgagc | 571 | aaggccuuauaaaugcgagc | 572 |

Table 11.28 sgRNA-R787K and its
hsgRNAs targeting S783 of BCL11A

| | | | | | |
|---|---|---|---|---|---|
| sgRNA | sgRNA-R787K | cuugcuacuc | 407 | gccuggugagcuugcuacuc | 408 |
| hsgRNA | hsgRNA-R787K-1 | cuuccccacc | 573 | uguaaacguccuuccccacc | 574 |

Table 11.29 sgRNA-T791M-H792Y-1 and its
hsgRNAs targeting T791 and H792 of BCL11A

| | | | | | |
|---|---|---|---|---|---|
| sgRNA | sgRNA-T791M-H792Y-1 | caggugggga | 409 | aacgcauggccaggugggga | 410 |
| hsgRNA | hsgRNA-T791M-H792Y-1-1 | ugcccagagu | 575 | acuaugccugugcccagagu | 576 |
| | hsgRNA-T791M-H792Y-1-2 | cugugcccag | 577 | gcaacuaugccugugcccag | 578 |
| | hsgRNA-T791M-H792Y-1-3 | gccugugccc | 579 | gugcaacuaugccugugccc | 580 |

Table 11.30 sgRNA-T791M-H792Y-2 and its
hsgRNAs targeting T791 and H792 of BCLIIA

| | | | | | |
|---|---|---|---|---|---|
| sgRNA | sgRNA-T791M-H792Y-2 | ggccaggugg | 411 | gaaaacgcauggccaggugg | 412 |
| hsgRNA | hsgRNA-T791M-H792Y-2-1 | ugcccagagu | 581 | acuaugccugugcccagagu | 582 |
| | hsgRNA-T791M-H792Y-2-2 | cugugcccag | 583 | gcaacuaugccugugcccag | 584 |
| | hsgRNA-T791M-H792Y-2-3 | gccugugccc | 585 | gugcaacuaugccugugccc | 586 |

Table 11.31 sgRNA-H792Y and its
hsgRNAs targeting H792 of BCL11A

| | | | | | |
|---|---|---|---|---|---|
| sgRNA | sgRNA-H792Y | agguggggaa | 413 | acgcauggccagguggggaa | 414 |
| hsgRNA | hsgRNA-H792Y-1 | ugcccagagu | 587 | acuaugccugugcccagagu | 588 |
| | hsgRNA-H792Y-2 | cugugcccag | 589 | gcaacuaugccugugcccag | 590 |
| | hsgRNA-H792Y-3 | gccugugccc | 591 | gugcaacuaugccugugccc | 592 |

Table 11.32 sgRNA-Q794* and its
hsgRNAs targeting Q794 of BCL11A

| | | | | | |
|---|---|---|---|---|---|
| sgRNA | sgRNA-Q794* | uggggaagga | 415 | cauggccagguggggaagga | 416 |
| hsgRNA | hsgRNA-Q794*-1 | cagaguagca | 593 | ugccugugcccagaguagca | 594 |
| | hsgRNA-Q794*-2 | ugcccagagu | 595 | acuaugccugugcccagagu | 596 |
| | hsgRNA-Q794*-3 | cugugcccag | 597 | gcaacuaugccugugcccag | 598 |

Table 11.33 sgRNA-G796K/R/E and
its hsgRNAs targeting G796 of BCL11A

| | | | | | |
|---|---|---|---|---|---|
| sgRNA | sgRNA-G796K/R/E | ccuggccaug | 417 | uccuuccccaccuggccaug | 418 |
| hsgRNA | hsgRNA-G796K/R/E-1 | cacgcuaaaa | 599 | ggguacuguacacgcuaaaa | 600 |
| | hsgRNA-G796K/R/E-2 | acacgcuaaa | 601 | aggguacuguacacgcuaaa | 602 |

Table 11.34 sgRNA-G796K/R/E-D798N and
its hsgRNAs targeting G796 and D798 of BCL11A

| | | | | | |
|---|---|---|---|---|---|
| sgRNA | sgRNA-G796K/R/E-D798N | caccuggcca | 419 | cguccuuccccaccuggcca | 420 |
| hsgRNA | hsgRNA-G796K/R/E-D798N-1 | cacgcuaaaa | 603 | ggguacuguacacgcuaaaa | 604 |
| | hsgRNA-G796K/R/E-D798N-2 | acacgcuaaa | 605 | aggguacuguacacgcuaaa | 606 |

Table 11.35 sgRNA-P808F/S/L and its
hsgRNAs targeting P808 of BCL11A

| | | | | | |
|---|---|---|---|---|---|
| sgRNA | sgRNA-P808F/S/L | uagcguguac | 421 | agaugccuuuuagcguguac | 422 |
| hsgRNA | hsgRNA-P808F/S/L-1 | uggggaagga | 607 | cauggccagguggggaagga | 608 |
| | hsgRNA-P808F/S/L-2 | agguggggaa | 609 | acgcauggccagguggggaa | 610 |
| | hsgRNA-P808F/S/L-3 | ggccaggugg | 611 | gaaaacgcauggccaggugg | 612 |

Table 11.36 sgRNA-S813N-1 and its
hsgRNAs targeting S813 of BCL11A

| | | | | | |
|---|---|---|---|---|---|
| sgRNA | sgRNA-S813N-1 | cacgcuaaaa | 423 | ggguacuguacacgcuaaaa | 424 |
| hsgRNA | hsgRNA-S813N-1-1 | ucgaucacug | 613 | uauucaacacucgaucacug | 614 |
| hsgRNA | hsgRNA-S813N-1-2 | acucgaucac | 615 | auuauucaacacucgaucac | 616 |

Table 11.37 sgRNA-S813N-2 and its
hsgRNAs targeting S813 of BCL11A

| | | | | | |
|---|---|---|---|---|---|
| sgRNA | sgRNA-S813N-2 | acacgcuaaa | 425 | aggguacuguacacgcuaaa | 426 |
| hsgRNA | hsgRNA-S813N-2-1 | ucgaucacug | 617 | uauucaacacucgaucacug | 618 |
| hsgRNA | hsgRNA-S813N-2-2 | acucgaucac | 619 | auuauucaacacucgaucac | 620 |

Table 11.38 sgRNA-E816K and its
hsgRNAs targeting E816 of BCL11A

| | | | | | |
|---|---|---|---|---|---|
| sgRNA | sgRNA-E816K | guacuguaca | 427 | uuucuccaggguacuguaca | 428 |
| hsgRNA | hsgRNA-E816K-1 | auucaacacu | 621 | uuauaucauuauucaacacu | 622 |

Table 11.39 sgRNA-R826* and its
hsgRNAs targeting R826 of BCLIIA

| | | | | | |
|---|---|---|---|---|---|
| sgRNA | sgRNA-R826* | uguugaauaa | 429 | agugaucgaguguugaauaa | 430 |
| hsgRNA | hsgRNA-R826*-1 | aguacccugg | 623 | uagcguguacaguacccugg | 624 |
| hsgRNA | hsgRNA-R826*-2 | acaguacccu | 625 | uuuagcguguacaguacccu | 626 |
| hsgRNA | hsgRNA-R826*-3 | uacaguaccc | 627 | uuuuagcguguacaguaccc | 628 |

### Example 3. Identification of a New Deaminase Inhibitor

A tBE includes, along with a base editor, a cytidine deaminase inhibitor to inhibit the activity of the cytidine deaminase. The inhibitor can be cleaved once the tBE complex is assembled at the target genomic site. This example tested a newly identified cytidine deaminase inhibitor, hA3F-CDA1.

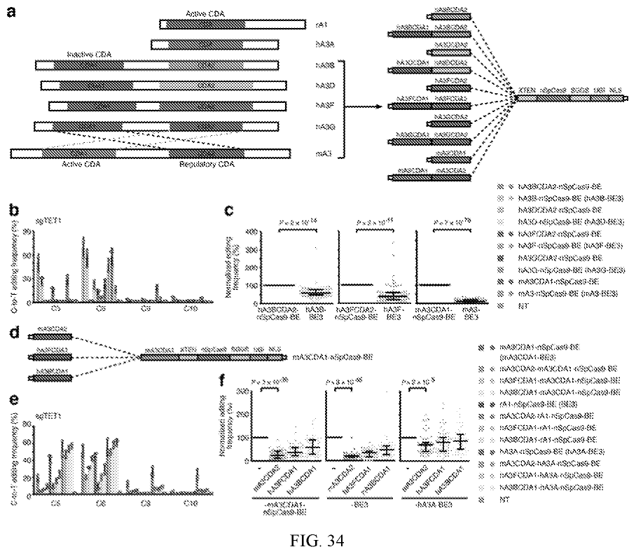

As illustrated in FIG. 34a, each of hA3B, hA3D, hA3F and hA3G contains a CDA1 domain, which was contemplated to correspond to the mouse mA3-CDA2 domain, which we have confirmed as a cytidine deaminase inhibitor. Base editing constructs with or without these potential inhibitors were prepared (panel on the right).

The editing frequencies of these base editors were measured at a representative genomic locus, and the results are charted in FIG. 34b. Statistical analysis of normalized editing frequencies was carried out (FIG. 34c), setting the ones induced by the single-CDA-containing BEs as 100%. n=78 (hA3BCDA2-nSpCas9-BE, hA3B-BE3, mA3CDA1-nSpCas9-BE and mA3-BE3) or 74 (hA3FCDA2-nSpCas9-BE and hA3F-BE3) edited cytosines at seven on-target sites from three independent experiments. The positive control mA3-BE3 exhibited the highest inhibitory effect and hA3F-CDA1 was the best among the tested candidate inhibitors.

Each of mA3-CDA2, hA3F-CDA1 and hA3B-CDA1 was fused to mA3-CDA1 (mA3CDA1-nSpCas9-BE) to prepare three tBE (FIG. 34d). As shown in the editing frequencies (FIG. 34e) and statistical analysis results (FIG. 34e), hA3F-CDA1 again outperformed hA3B-CDA1 in terms of the inhibitory activities.

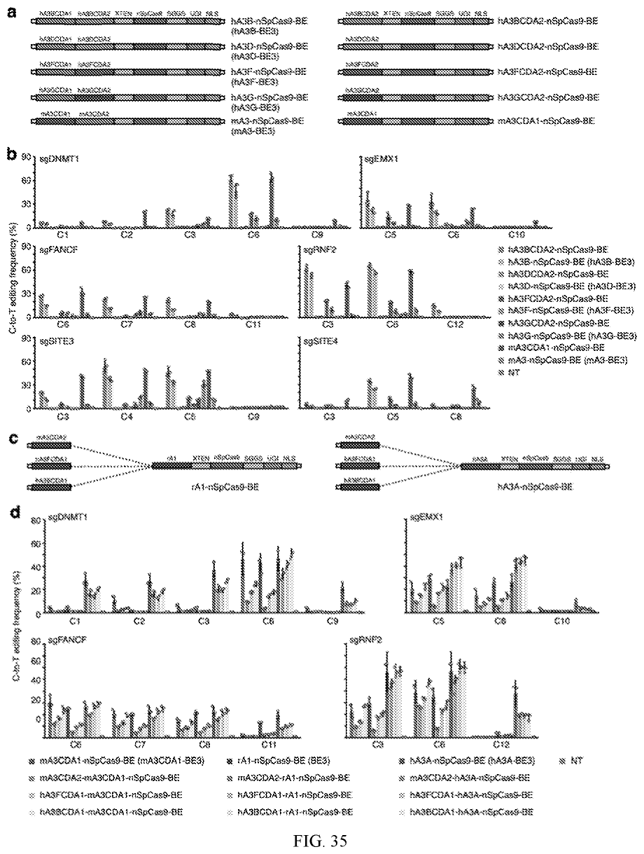

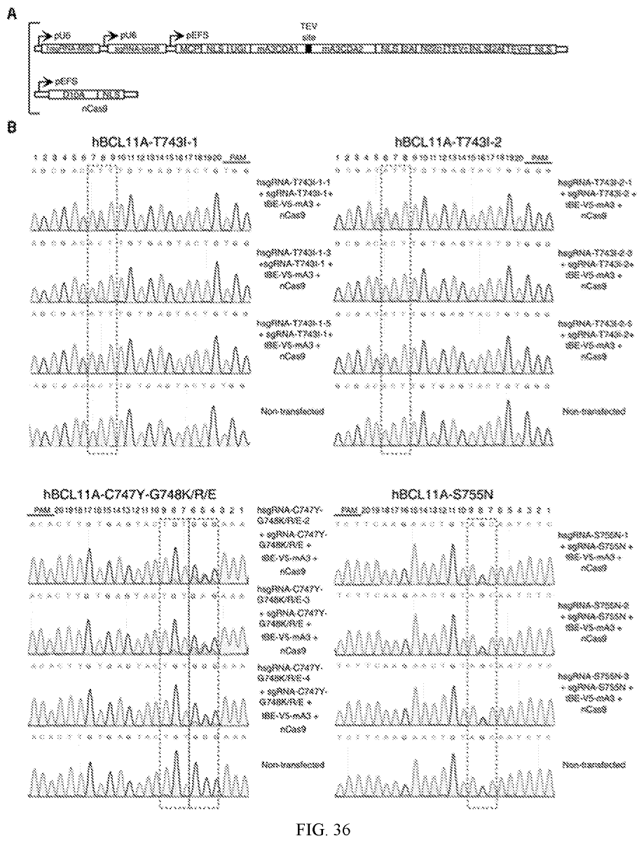

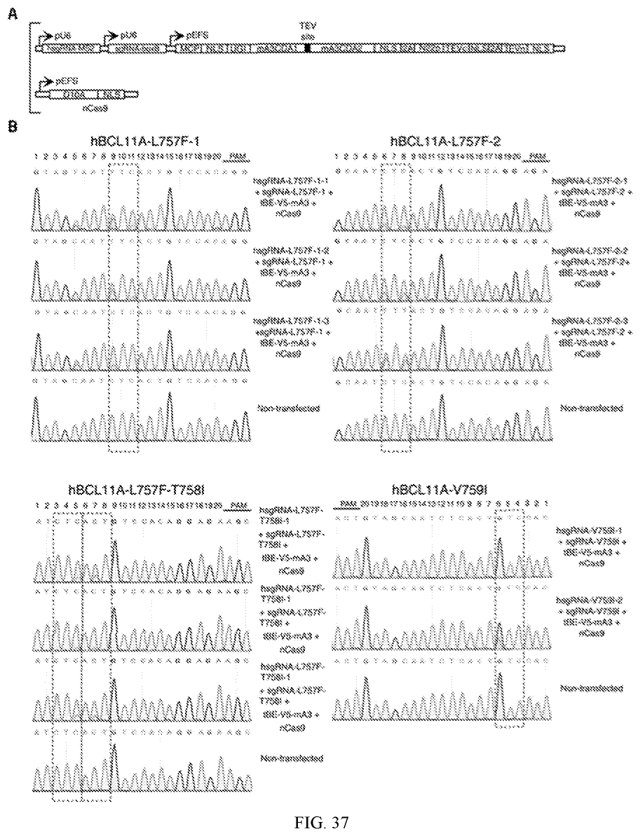

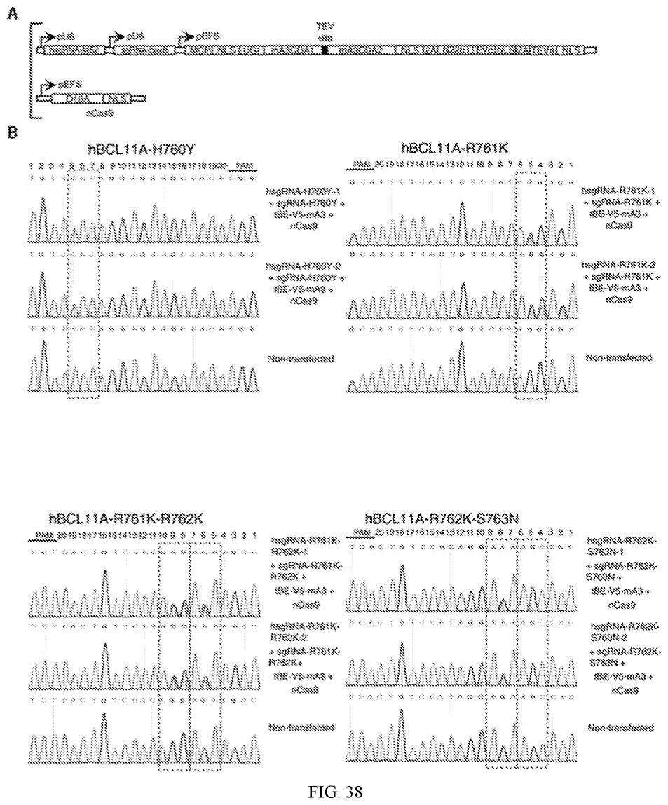

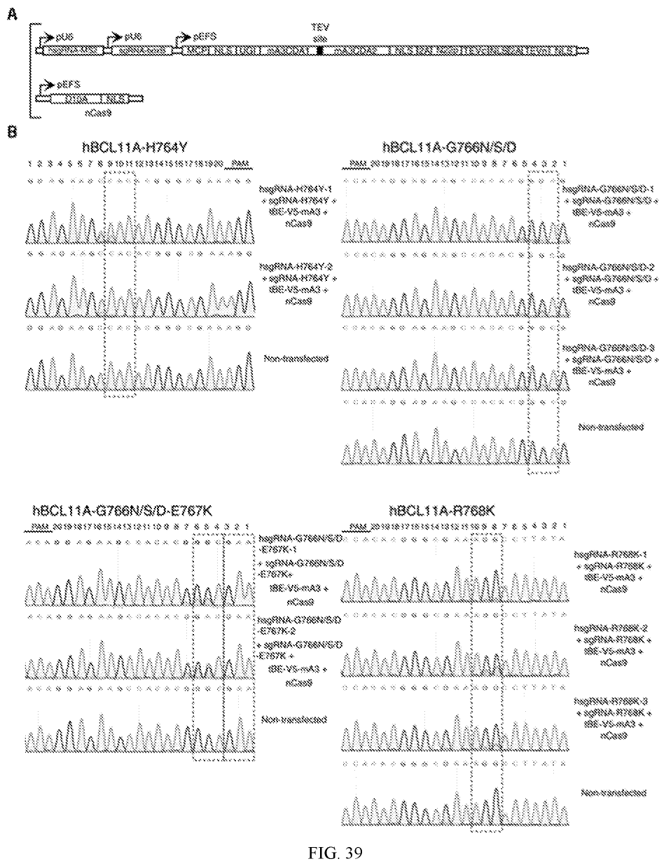

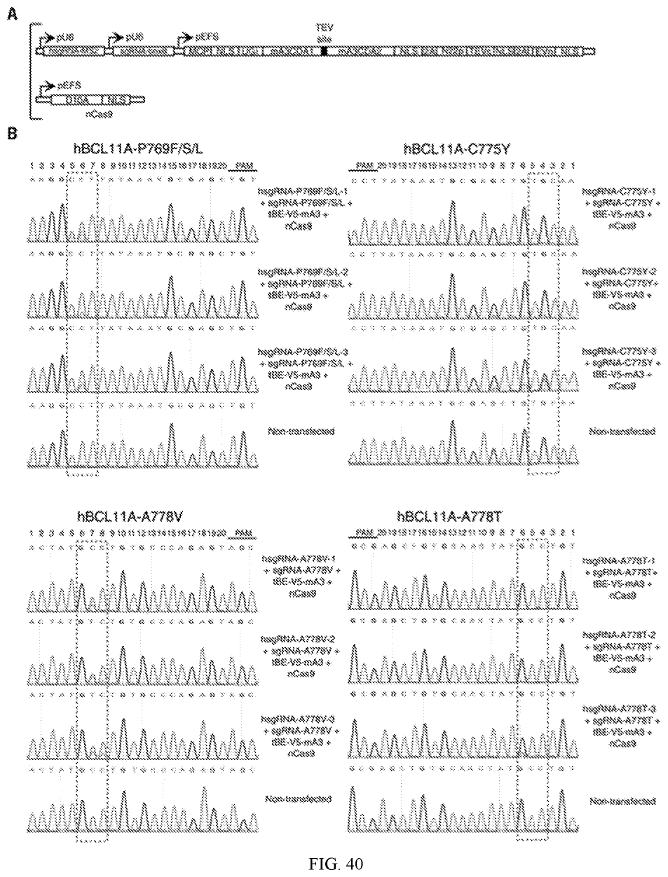

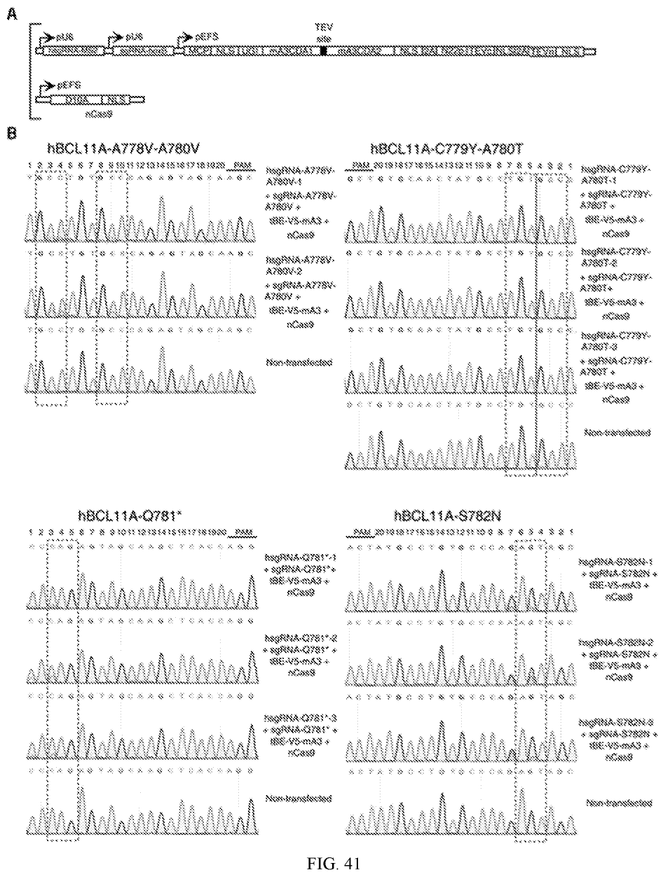

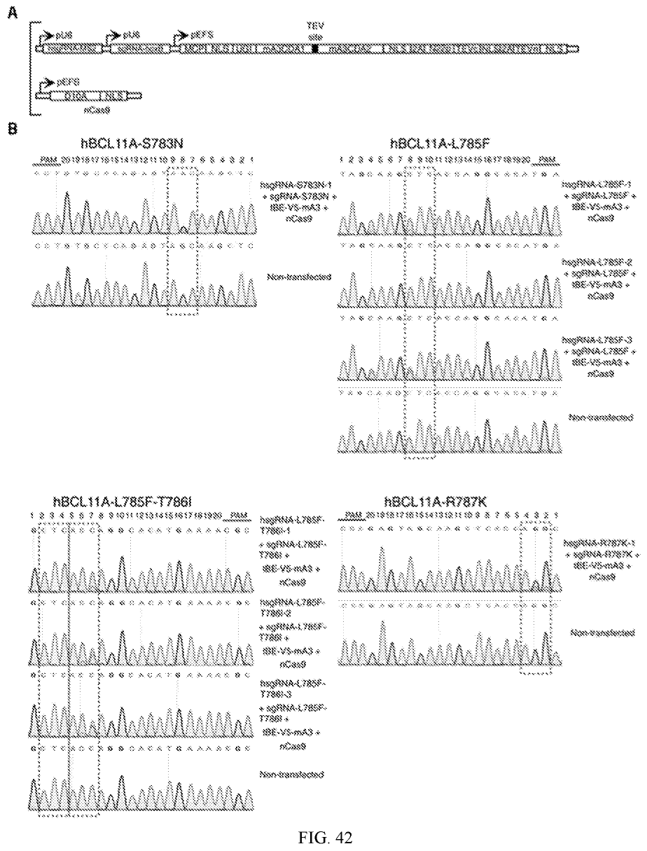

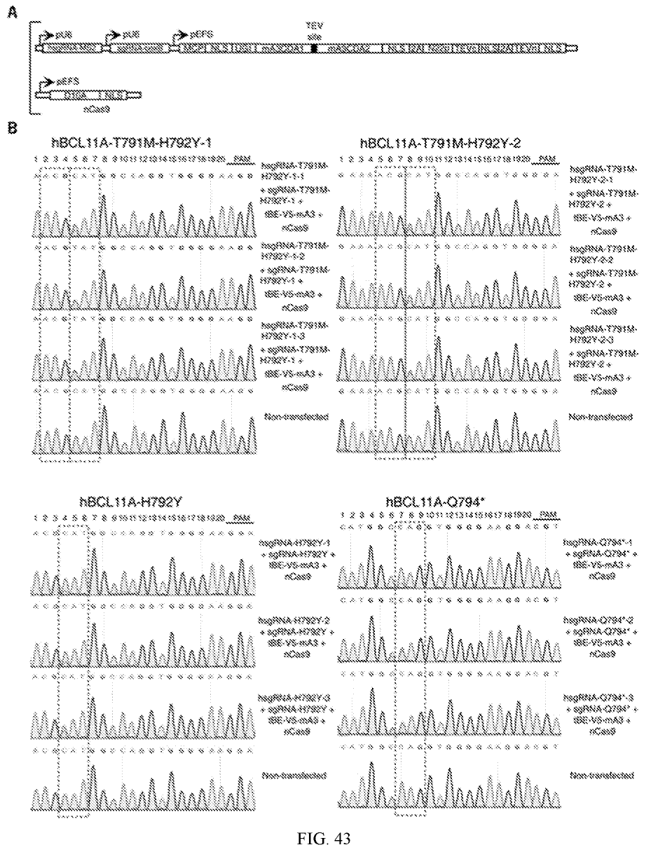

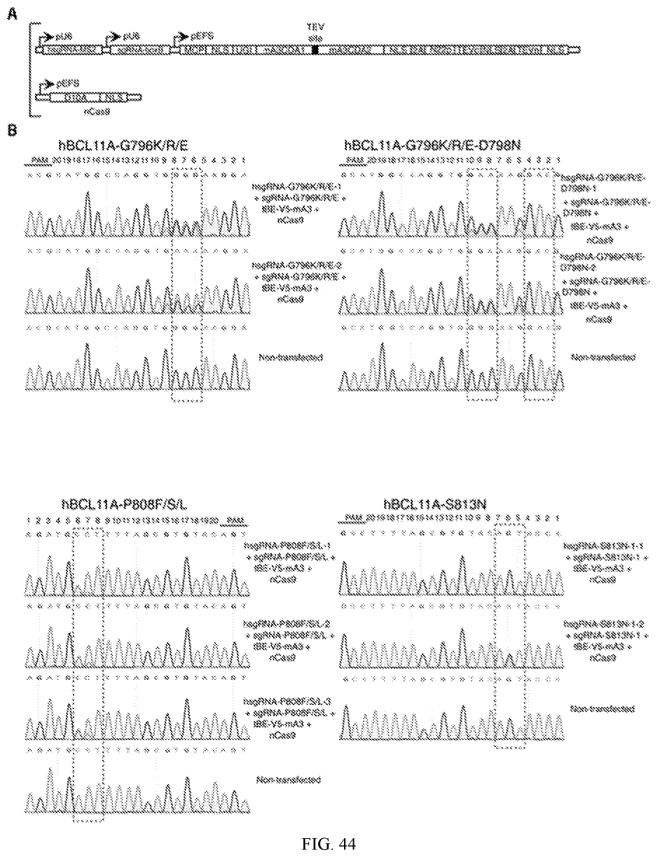

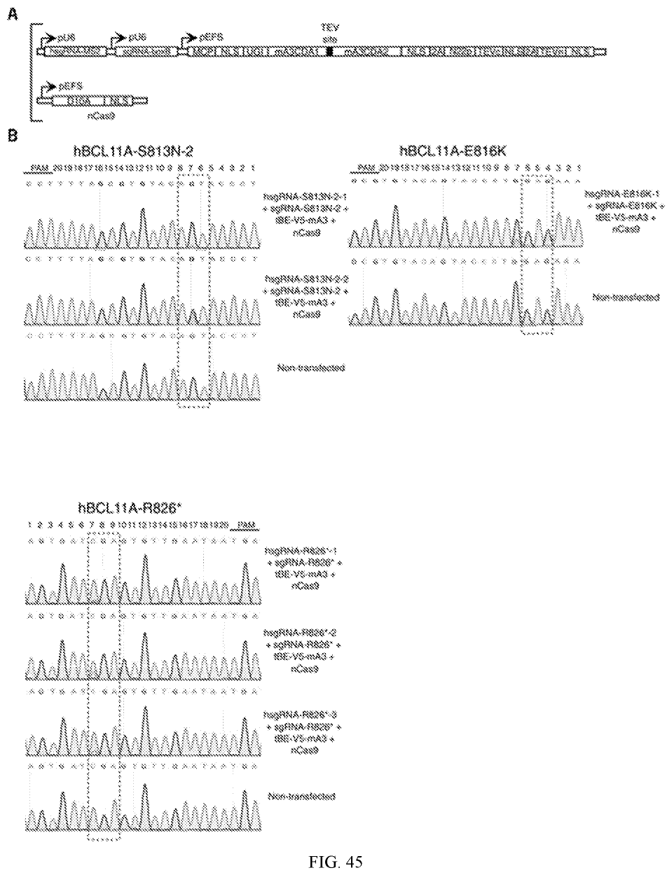

Similar constructs were prepared and tested for their inhibitory effects on C-to-T editing frequencies at six genomic loci (FIG. 35a) or C-to-T editing frequencies at four genomic loci (FIG. 35c). Again, hA3F-CDA1 exhibited the best inhibitory effects.

This example, therefore, identifies hA3F-CDA1 as an excellent cytidine deaminase inhibitor, suitable for preparing transformer Base Editors (tBE).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. A method for promoting production of 7-globin in a human cell, comprising introducing into the cell a CRISPR-associated (Cas) protein, a nucleobase deaminase, a single-guide RNA (sgRNA), and a helper single-guide RNA (hsgRNA), wherein
(a) the sgRNA comprises a nucleic acid sequence selected from the group consisting of SEQ ID NO:1-10, and the hsgRNA comprises a nucleic acid sequence selected from the group consisting of SEQ ID NO:11-28;
(b) the sgRNA comprises the nucleic acid sequence of SEQ ID NO:29-30, and the hsgRNA comprises a nucleic acid sequence selected from the group consisting of SEQ ID NO:31-36,
(c) the sgRNA comprises a nucleic acid sequence selected from the group consisting of SEQ ID NO:37-54, and the hsgRNA comprises a nucleic acid sequence selected from the group consisting of SEQ ID NO:63-116,
(d) the sgRNA comprises a nucleic acid sequence selected from the group consisting of SEQ ID NO:117-122, and the hsgRNA comprises a nucleic acid sequence selected from the group consisting of SEQ ID NO:123-138,
(e) the sgRNA comprises a nucleic acid sequence selected from the group consisting of SEQ ID NO:139-150, and the hsgRNA comprises a nucleic acid sequence selected from the group consisting of SEQ ID NO:151-190, or
(f) the sgRNA comprises a nucleic acid sequence selected from the group consisting of SEQ ID NO:353-430, and the hsgRNA comprises a nucleic acid sequence selected from the group consisting of SEQ ID NO:431-628, and
wherein the Cas protein, the nucleobase deaminase, the sgRNA, and the hsgRNA are preferably introduced into the cell by one or more encoding polynucleotides.

2. The method of claim 1, wherein the sgRNA comprises the nucleic acid sequence of SEQ ID NO:4, and the hsgRNA comprises the nucleic acid of SEQ ID NO:11.

3. The method of claim 1, wherein the sgRNA comprises the nucleic acid sequence of SEQ ID NO:30, and the hsgRNA comprises the nucleic acid of SEQ ID NO:33.

4. The method of claim 1, wherein the sgRNA and the hsgRNA comprise (b) and at least one pair selected from (a) and (c)-(e).

5. The method of claim 1, wherein the sgRNA and the hsgRNA comprise (a) and (b).

6. The method of claim 1, wherein:
the sgRNA comprises the nucleic acid sequence of SEQ ID NO:38, and the hsgRNA comprises a nucleic acid sequence selected from the group consisting of SEQ ID NO:55-62, preferably SEQ ID NO:57 or 59;
the sgRNA comprises the nucleic acid sequence of SEQ ID NO:40, and the hsgRNA comprises a nucleic acid sequence selected from the group consisting of SEQ ID NO:63-70, preferably SEQ ID NO:57 or 59;
the sgRNA comprises the nucleic acid sequence of SEQ ID NO:42, and the hsgRNA comprises a nucleic acid sequence selected from the group consisting of SEQ ID NO:71-80, preferably SEQ ID NO:71, 73, 77 or 79;
the sgRNA comprises the nucleic acid sequence of SEQ ID NO:44, and the hsgRNA comprises a nucleic acid sequence selected from the group consisting of SEQ ID NO:81-104, preferably SEQ ID NO:81, 85 or 101;
the sgRNA comprises the nucleic acid sequence of SEQ ID NO:46, and the hsgRNA comprises a nucleic acid sequence selected from the group consisting of SEQ ID NO:81-86 and SEQ ID NO:99-104, preferably SEQ ID NO:81, 85 or 101;
the sgRNA comprises the nucleic acid sequence of SEQ ID NO:48, and the hsgRNA comprises a nucleic acid sequence selected from the group consisting of SEQ ID NO: 81-86 and SEQ ID NO:99-104, preferably SEQ ID NO:85;
the sgRNA comprises the nucleic acid sequence of SEQ ID NO:50, and the hsgRNA comprises a nucleic acid sequence selected from the group consisting of SEQ ID NO:87-98, preferably SEQ ID NO:87, 89, 91 or 93;
the sgRNA comprises the nucleic acid sequence of SEQ ID NO:52, and the hsgRNA comprises a nucleic acid sequence selected from the group consisting of SEQ ID NO:105-116, preferably SEQ ID NO:111, 113 or 115; or
the sgRNA comprises the nucleic acid sequence of SEQ ID NO:54, and the hsgRNA comprises a nucleic acid sequence selected from the group consisting of SEQ ID NO:105-115.

7. The method of claim 1, wherein:
the sgRNA comprises the nucleic acid sequence of SEQ ID NO:118, and the hsgRNA comprises a nucleic acid sequence selected from the group consisting of SEQ ID NO:123-138, preferably SEQ ID NO:127 or 129;
the sgRNA comprises the nucleic acid sequence of SEQ ID NO:120, and the hsgRNA comprises a nucleic acid sequence selected from the group consisting of SEQ ID NO:123-138, preferably SEQ ID NO:123, 127 or 129; or
the sgRNA comprises the nucleic acid sequence of SEQ ID NO:122, and the hsgRNA comprises a nucleic acid sequence selected from the group consisting of SEQ ID NO:123-138.

8. The method of claim 1, wherein:
the sgRNA comprises the nucleic acid sequence of SEQ ID NO:140, and the hsgRNA comprises a nucleic acid sequence selected from the group consisting of SEQ ID NO:151-164, preferably SEQ ID NO:153, 155, 157, 159 or 163;
the sgRNA comprises the nucleic acid sequence of SEQ ID NO:142, and the hsgRNA comprises a nucleic acid sequence selected from the group consisting of SEQ ID NO:151-164, preferably SEQ ID NO:159, 161 or 163;
the sgRNA comprises the nucleic acid sequence of SEQ ID NO:144, and the hsgRNA comprises a nucleic acid sequence selected from the group consisting of SEQ ID NO:151-164, preferably SEQ ID NO:151, 161 or 163;
the sgRNA comprises the nucleic acid sequence of SEQ ID NO:146, and the hsgRNA comprises a nucleic acid sequence selected from the group consisting of SEQ ID NO:151-164;
the sgRNA comprises the nucleic acid sequence of SEQ ID NO:148, and the hsgRNA comprises a nucleic acid sequence selected from the group consisting of SEQ ID NO:165-178, preferably SEQ ID NO:165, 169, 171, 173, 175 or 177; or
the sgRNA comprises the nucleic acid sequence of SEQ ID NO:150, and the hsgRNA comprises a nucleic acid sequence selected from the group consisting of SEQ ID NO:179-190, preferably SEQ ID NO:185, 187 or 189.

9. The method of claim 1, wherein the nucleobase deaminase is a cytidine deaminase.

10. The method of claim 9, wherein the cytidine deaminase is selected from the group consisting of APOBEC3B (A3B), APOBEC3C (A3C), APOBEC3D (A3D), APOBEC3F (A3F), APOBEC3G (A3G), APOBEC3H (A3H), APOBEC1 (A1), APOBEC3 (A3), APOBEC2 (A2), APOBEC4 (A4) and AICDA (AID).

11. The method of claim 1, further comprising introducing into the cell a nucleobase deaminase inhibitor, fused to the nucleobase deaminase, via a protease cleavage site.

12. The method of claim 11, wherein the nucleobase deaminase inhibitor is an inhibitory domain of a nucleobase deaminase.

13. The method of claim 11, wherein the nucleobase deaminase inhibitor is an inhibitory domain of a cytidine deaminase.

14. The method of claim 11, wherein the nucleobase deaminase inhibitor comprises an amino acid sequence selected from SEQ ID NO:191-193.

15. The method of claim 1, further comprising introducing into the cell a protease that is capable of cleaving at the protease cleavage site.

16. The method of claim 15, wherein the protease is selected from the group consisting of TuMV protease, PPV protease, PVY protease, ZIKV protease and WNV protease.

17. The method of claim 1, wherein the Cas protein is selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

18-21. (canceled)

22. The method of claim 1, wherein the patient suffers from (3-thalassemia, sickle cell anemia, Haemoglobin C, or Haemoglobin E.

23. One or more polynucleotides encoding a CRISPR-associated (Cas) protein, a nucleobase deaminase, a single-guide RNA (sgRNA), and a helper single-guide RNA (hsgRNA), wherein
(a) the sgRNA comprises a nucleic acid sequence selected from the group consisting of SEQ ID NO:1-10, and the hsgRNA comprises a nucleic acid sequence selected from the group consisting of SEQ ID NO:11-28;
(b) the sgRNA comprises the nucleic acid sequence of SEQ ID NO:29-30, and the hsgRNA comprises a nucleic acid sequence selected from the group consisting of SEQ ID NO:31-36,
(c) the sgRNA comprises a nucleic acid sequence selected from the group consisting of SEQ ID NO:37-54, and the hsgRNA comprises a nucleic acid sequence selected from the group consisting of SEQ ID NO:63-116,
(d) the sgRNA comprises a nucleic acid sequence selected from the group consisting of SEQ ID NO:118-122, and the hsgRNA comprises a nucleic acid sequence selected from the group consisting of SEQ ID NO:123-138, or
(e) the sgRNA comprises a nucleic acid sequence selected from the group consisting of SEQ ID NO:139-150, and the hsgRNA comprises a nucleic acid sequence selected from the group consisting of SEQ ID NO:151-190.

24-26. (canceled)

27. A fusion protein comprising:
a first fragment comprising a cytidine deaminase or a catalytic domain thereof,
a second fragment comprising a cytidine deaminase inhibitor comprising an amino acid sequence selected from the group consisting of SEQ ID NO:192, and 265-309 and sequences having at least 85% sequence identity to any of SEQ ID NO:192, and 265-309, and
a protease cleavage site between the first fragment and the second fragment.

28-32. (canceled)
